(19) United States
(12) Patent Application Publication
(10) Pub. No.: US 2013/0224234 A1
(43) Pub. Date: Aug. 29, 2013

(54) TTLL4 PEPTIDES AND VACCINES CONTAINING THE SAME

(71) Applicants: Yusuke Nakamura, Tokyo (JP); Takuya Tsunoda, Kanagawa (JP); Ryuji Osawa, Kanagawa (JP)

(72) Inventors: Yusuke Nakamura, Tokyo (JP); Takuya Tsunoda, Kanagawa (JP); Ryuji Osawa, Kanagawa (JP)

(73) Assignee: Onco Therapy Science, Inc., Kawasaki-shi, Kanagawa (JP)

(21) Appl. No.: 13/820,736

(22) PCT Filed: Sep. 6, 2011
(86) PCT No.: PCT/JP2011/004987

§ 371 Date: May 17, 2013

Related U.S. Application Data
(60) Provisional application No. 61/380,611, filed on Sep. 7, 2010.

(51) Int. Cl. C12N 9/00; C07K 16/40

(52) U.S. Cl. CPC C12N 9/93; C07K 16/40
USPC 424/185.1; 530/328; 536/23.5; 435/455; 435/325; 424/277.1; 530/387.9; 435/320.1; 435/365

(57) ABSTRACT

Peptide vaccines against cancer are described herein. In particular, epitope peptides derived from the TTLL4 gene that elicit CTLs are provided. Antigen-presenting cells and isolated CTLs that target such peptides, as well as methods for inducing the antigen-presenting cell, or CTL are also provided. The present invention further provides pharmaceutical compositions containing peptides derived from TTLL4 or polynucleotides encoding the polypeptides as active ingredients. Furthermore, the present invention provides methods for the treatment and/or prophylaxis of (i.e., preventing) cancers (tumors), and/or the prevention of a postoperative recurrence thereof, as well as methods for inducing CTLs, methods for inducing anti-tumor immunity, using the peptides derived from TTLL4, polynucleotides encoding the peptides, or antigen-presenting cells presenting the peptides, or the pharmaceutical compositions of the present invention.

### PRIORITY

The present application claims the benefit of U.S. Provisional Application No. 61/380,611, filed on Sep. 7, 2010, the entire contents of which are incorporated by reference herein.

### TECHNICAL FIELD

The present invention relates to the field of biological science, more specifically to the field of cancer therapy. In particular, the present invention relates to novel peptides that are effective as cancer vaccines, drugs for treating and preventing tumors, as well as methods for diagnosing tumors.

### BACKGROUND ART

It has been demonstrated that CD8 positive cytotoxic T lymphocytes (CTLs) recognize epitope peptides derived from the tumor-associated antigens (TAAs) found on the major histocompatibility complex (MHC) class I molecule, and then kill the tumor cells. Since the discovery of the melanoma antigen (MAGE) family as the first example of TAAs, many other TAAs have been discovered, primarily through immunological approaches (NPL 1; NPL 2). Some of these TAAs are in currently undergoing clinical development as immunotherapeutic targets.

Favorable TAAs are indispensable for the proliferation and survival of cancer cells. The use of such TAAs as targets for immunotherapy may minimize the well-described risk of immune escape of cancer cells attributable to deletion, mutation, or down-regulation of TAAs as a consequence of therapeutically driven immune selection. Accordingly, the identification of new TAAs capable of inducing potent and specific anti-tumor immune responses warrants further development and thus clinical application of peptide vaccination strategies for various types of cancer in ongoing (NPL 3; NPL 4; NPL 5; NPL 6; NPL 7; NPL 8; NPL 9; NPL 10). To date, there have been several reports of clinical trials using these TAA derived peptides. Unfortunately, only a low objective response rate has been observed in these cancer vaccine trials (NPL 11; NPL 12; NPL 13). Accordingly, there remains a need for new TAAs as immunotherapeutic targets.

TTLL4 (GenBank Accession No: NP_{—}055455), tublin tyrosine ligase-like family member 4, is a polyglutamylase enzyme. It plays an important role in several microtubule functions. Polyglutamylation is a reversible modification generated by sequential covalent attachment of glutamic acids to an internal glutamate residue of the target protein (NPL 14). Its biological significance is not well known. The only known targets of polyglutamylation are alpha- and beta-tublins, the structural units of microtubules (NPL 15), and the nucleosome assembly proteins, NAP1 and NAP2 (NPL 16).

Genome-wide gene expression profile analysis of pancreatic ductal adenocarcinoma (PDAC) cells indicated that TTLL4 is over-expressed in PDAC. Furthermore, knockdown of TTLL4 by siRNA in PDAC cells attenuated the growth of PDAC cells and exogenous introduction of TTLL4 enhanced the cell growth (NPL 17). Northern blotting analysis demonstrate that TTLL4 is not expressed in normal organs expect for testis.

Taken together, these data suggests that TTLL4 may be a suitable target for cancer immunotherapy protocols, particularly for patient with TTLL4 expressing tumors.

### CITATION LIST

### Non Patent Literature

[NPL 1] Boon T, Int J Cancer 1993, 54(2): 177-80
[NPL 2] Boon T & van der Bruggen P, J Exp Med 1996, 183(3): 725-9
[NPL 3] Harris C C, J Natl Cancer Inst 1996, 88(20): 1442-55
[NPL 4] Butterfield L H et al., Cancer Res 1999, 59(13): 3134-42
[NPL 5] Vissers J L et al., Cancer Res 1999, 59(21): 5554-9
[NPL 6] van der Burg S H et al., J Immunol 1996, 156(9): 3308-14
[NPL 7] Tanaka F et al., Cancer Res 1997, 57(20): 4465-8
[NPL 8] Fujie T et al., Int J Cancer 1999, 80(2): 169-72
[NPL 9] Kikuchi M et al., Int J Cancer 1999, 81(3): 459-66
[NPL 10] Oiso M et al., Int J Cancer 1999, 81(3): 387-94
[NPL 11] Belli F et al., J Clin Oncol 2002, 20(20): 4169-80
[NPL 12] Coulie P G et al., Immunol Rev 2002, 188: 33-42
[NPL 13] Rosenberg S A et al., Nat Med 2004, 10(9): 909-15
[NPL 14] Edde B. et al., Science. 1990; 247(4938): 83-5
[NPL 15] Rudiger M. et al., FEBS Lett. 1992; 308(1): 101-5
[NPL 16] Regnard C., J Biol. Chem. 2000; 275(21): 15969-76
[NPL 17] Kotoe K., Cancer Res. 2010; 70(10): 4024-33

### SUMMARY OF INVENTION

The present invention is based, at least in part, on the discovery of novel peptides that may serve as suitable targets of immunotherapy. Because TAAs are generally perceived by the immune system as “self” and therefore often have no innate immunogenicity, the discovery of appropriate targets is of extreme importance. Through the present invention, TTLL4 (SEQ ID NO: 80 encoded by the gene of GenBank Accession No. NM_{—}014640 (SEQ ID NO: 79)) is demonstrated to be specifically over-expressed in cancer cells, in particular bladder cancer, cholangiocellular carcinoma, chronic myelogenous leukemia (CML), colon and rectum cancer, esophageal cancer, liver cancer, lymphoma, pancreatic cancer, prostate cancer, renal carcinoma, small-cell lung cancer (SCLC), non-small-cell lung cancer (NSCLC), soft tissue tumor and osteosarcoma, but not limited thereto. Thus, the present invention focuses on TTLL4 as an appropriate cancer marker and candidate for the target of immunotherapy.

The present invention further relates to the identification of specific epitope peptides among the gene products of TTLL4 that possess the ability to induce CTLs specific to TTLL4. As discussed in detail below, peripheral blood mononuclear cells (PBMCs) obtained from a healthy donor were stimulated using HLA-A*2402 or HLA-A*0201 binding candidate peptides derived from TTLL4. CTL lines were then established with specific cytotoxicity against the HLA-A24 or HLA-A2 positive target cells pulsed with each of candidate peptides. The results herein demonstrate that these peptides are HLA-A24 or HLA-A2 restricted epitope peptides that can induce potent and specific immune responses against cells expressing TTLL4. These results further indicate that TTLL4 is strongly immunogenic and that the epitopes thereof are effective targets for tumor immunotherapy.

Accordingly, it is an object of the present invention is to provide isolated peptides that bind to HLA antigen and include the TTLL4 sequence (SEQ ID NO: 80) or an immunogenic fragment thereof. These peptides are expected to have CTL inducibility and, thus, can be used to induce CTL in vitro, ex vivo or in vivo or to be administered to a subject for inducing immune responses against cancers, examples of which include, but are not limited to, bladder cancer, cholangiocellular carcinoma, CML, colon and rectum cancer, esophageal cancer, liver cancer, lymphoma, pancreatic cancer, prostate cancer, renal carcinoma, SCLC, NSCLC, soft tissue tumor and osteosarcoma but not limited thereto. Preferred peptides are nonapeptides and decapeptides, and more preferably nonapeptides and decapeptides having an amino acid sequence selected from among SEQ ID NOs: 1, 3 to 37 and 38 to 73. Of these, the peptides having an amino sequence selected from among SEQ ID NOs: 1, 6, 11, 12, 16, 20, 21, 22, 28, 29, 32, 37, 38, 39, 44 and 59 are most preferred.

The present invention also contemplates modified peptides having an amino acid sequence selected from among SEQ ID NOs: 1, 3 to 37 and 38 to 73 in which one, two or more amino acids are substituted, deleted, inserted or added, so long as the resulting modified peptides retain the requisite CTL inducibility and HLA binding ability of the original unmodified peptide.

The present invention further encompasses isolated polynucleotides encoding any one of peptides of the present invention. These polynucleotides can be used to induce or prepare APCs having CTL inducibility. Like the above-described peptides of the present invention, such APCs can be administered to a subject for inducing immune responses against cancers.

When administered to a subject, the peptides of the present invention are preferably presented on the surface of APCs so as to induce CTLs targeting the respective peptides. Therefore, one object of the present invention is to provide agents and/or compositions that induce CTL, such compositions or agents including one or more peptides of the present invention, or polynucleotides encoding such peptides. Such agents, substances, and/or compositions can be used for the treatment and/or prophylaxis of a primary cancer, a metastasis or post-operative recurrence thereof. Examples of cancers contemplated by the present invention include, but are not limited to, bladder cancer, cholangiocellular carcinoma, CML, colon and rectum cancer, esophageal cancer, liver cancer, lymphoma, pancreatic cancer, prostate cancer, renal carcinoma, SCLC, NSCLC, soft tissue tumor and osteosarcoma.

The present invention further contemplates pharmaceutical compositions or agents that include or incorporate one or more peptides or polynucleotides of the present invention formulated for the treatment and/or prophylaxis of a primary cancer, metastasis or postoperative recurrence as noted above. Instead of or in addition to the present peptides or polynucleotides, the present pharmaceutical agents and/or compositions may include as active ingredients APCs or exosomes that present any of the present peptides.

The peptides or polynucleotides of the present invention may be used to induce APCs that present on the surface a complex of an HLA antigen and a peptide of the present invention, for example, by contacting APCs derived from a subject with the peptide or introducing a polynucleotide encoding a peptide of this invention into APCs. Such APCs have high CTL inducibility against target peptides and are useful for cancer immunotherapy. Accordingly, the present invention encompasses the methods for inducing APCs with CTL inducibility as well as the APCs obtained by the methods.

It is a further object of the present invention to provide methods for inducing CTL, such methods include the step of co-culturing CD8 positive T cells with APCs presenting on its surface a complex of an HLA antigen and one or more peptides of the present invention, the step of co-culturing CD8 positive T cells with exosomes presenting on its surface a complex of an HLA antigen and one or more peptides of the present invention, or the step of introducing a gene that includes one or more polynucleotides coding for a T cell receptor (TCR) subunit polypeptide that binds to a peptide of the present invention. CTLs obtained by such methods find use in the treatment and/or prevention of cancers, more particularly bladder cancer, cholangiocellular carcinoma, CML, colon and rectum cancer, esophageal cancer, liver cancer, lymphoma, pancreatic cancer, prostate cancer, renal carcinoma, SCLC, NSCLC, soft tissue tumor and osteosarcoma. Therefore, it is yet another object of the present invention to provide CTLs.

Yet another object of the present invention is to provide isolated APCs that present on the surface a complex of an HLA antigen and a peptide of the present invention. The present invention further provides isolated CTLs that target peptides of the present invention. These APCs and CTLs may be used for cancer immunotherapy.

It is yet another object of the present invention to provide methods for inducing an immune response against a cancer in a subject in need thereof, such methods including the step of administering to the subject a composition including a peptide of the present invention or a polynucleotide encoding such a peptide.

The applicability of the present invention extends to any of a number of diseases relating to or arising from TTLL4 overexpression, examples of which include, but are not limited to, bladder cancer, cholangiocellular carcinoma, CML, colon and rectum cancer, esophageal cancer, liver cancer, lymphoma, pancreatic cancer, prostate cancer, renal carcinoma, SCLC, NSCLC, soft tissue tumor and osteosarcoma. In addition to the above, other objects and features of the invention will become more fully apparent when the following detailed description is read in conjunction with the accompanying figures and examples. However, it is to be understood that both the foregoing summary of the invention and the following detailed description are of exemplified embodiments, and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### BRIEF DESCRIPTION OF DRAWINGS

Various aspects and applications of the present invention will become apparent to the skilled artisan upon consideration of the brief description of the figures and the detailed description of the present invention and its preferred embodiments that follows.

FIG. 1-1 is composed of a series of photographs, (a)-(j), depicting the results of IFN-gamma ELISPOT assay on CTLs that were induced with peptides derived from TTLL4. The CTLs in well number #7 with TTLL4-A24-9-750 (SEQ ID NO:1) (a), in #8 with TTLL4-A24-9-79 (SEQ ID NO:6) (b), in #8 with TTLL4-A24-9-793 (SEQ ID NO:11) (c), in #5 with TTLL4-A24-9-691 (SEQ ID NO:12) (d), in #1 with TTLL4-A24-9-103 (SEQ ID NO:16) (e), in #3 with TTLL4-A24-10-103 (SEQ ID NO:20) (f), in #3 with TTLL4-A24-10-773 (SEQ ID NO:21) (g), in #8 with TTLL4-A24-10-883 (SEQ ID NO:22) (h), in #2 with TTLL4-A24-10-1186 (SEQ ID NO:28) (i), in #3 with TTLL4-A24-10-1022 (SEQ ID NO:29) (j) showed potent IFN-gamma production compared with the control, respectively. The square on the well of these pictures indicates that the cells from the corresponding well were expanded to establish CTL lines. In the figures, “+” indicates the IFN-gamma production against target cells pulsed with the appropriate peptide, and “−” indicates the IFN-gamma production against target cells not pulsed with any peptides.

FIG. 1-2 is composed of a series of photographs, (k)-(m), depicting the results of IFN-gamma ELISPOT assay on CTLs that were induced with peptides derived from TTLL4. The CTLs in well number #1 with TTLL4-A24-10-994 (SEQ ID NO:32) (k) and in #6 with TTLL4-A24-10-891 (SEQ ID NO:37) (1) showed potent IFN-gamma production compared with the control, respectively. The square on the well of these pictures indicates that the cells from the corresponding well were expanded to establish CTL lines. In contrast, as is the typical case for negative data, no specific IFN-gamma production was detected from the CTLs stimulated with TTLL4-A24-9-579 (SEQ ID NO:2) (m). In the figures, “+” indicates the IFN-gamma production against target cells pulsed with the appropriate peptide, and “−” indicates the IFN-gamma production against target cells not pulsed with any peptides.

FIG. 2 is composed of a series of line graphs, (a)-(f), depicting the results of an IFN-gamma ELISA assay that, in turn demonstrates the IFN-gamma production of the CTL lines stimulated with TTLL4-A24-9-750 (SEQ ID NO:1) (a), TTLL4-A24-9-79 (SEQ ID NO:6) (b), TTLL4-A24-9-691 (SEQ ID NO:12) (c), TTLL4-A24-9-103 (SEQ ID NO:16) (d), TTLL4-A24-10-103 (SEQ ID NO:20) (e) and TTLL4-A24-10-773 (SEQ ID NO:21) (f). The results demonstrate that CTL lines established by stimulation with each peptide show potent IFN-gamma production as compared with the control. In the figures, “+” indicates the IFN-gamma production against target cells pulsed with the appropriate peptide, and “−” indicates the IFN-gamma production against target cells not pulsed with any peptides.

FIG. 3 is composed of a series of line graphs, (a)-(d), depicting the IFN-gamma production of the CTL clones established by limiting dilution from the CTL lines stimulated with TTLL4-A24-9-750 (SEQ ID NO:1) (a), TTLL4-A24-9-79 (SEQ ID NO:6) (b), TTLL4-A24-10-103 (SEQ ID NO:20) (c) and TTLL4-A24-10-773 (SEQ ID NO:21) (d). The results demonstrate that the CTL clones established by stimulation with each peptide show potent IFN-gamma production as compared with the control. In the figure, “+” indicates the IFN-gamma production against target cells pulsed with the appropriate peptide and “−” indicates the IFN-gamma production against target cells not pulsed with any peptides.

FIG. 4 is composed of a series of line graphs, (a)-(c), depicting specific CTL activity against target cells that exogenously express TTLL4 and HLA-A*2402. COS7 cells transfected with HLA-A*2402 or the full length TTLL4 gene were prepared as the controls. The CTL line established with TTLL4-A24-9-103 (SEQ ID NO: 16) (a) and the CTL clones established with TTLL4-A24-10-103 (SEQ ID NO: 20) (b) and TTLL4-A24-10-773 (SEQ ID NO: 21) (c) showed specific CTL activity against COS7 cells transfected with both TTLL4 and HLA-A*2402 (black lozenge). On the other hand, no significant specific CTL activity was detected against target cells expressing either HLA-A*2402 (triangle) or TTLL4 (circle).

FIG. 5 is composed of a series of photographs, (a)-(d), depicting the results of IFN-gamma ELISPOT assay on CTLs that were induced with peptides derived from TTLL4. The CTLs in well number #3 with TTLL4-A02-9-222 (SEQ ID NO:38) (a), in #7 with TTLL4-A02-9-805 (SEQ ID NO:39) (b), in #8 with TTLL4-A02-9-66 (SEQ ID NO:44) (c) and in #7 with TTLL4-A02-10-574 (SEQ ID NO:59) (d) showed potent IFN-gamma production as compared with the control, respectively. The square on the well of these pictures indicates that the cells from the corresponding well were expanded to establish CTL lines. In the figures, “+” indicates the IFN-gamma production against target cells pulsed with the appropriate peptide, and “−” indicates the IFN-gamma production against target cells not pulsed with any peptides.

FIG. 6 is composed of a series of line graphs, (a)-(d), depicting the results of an IFN-gamma ELISA assay that, in turn demonstrates IFN-gamma production of the CTL lines stimulated with TTLL4-A02-9-222 (SEQ ID NO:38) (a), TTLL4-A02-9-805 (SEQ ID NO:39) (b), TTLL4-A02-9-66 (SEQ ID NO:44) (c) and TTLL4-A02-10-574 (SEQ ID NO:59) (d) detected by IFN-gamma ELISA assay. The results demonstrate that CTL lines established by stimulation with each peptide show potent IFN-gamma production as compared with the control. In the figures, “+” indicates the IFN-gamma production against target cells pulsed with the appropriate peptide, and “−” indicates the IFN-gamma production against target cells not pulsed with any peptides.

FIG. 7 is composed of a series of line graphs, (a)-(c), depicting the results of an IFN-gamma ELISA assay that, in turn demonstrates the IFN-gamma production of the CTL clones established by limiting dilution from the CTL lines stimulated with TTLL4-A02-9-222 (SEQ ID NO:38) (a), TTLL4-A02-9-805 (SEQ ID NO:39) (b) and TTLL4-A02-10-574 (SEQ ID NO:59) (c). The results demonstrate that the CTL clones established by stimulation with each peptide show potent IFN-gamma production as compared with the control. In the figure, “+” indicates the IFN-gamma production against target cells pulsed with the appropriate peptide and “−” indicates the IFN-gamma production against target cells not pulsed with any peptides.

FIG. 8 is composed of a series of line graphs depicting specific CTL activity against target cells that exogenously express TTLL4 and HLA-A*0201. COS7 cells transfected with HLA-A*0201 or the full length TTLL4 gene were prepared as the controls. The CTL clone established with TTLL4-A02-9-805 (SEQ ID NO: 39) (a) and the CTL line established with TTLL4-A02-9-66 (SEQ ID NO: 44) (b) showed specific CTL activity against COS7 cells transfected with both TTLL4 and HLA-A*0201 (black lozenge). On the other hand, no significant specific CTL activity was detected against target cells expressing either HLA-A*0201 (triangle) or TTLL4 (circle).

### DESCRIPTION OF EMBODIMENTS

Further to the summary above, it is an object of the present invention to provide:

[1] An isolated peptide having CTL inducibility, wherein the peptide consists of the amino acid sequence of TTLL4 or an immunologically active fragment thereof.

[2] The isolated peptide of [1], wherein the peptide comprises an amino acid sequence selected from the group consisting of SEQ ID NOs: 1, 6, 11, 12, 16, 20, 21, 22, 28, 29, 32, 37, 38, 39, 44 and 59.

[3] An isolated peptide comprising an amino acid sequence in which 1, 2, or several amino acid(s) are substituted, deleted, inserted, and/or added in the amino acid sequence selected from the group consisting of SEQ ID NOs: 1, 6, 11, 12, 16, 20, 21, 22, 28, 29, 32, 37, 38, 39, 44 and 59, and wherein the peptide has cytotoxic T lymphocyte (CTL) inducibility.

[4] The isolated peptide of any one of [1] to [3], wherein said peptide binds to HLA antigen.

[5] The isolated peptide of [4], wherein said HLA antigen is HLA-A24 or HLA-A2.

[6] The peptide of [5], wherein the peptide has one or both of the following characteristics:

(a) the second amino acid from the N-terminus of the amino acid sequence selected from the group consisting of SEQ ID NOs: 1, 6, 11, 12, 16, 20, 21, 22, 28, 29, 32, and 37 is selected from the group consisting of phenylalanine, tyrosine, methionine, or tryptophan, and

(b) the C-terminal amino acid of the amino acid sequence selected from the group consisting of SEQ ID NOs: 1, 6, 11, 12, 16, 20, 21, 22, 28, 29, 32 and 37 is selected from the group consisting of phenylalanine, leucine, isoleucine, tryptophan, or methionine.

[7] The peptide of [5], wherein the peptide has one or both of the following characteristics:

(a) the second amino acid from the N-terminus of the amino acid sequence selected from the group consisting of SEQ ID NOs: 38, 39, 44 and 59 is selected from the group consisting of leucine and methionine; and

(b) the C-terminal amino acid of the amino acid sequence selected from the group consisting of SEQ ID NOs: 38, 39, 44 and 59 is selected from the group consisting of valine and leucine.

[8] The isolated peptide of any one of [1] to [7], wherein said peptide is nonapeptide or decapeptide.

[9] An isolated polynucleotide encoding the isolated peptide of any one of [1] to [8].

[10] A composition for inducing CTL, wherein the composition comprises one or more of the peptide(s) of any one of [1] to [8], or one or more of the polynucleotide(s) of [9].

[11] A pharmaceutical composition for the treatment and/or prophylaxis of cancer, and/or the prevention of a postoperative recurrence thereof, wherein the composition comprises one or more of the peptide(s) of any one of [1] to [8], or one or more of the polynucleotide(s) of [9].

[12] The pharmaceutical composition of Mt wherein said composition is formulated for the administration to a subject whose HLA antigen is HLA-A24 or A2.

[13] A method for inducing an antigen-presenting cell (APC) with CTL inducibility that comprises the step of selected from the group consisting of:

(a) contacting an APC with a peptide of any one of [1] to [8] in vitro, ex vivo or in vivo, and

(b) introducing a polynucleotide encoding the peptide of any one of [1] to [8] into an APC.

[14] A method for inducing CTL that comprises a step selected from the group consisting of:

(a) co-culturing CD8 positive T cells with APCs that present on the surface a complex of an HLA antigen and the peptide of any one of [1] to [8],

(b) co-culturing CD8 positive T cells with exosomes that present on the surface a complex of an HLA antigen and the peptide of any one of [1] to [8], and

(c) introducing a gene that comprises a polynucleotide encoding a T cell receptor (TCR) subunit polypeptide bound to a peptide of any one of [1] to [8] into a T cell.

[15] An isolated APC that presents on its surface a complex of an HLA antigen and the peptide of any one of [1] to [8].

[16] The APC of [15], which is induced by the method of [13].

[17] An isolated CTL that targets the peptide of any one of [1] to [8].

[18] A CTL of [17] induced by the method of [14].

[19] A method of inducing immune response against cancer in a subject in need thereof, said method comprising the step of administering to the subject a composition comprising the peptide of any one of [1] to [8], an immunologically active fragment thereof, or a polynucleotide encoding the peptide or the fragment.

[20] An antibody or immunologically active fragment thereof against the peptides of any one of [1] to [8].

[21] A vector comprising a nucleotide sequence encoding the peptides of any one of [1] to [8].

[22] A host cell transformed or transfected with an expression vector according to [21].

[23] A diagnostic kit comprising the peptides of any one of [1] to [8], the nucleotide of [9] or the antibody of [20].

[24] The isolated peptide of any one of [1] to [8] selected from the group consisting of SEQ ID NOs: 1, 6, 11, 12, 16, 20, 21, 22, 28, 29, 32, 37, 38, 39, 44 and 59.

Alternatively, in another embodiment, the present invention also provides following peptides and use thereof:

[1] An isolated peptide having CTL inducibility, wherein the peptide consists of the amino acid sequence of TTLL4 or an immunologically active fragment thereof, or an isolated peptide having CTL inducibility, wherein the peptide comprises or consists of an amino acid sequence of an immunologically active fragment of the peptide consisting of the amino acid sequence of SEQ ID NO: 80.

[2] The isolated peptide of [1], wherein the peptide comprises an amino acid sequence selected from the group consisting of SEQ ID NOs: 1, 3 to 37 and 38 to 73.

[3] An isolated peptide of [1] or [2] in which 1, 2, or several amino acid(s) are substituted, inserted, deleted, or added to yield a modified peptide that retains the CTL inducibility of the original peptide.

[4] The isolated peptide of [1] to [3], wherein said peptide binds to HLA antigen.

[5] The isolated peptide of [4], wherein said HLA antigen is HLA-A24 or HLA-A2.

[6] The isolated peptide of [3] to [5], wherein, in the context of HLA-A24, the peptide has one or both of the following characteristics:

(a) the second amino acid from the N-terminus is or is modified to be an amino acid selected from the group consisting of phenylalanine, tyrosine, methionine, or tryptophan, and

(b) the C-terminal amino acid is or is modified to be an amino acid selected from the group consisting of phenylalanine, leucine, isoleucine, tryptophan, or methionine.

[7] The isolated peptide of [3] to [5], wherein, in the context of HLA-A2, the peptide has at least one substitution selected from the group consisting of:

(a) the second amino acid from the N-terminus is selected from the group consisting of leucine and methionine; and

(b) the C-terminal amino acid is selected from the group consisting of valine and leucine.

[8] The isolated peptide of any one of [1] to [7], wherein said peptide is nonapeptide or decapeptide.

Although any methods and materials similar or equivalent to those described herein can be used in the practice or testing of embodiments of the present invention, the preferred methods, devices, and materials are now described. However, before the present materials and methods are described, it should be understood that these descriptions are merely illustrative and not intended to be limited. It should also be understood that the present invention is not limited to the particular sizes, shapes, dimensions, materials, methodologies, protocols, etc. described herein, as these may vary in accordance with routine experimentation and optimization. Furthermore, the terminology used in the description is for the purpose of describing the particular versions or embodiments only, and is not intended to limit the scope of the present invention which will be limited only by the appended claims.

The disclosure of each publication, patent or patent application mentioned in this specification is specifically incorporated by reference herein in its entirety. However, nothing herein is to be construed as an admission that the invention is not entitled to antedate such disclosure by virtue or prior invention.

Unless otherwise defined, all technical and scientific terms used herein have the same meaning as commonly understood by one of ordinary skill in the art to which the present invention belongs. In case of conflict, the present specification, including definitions, will control. In addition, the materials, methods, and examples are illustrative only and not intended to be limiting.

### I. DEFINITIONS

The words “a”, “an”, and “the” as used herein mean “at least one” unless otherwise specifically indicated.

The terms “isolated” and “purified” used in relation with a substance (e.g., peptide, antibody, polynucleotide, etc.) indicates that the substance is substantially free from at least one substance that may else be included in the natural source. Thus, an isolated or purified peptide refers to peptide that are substantially free of cellular material such as carbohydrate, lipid, or other contaminating proteins from the cell or tissue source from which the peptide is derived, or substantially free of chemical precursors or other chemicals when chemically synthesized. The term “substantially free of cellular material” includes preparations of a peptide in which the peptide is separated from cellular components of the cells from which it is isolated or recombinantly produced. Thus, a peptide that is substantially free of cellular material includes preparations of polypeptide having less than about 30%, 20%, 10%, or 5% (by dry weight) of heterologous protein (also referred to herein as a “contaminating protein”). When the peptide is recombinantly produced, it is also preferably substantially free of culture medium, which includes preparations of peptide with culture medium less than about 20%, 10%, or 5% of the volume of the peptide preparation. When the peptide is produced by chemical synthesis, it is preferably substantially free of chemical precursors or other chemicals, which includes preparations of peptide with chemical precursors or other chemicals involved in the synthesis of the peptide less than about 30%, 20%, 10%, 5% (by dry weight) of the volume of the peptide preparation. That a particular peptide preparation contains an isolated or purified peptide can be shown, for example, by the appearance of a single band following sodium dodecyl sulfate (SDS)-polyacrylamide gel electrophoresis of the protein preparation and Coomassie Brilliant Blue staining or the like of the gel. In a preferred embodiment, peptides and polynucleotides of the present invention are isolated or purified.

The terms “polypeptide”, “peptide” and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers.

The term “oligopeptide” sometimes used in the present specification is used to refer to peptides of the present invention which are 20 residues or fewer, typically 15 residues or fewer in length and is typically composed of between about 8 and about 11 residues, often 9 or 10 residues. The latter are referred to herein as “nonapeptides” and “decapeptides”, respectively.

The term “amino acid” as used herein refers to naturally occurring and synthetic amino acids, as well as amino acid analogs and amino acid mimetics that similarly function to the naturally occurring amino acids. Naturally occurring amino acids are those encoded by the genetic code, as well as those modified after translation in cells (e.g., hydroxyproline, gamma-carboxyglutamate, and O-phosphoserine). The phrase “amino acid analog” refers to compounds that have the same basic chemical structure (an alpha carbon bound to a hydrogen, a carboxy group, an amino group, and an R group) as a naturally occurring amino acid but have a modified R group or modified backbones (e.g., homoserine, norleucine, methionine, sulfoxide, methionine methyl sulfonium). The phrase “amino acid mimetic” refers to chemical compounds that have different structures but similar functions to general amino acids.

Amino acids may be referred to herein by their commonly known three letter symbols or the one-letter symbols recommended by the IUPAC-IUB Biochemical Nomenclature Commission.

The terms “gene”, “polynucleotide”, “oligonucleotide”, “nucleotide” and “nucleic acid” are used interchangeably herein and, unless otherwise specifically indicated, are referred to by their commonly accepted single-letter codes.

The term “agent”, and “composition” are used interchangeably herein to refer to a product including the specified ingredients in the specified amounts, as well as any product which results, directly or indirectly, from combination of the specified ingredients in the specified amounts. Such term in relation to pharmaceutical composition, is intended to encompass a product including the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically or physiologically acceptable carrier.

The term “active ingredient” herein refers to a substance in an agent or composition that is biologically or physiologically active. Particularly, in the context of pharmaceutical agent or composition, the term “active ingredient” refers to a substance that shows an objective pharmacological effect. For example, in case of pharmaceutical agents or compositions for use in the treatment or prevention of cancer, active ingredients in the agents or compositions may lead to at least one biological or physiologically action on cancer cells and/or tissues directly or indirectly. Preferably, such action may include reducing or inhibiting cancer cell growth, damaging or killing cancer cells and/or tissues, and so on. Typically, indirect effect of active ingredients is inductions of CTLs recognizing or killing cancer cells. Before being formulated, the “active ingredient” may also be referred to as “bulk”, “drug substance” or “technical product”.

The phrase “pharmaceutically acceptable carrier” or “physiologically acceptable carrier”, as used herein, means a pharmaceutically or physiologically acceptable material, composition, substance or vehicle, including, but are not limited to, a liquid or solid filler, diluent, excipient, solvent or encapsulating material.

Some pharmaceutical agents or compositions of the present invention find particular use as vaccines. In the context of the present invention, the phrase “vaccine” (also referred to as an “immunogenic composition”) refers to an agent or composition that has the function to improve, enhance and/or induce anti-tumor immunity upon inoculation into animals.

Unless otherwise defined, the term “cancer” refers to the cancers or tumors that over-express the TTLL4 gene, examples of which include, but are not limited to, bladder cancer, cholangiocellular carcinoma, chronic myelogenous leukemia (CML), colon and rectum cancer, esophageal cancer, liver cancer, lymphoma, pancreatic cancer, prostate cancer, renal carcinoma, small-cell lung cancer (SCLC), non-small-cell lung cancer (NSCLC), soft tissue tumor and osteosarcoma.

Unless otherwise defined, the terms “cytotoxic T lymphocyte”, “cytotoxic T cell” and “CTL” are used interchangeably herein and unless otherwise specifically indicated, refer to a sub-group of T lymphocytes that are capable of recognizing non-self cells (e.g., tumor/cancer cells, virus-infected cells) and inducing the death of such cells.

Unless otherwise defined, the terms “HLA-A24” refers to the HLA-A24 type containing the subtypes, examples of which include, but are not limited to, HLA-A*2401, HLA-A*2402, HLA-A*2403, HLA-A*2404, HLA-A*2407, HLA-A*2408, HLA-A*2420, HLA-A*2425 and HLA-A*2488.

Unless otherwise defined, the term “HLA-A2”, as used herein, representatively refers to the subtypes, examples of which include, but are not limited to, HLA-A*0201, HLA-A*0202, HLA-A*0203, HLA-A*0204, HLA-A*0205, HLA-A*0206, HLA-A*0207, HLA-A*0210, HLA-A*0211, HLA-A*0213, HLA-A*0216, HLA-A*0218, HLA-A*0219, HLA-A*0228 and HLA-A*0250.

Unless otherwise defined, the term “kit” as used herein, is used in reference to a combination of reagents and other materials. It is contemplated herein that the kit may include microarray, chip, marker, and so on. It is not intended that the term “kit” be limited to a particular combination of reagents and/or materials.

As used herein, in the context of a subject or patient, the phrase “subject's (or patient's) HLA antigen is HLA A24 or HLA-A2” refers to that the subject or patient homozygously or heterozygously possess HLA-A24 or HLA-A2 antigen gene as the MHC (major histocompatibility complex) Class I molecule, and HLA-A24 or HLA-A2 antigen is expressed in cells of the subject or patient as an HLA antigen.

To the extent that the methods and compositions of the present invention find utility in the context of the “treatment” of cancer, a treatment is deemed “efficacious” if it leads to clinical benefit such as, reduction in expression of TTLL4 gene, or a decrease in size, prevalence, or metastatic potential of the cancer in the subject. When the treatment is applied prophylactically, “efficacious” means that it retards or prevents cancers from forming or prevents or alleviates a clinical symptom of cancer. Efficaciousness is determined in association with any known method for diagnosing or treating the particular tumor type.

To the extent that the methods and compositions of the present invention find utility in the context of the “prevention” and “prophylaxis” of cancer, such terms are interchangeably used herein to refer to any activity that reduces the burden of mortality or morbidity from disease. Prevention and prophylaxis can occur “at primary, secondary and tertiary prevention levels.” While primary prevention and prophylaxis avoid the development of a disease, secondary and tertiary levels of prevention and prophylaxis encompass activities aimed at the prevention and prophylaxis of the progression of a disease and the emergence of symptoms as well as reducing the negative impact of an already established disease by restoring function and reducing disease-related complications. Alternatively, prevention and prophylaxis can include a wide range of prophylactic therapies aimed at alleviating the severity of the particular disorder, e.g. reducing the proliferation and metastasis of tumors.

In the context of the present invention, the treatment and/or prophylaxis of cancer and/or the prevention of postoperative recurrence thereof include any of the following steps, such as the surgical removal of cancer cells, the inhibition of the growth of cancerous cells, the involution or regression of a tumor, the induction of remission and suppression of occurrence of cancer, the tumor regression, and the reduction or inhibition of metastasis. Effective treatment and/or the prophylaxis of cancer decreases mortality and improves the prognosis of individuals having cancer, decreases the levels of tumor markers in the blood, and alleviates detectable symptoms accompanying cancer. For example, reduction or improvement of symptoms constitutes effectively treating and/or the prophylaxis include 10%, 20%, 30% or more reduction, or stable disease.

In the context of the present invention, the term “antibody” refers to immunoglobulins and fragments thereof that are specifically reactive to a designated protein or peptide thereof. An antibody can include human antibodies, primatized antibodies, chimeric antibodies, bispecific antibodies, humanized antibodies, antibodies fused to other proteins or radiolabels, and antibody fragments. Furthermore, an antibody herein is used in the broadest sense and specifically covers intact monoclonal antibodies, polyclonal antibodies, multispecific antibodies (e.g., bispecific antibodies) formed from at least two intact antibodies, and antibody fragments so long as they exhibit the desired biological activity. An “antibody” indicates all classes (e.g., IgA, IgD, IgE, IgG and IgM).

Unless otherwise defined, all technical and scientific terms used herein have the same meaning as commonly understood by one of ordinary skill in the art to which this invention belongs.

### II. PEPTIDES

Peptides of the present invention described in detail below may be referred to as “TTLL4 peptide(s)” or “TTLL4 polypeptide(s)”.

To demonstrate that peptides derived from TTLL4 function as an antigen recognized by CTLs, peptides derived from TTLL4 (SEQ ID NO: 80) were analyzed to determine whether they were antigen epitopes restricted by HLA-A24 or A2 which are commonly encountered HLA alleles (Date Y et al., Tissue Antigens 47: 93-101, 1996; Kondo A et al., J Immunol 155: 4307-12, 1995; Kubo R T et al., J Immunol 152: 3913-24, 1994).

Candidates of HLA-A24 binding peptides derived from TTLL4 were identified based on their binding affinities to HLA-A24. The following candidate peptides were identified:

(SEQ ID NO: 1)
TTLL4-A24-9-750,
(SEQ ID NO: 3)
TTLL4-A24-9-994,
(SEQ ID NO: 4)
TTLL4-A24-9-769,
(SEQ ID NO: 5)
TTLL4-A24-9-755,
(SEQ ID NO: 6)
TTLL4-A24-9-79,
(SEQ ID NO: 7)
TTLL4-A24-9-684,
(SEQ ID NO: 8)
TTLL4-A24-9-689,
(SEQ ID NO: 9)
TTLL4-A24-9-779,
(SEQ ID NO: 10)
TTLL4-A24-9-304,
(SEQ ID NO: 11)
TTLL4-A24-9-793,
(SEQ ID NO: 12)
TTLL4-A24-9-691,
(SEQ ID NO: 13)
TTLL4-A24-9-41,
(SEQ ID NO: 14)
TTLL4-A24-9-1086,
(SEQ ID NO: 15)
TTLL4-A24-9-1186,
(SEQ ID NO: 16)
TTLL4-A24-9-103,
(SEQ ID NO: 17)
TTLL4-A24-9-362,
(SEQ ID NO: 18)
TTLL4-A24-9-1037,
(SEQ ID NO: 19)
TTLL4-A24-9-773,
(SEQ ID NO: 20)
TTLL4-A24-10-103,
(SEQ ID NO: 21)
TTLL4-A24-10-773,
(SEQ ID NO: 22)
TTLL4-A24-10-883,
(SEQ ID NO: 23)
TTLL4-A24-10-127,
(SEQ ID NO: 24)
TTLL4-A24-10-684,
(SEQ ID NO: 25)
TTLL4-A24-10-1043,
(SEQ ID NO: 26)
TTLL4-A24-10-223,
(SEQ ID NO: 27)
TTLL4-A24-10-122,
(SEQ ID NO: 28)
TTLL4-A24-10-1186,
(SEQ ID NO: 29)
TTLL4-A24-10-1022,
(SEQ ID NO: 30)
TTLL4-A24-10-689,
(SEQ ID NO: 31)
TTLL4-A24-10-804,
(SEQ ID NO: 32)
TTLL4-A24-10-994,
(SEQ ID NO: 33)
TTLL4-A24-10-993,
(SEQ ID NO: 34)
TTLL4-A24-10-1105,
(SEQ ID NO: 35)
TTLL4-A24-10-696,
(SEQ ID NO: 36)
TTLL4-A24-10-665,
(SEQ ID NO: 37)
TTLL4-A24-10-891,
(SEQ ID NO: 38)
TTLL4-A24-10-254
and
(SEQ ID NO: 39)
TTLL4-A24-10-194..

Moreover, after in vitro stimulation of T-cells by dendritic cells (DCs) loaded with these peptides, CTLs were successfully established using each of the following peptides:

(SEQ ID NO: 1)
TTLL4-A24-9-750,
(SEQ ID NO: 6)
TTLL4-A24-9-79,
(SEQ ID NO: 11)
TTLL4-A24-9-793,
(SEQ ID NO: 12)
TTLL4-A24-9-691,
(SEQ ID NO: 16)
TTLL4-A24-9-103,
(SEQ ID NO: 20)
TTLL4-A24-10-103,
(SEQ ID NO: 21)
TTLL4-A24-10-773,
(SEQ ID NO: 22)
TTLL4-A24-10-883,
(SEQ ID NO: 28)
TTLL4-A24-10-1186,
(SEQ ID NO: 29)
TTLL4-A24-10-1022,
(SEQ ID NO: 32)
TTLL4-A24-10-994
and
(SEQ ID NO: 37)
TTLL4-A24-10-891.

Candidates of HLA-A2 binding peptides derived from TTLL4 were identified based on their binding affinities to HLA-A2. The following peptides are considered to be candidate peptides for immunotherapy:

(SEQ ID NO: 38)
TTLL4-A2-9-222,
(SEQ ID NO: 39)
TTLL4-A2-9-805,
(SEQ ID NO: 40)
TTLL4-A2-9-610,
(SEQ ID NO: 41)
TTLL4-A2-9-1163,
(SEQ ID NO: 42)
TTLL4-A2-9-575,
(SEQ ID NO: 43)
TTLL4-A2-9-1189,
(SEQ ID NO: 44)
TTLL4-A2-9-66,
(SEQ ID NO: 45)
TTLL4-A2-9-864,
(SEQ ID NO: 46)
TTLL4-A2-9-899,
(SEQ ID NO: 47)
TTLL4-A2-9-147,
(SEQ ID NO: 48)
TTLL4-A2-9-578,
(SEQ ID NO: 49)
TTLL4-A2-9-697,
(SEQ ID NO: 50)
TTLL4-A2-9-1088,
(SEQ ID NO: 51)
TTLL4-A2-9-988,
(SEQ ID NO: 52)
TTLL4-A2-9-423,
(SEQ ID NO: 53)
TTLL4-A2-9-852,
(SEQ ID NO: 54)
TTLL4-A2-9-128,
(SEQ ID NO: 55)
TTLL4-A2-9-107,
(SEQ ID NO: 56)
TTLL4-A2-9-605,
(SEQ ID NO: 57)
TTLL4-A2-9-356,
(SEQ ID NO: 58)
TTLL4-A2-10-363,
(SEQ ID NO: 59)
TTLL4-A2-10-574,
(SEQ ID NO: 60)
TTLL4-A2-10-895,
(SEQ ID NO: 61)
TTLL4-A2-10-605,
(SEQ ID NO: 62)
TTLL4-A2-10-578,
(SEQ ID NO: 63)
TTLL4-A2-10-756,
(SEQ ID NO: 64)
TTLL4-A2-10-550,
(SEQ ID NO: 65)
TTLL4-A2-10-610,
(SEQ ID NO: 66)
TTLL4-A2-10-107,
(SEQ ID NO: 67)
TTLL4-A2-10-933,
(SEQ ID NO: 68)
TTLL4-A2-10-1163,
(SEQ ID NO: 69)
TTLL4-A2-10-871,
(SEQ ID NO: 70)
TTLL4-A2-10-863,
(SEQ ID NO: 71)
TTLL4-A2-10-852,
(SEQ ID NO: 72)
TTLL4-A2-10-62,
(SEQ ID NO: 73)
TTLL4-A2-10-804,
(SEQ ID NO: 74)
TTLL4-A2-10-70,
(SEQ ID NO: 75)
TTLL4-A2-10-1092,
(SEQ ID NO: 76)
TTLL4-A2-10-1113,
(SEQ ID NO: 77)
TTLL4-A2-10-778
and
(SEQ ID NO: 78)
TTLL4-A2-10-86.

Moreover, after in vitro stimulation of T-cells by dendritic cells (DCs) pulsed (loaded) with these peptides, CTLs were successfully established using each of the following peptides:

(SEQ ID NO: 38)
TTLL4-A02-9-222,
(SEQ ID NO: 39)
TTLL4-A02-9-805,
(SEQ ID NO: 44)
TTLL4-A02-9-66
and
(SEQ ID NO: 59)
TTLL4-A02-10-574.

These established CTLs show potent specific CTL activity against target cells pulsed with respective peptides. These results herein demonstrate that TTLL4 is an antigen recognized by CTL and that the peptides are epitope peptides of TTLL4 restricted by HLA-A24 or HLA-A2.

Since the TTLL4 gene is over-expressed in cancer cells and tissues, including for example those of bladder cancer, cholangiocellular carcinoma, CML, colon and rectum cancer, esophageal cancer, liver cancer, lymphoma, pancreatic cancer, prostate cancer, renal carcinoma, SCLC, NSCLC, soft tissue tumor and osteosarcoma, and not expressed in most normal organs, it represents a good target for immunotherapy. Thus, the present invention provides nonapeptides (peptides composed of nine amino acid residues) and decapeptides (peptides composed of ten amino acid residues) corresponding to CTL-recognized epitopes from TTLL4. Particularly preferred examples of nonapeptides and decapeptides of the present invention include those peptides having an amino acid sequence selected from among SEQ ID NOs: 1, 3 to 37 and 38 to 73.

Generally, software programs now available, for example, on the Internet, such as those described in Parker K C et al., J Immunol 1994, 152(1): 163-75 and Nielsen M et al., Protein Sci 2003; 12: 1007-17 can be used to calculate the binding affinities between various peptides and HLA antigens in silico. Binding affinity with HLA antigens can be measured as described, for example, in Parker K C et al., J Immunol 1994, 152(1): 163-75, Kuzushima K et al., Blood 2001, 98(6): 1872-81, Larsen M V et al. BMC Bioinformatics. 2007; 8: 424, Buus S et al. Tissue Antigens., 62:378-84, 2003, Nielsen M et al., Protein Sci 2003; 12: 1007-17, and Nielsen M et al. PLoS ONE 2007; 2: e796, which are summarized in, e.g., Lafuente E M et al., Current Pharmaceutical Design, 2009, 15, 3209-3220. Methods for determining binding affinity are described, for example, in the Journal of Immunological Methods (1995, 185: 181-190) and Protein Science (2000, 9: 1838-1846). Therefore, one can readily utilize such software programs to select those fragments derived from TTLL4 that have high binding affinity with HLA antigens using such software programs. Accordingly, the present invention encompasses peptides composed of any fragments derived from TTLL4, which would be determined to bind with HLA antigens by such known programs. Furthermore, such peptides may include the peptide consisting of the full length of TTLL4 sequence.

The peptides of the present invention, particularly the nonapeptides and decapeptides of the present invention, can be flanked with additional amino acid residues, so long as the resulting peptide retains its CTL inducibility. The particular additional amino acid residues can be composed of any kind of amino acids, so long as they do not impair the CTL inducibility of the original peptide. Thus, the present invention encompasses peptides having a binding affinity for HLA antigens, in particular peptides derived from TTLL4. Such peptides are, for example, less than about 40 amino acids, often less than about 20 amino acids, and usually less than about 15 amino acids.

In general, the modification of one, two or more amino acids in a peptide will not influence the function of the peptide, and in some cases will even enhance the desired function of the original protein. In fact, modified peptides (i.e., peptides composed of an amino acid sequence, in which 1, 2 or several amino acid residues have been modified (i.e., substituted, added, deleted or inserted) as compared to an original reference sequence) have been known to retain the biological activity of the original peptide (Mark et al., Proc Natl Acad Sci USA 1984, 81: 5662-6; Zoller and Smith, Nucleic Acids Res 1982, 10: 6487-500; Dalbadie-McFarland et al., Proc Natl Acad Sci USA 1982, 79: 6409-13). Thus, in one embodiment, the peptides of the present invention have both CTL inducibility and an amino acid sequence selected from among SEQ ID NOs: 1, 3 to 37 and 38 to 73, in which one, two or even more amino acids are added and/or substituted.

Those of skill in the art will recognize that individual modifications (i.e., deletions, insertions, additions and/or substitutions) to an amino acid sequence that alter a single amino acid or a small percentage of the overall amino acid sequence tend to result in the conservation of the properties of the original amino acid side-chain. As such, they are often referred to as “conservative substitutions” or “conservative modifications”, wherein the alteration of a protein results in a modified protein having a function analogous to the original protein. Conservative substitution tables providing functionally similar amino acids are well known in the art. Examples of amino acid side-chains characteristics that are desirable to conserve include, for example: hydrophobic amino acids (A, I, L, M, F, P, W, Y, V), hydrophilic amino acids (R, D, N, C, E, Q, G, H, K, S, T), and side-chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). In addition, the following eight groups each contain amino acids that are accepted in the art as conservative substitutions for one another:

1) Alanine (A), Glycine (G);

2) Aspartic acid (D), Glutamic acid (E);

3) Aspargine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M) (see, e.g., Creighton, Proteins 1984).

Such conservatively modified peptides are also considered to be peptides of the present invention. However, peptides of the present invention are not restricted thereto and can include non-conservative modifications, so long as the resulting modified peptide retains the CTL inducibility of the original unmodified peptide. Furthermore, modified peptides should not exclude CTL inducible peptides derived from polymorphic variants, interspecies homologues, and alleles of TTLL4.

Amino acid residues may be inserted, substituted or added to the peptides of the present invention or, alternatively, amino acid residues may be deleted therefrom to achieve a higher binding affinity. To retain the requisite CTL inducibility, one preferably modifies (i.e, deletes, inserts, adds and/or substitutes) only a small number (for example, 1, 2 or several) or a small percentage of amino acids. Herein, the term “several” means 5 or fewer amino acids, for example, 4 or 3 or fewer. The percentage of amino acids to be modified is preferably 20% or less, more preferably 15% of less, and even more preferably 10% or less, for example 1 to 5%.

When used in the context of immunotherapy, the peptides of the present invention should be presented on the surface of a cell or exosome, preferably as a complex with an HLA antigen. Therefore, it is preferable to select peptides that not only induce CTLs but also possess high binding affinity to the HLA antigen. To that end, the peptides can be modified by substitution, insertion, deletion and/or addition of the amino acid residues to yield a modified peptide having improved binding affinity. In addition to peptides that are naturally displayed, since the regularity of the sequences of peptides displayed by binding to HLA antigens is already known (J Immunol 1994, 152: 3913; Immunogenetics 1995, 41: 178; J Immunol 1994, 155: 4307), modifications based on such regularity can be introduced into the immunogenic peptides of the invention.

For example, peptides possessing high HLA-A24 binding affinity tend to have the second amino acid from the N-terminus substituted with phenylalanine, tyrosine, methionine, or tryptophan. Likewise, peptides in which the C-terminal amino acid is substituted with phenylalanine, leucine, isoleucine, tryptophan, or methionine. Accordingly, it may be desirable to substitute the second amino acid from the N-terminus with phenylalanine, tyrosine, methionine, or tryptophan, and/or the amino acid at the C-terminus with leucine, isoleucine, tryptophan, or methionine in order to increase the HLA-A24 binding affinity. Thus, peptides having an amino acid sequence selected from among SEQ ID NOs: 1 and 3 to 37, wherein the second amino acid from the N-terminus of the amino acid sequence of the SEQ ID NO is substituted with leucine or methionine, and/or wherein the C-terminus of the amino acid sequence of the SEQ ID NO is substituted with valine or leucine are encompassed by the present invention.

Likewise, peptides showing high HLA-A2 binding affinity tend to have the second amino acid from the N-terminus substituted with leucine or methionine and/or the amino acid at the C-terminus substituted with valine or leucine. Alternatively, it may be desirable to substitute the second amino acid from the N-terminus with leucine or methionine, and/or the amino acid at the C-terminus with valine or leucine in order to increase the HLA-A2 binding affinity. Thus, peptides having an amino acid sequence selected from among SEQ ID NOs: 38 to 73, wherein the second amino acid from the N-terminus of the amino acid sequence of the SEQ ID NO is substituted with phenylalanine, tyrosine, methionine, or tryptophan, and/or wherein the C-terminus of the amino acid sequence of the SEQ ID NO is substituted with phenylalanine, leucine, isoleucine, tryptophan, or methionine are encompassed by the present invention.

Substitutions can be introduced not only at the terminal amino acids but also at the position of potential T cell receptor (TCR) recognition of peptides. Several studies have demonstrated that a peptide with amino acid substitutions can be equal to or better than the original, for example CAP 1, p53_{(264-272),} Her-2/neu₍₃₆₉₋₃₇₇₎ or gp100₍₂₀₉₋₂₁₇₎ (Zaremba et al. Cancer Res. 57, 4570-4577, 1997, T. K. Hoffmann et al. J. Immunol. (2002);168(3):1338-47., S. O. Dionne et al. Cancer Immunol immunother. (2003) 52: 199-206 and S. O. Dionne et al. Cancer Immunology, Immunotherapy (2004) 53, 307-314).

The present invention also contemplates the addition of 1, 2 or several amino acids can also be added to the N and/or C-terminus of the present peptides. Such modified peptides having high HLA antigen binding affinity and retained CTL inducibility are also included in the present invention.

For example, the present invention provides an isolated peptide of less than 14, 13, 12, 11, or 10 amino acids in length, which has CTL inducibility and comprises the amino acid sequence selected from the group consisting of:

(i) an amino acid sequence in which 1, 2 or several amino acid(s) are modified in the amino acid sequence selected from the group consisting of SEQ ID NOs: 1 to 19 and 38-57, wherein the peptide binds an HLA antigen and induces cytotoxic T lymphocytes,

(ii) the amino acid sequence of (i), wherein, in the context of HLA-A24, the amino acid sequence has one or both of the following characteristics:

(a) the second amino acid from the N-terminus of said SEQ ID NOs is or is modified to be an amino acid selected from the group consisting of phenylalanine, tyrosine, methionine, and tryptophan, and

(b) the C-terminal amino acid of said SEQ ID NOs is or is modified to be an amino acid selected from the group consisting of phenylalanine, leucine, isoleucine, tryptophan, and methionine, and

(iii) the amino acid sequence of (i), wherein, in the context of HLA-A2, the amino acid sequence has one or both of the following characteristics:

(a) the second amino acid from the N-terminus of said SEQ ID NO is or is modified to be an amino acid selected from the group consisting of leucine and methionine; and

(b) the C-terminal amino acid of said SEQ ID NO is or is modified to be an amino acid selected from the group consisting of valine and leucine.

Moreover, the present invention also provides an isolated peptide of less than 15, 14, 13, 12, or 11 amino acids in length, which has CTL inducibility and comprises the amino acid sequence selected from the group consisting of:

(i′) an amino acid sequence in which 1, 2 or several amino acid(s) are modified in the amino acid sequence selected from the group consisting of SEQ ID NOs: 20 to 37 and 58-78, wherein the peptide binds an HLA antigen and induces cytotoxic T lymphocytes,

(ii′) the amino acid sequence of (i′), wherein, in the context of HLA-A24, the amino acid sequence has one or both of the following characteristics:

(a) the second amino acid from the N-terminus of said SEQ ID NOs is or is modified to be an amino acid selected from the group consisting of phenylalanine, tyrosine, methionine, and tryptophan, and

(b) the C-terminal amino acid of said SEQ ID NOs is or is modified to be an amino acid selected from the group consisting of phenylalanine, leucine, isoleucine, tryptophan, and methionine.

(iii′) the amino acid sequence of (i′), wherein, in the context of HLA-A2, the amino acid sequence has one or both of the following characteristics:

(a) the second amino acid from the N-terminus of said SEQ ID NOs is or is modified to be an amino acid selected from the group consisting of leucine and methionine; and

(b) the C-terminal amino acid of said SEQ ID NOs is or is modified to be an amino acid selected from the group consisting of valine and leucine.

These peptides are processed in APC to present a peptide of (i), (ii), (iii), (i′), (ii′), and (iii′) thereon, when these peptides are contacted with, or introduced in APC.

However, when the peptide sequence is identical to a portion of the amino acid sequence of an endogenous or exogenous protein having a different function, side effects such as autoimmune disorders and/or allergic symptoms against specific substances may be induced. Therefore, it is preferable to first perform homology searches using available databases to avoid situations in which the sequence of the peptide matches the amino acid sequence of another protein. When it becomes clear from the homology searches that there exists not even a peptide with 1 or 2 amino acid differences as compared to the objective peptide, the objective peptide can be modified in order to increase its binding affinity with HLA antigens, and/or increase its CTL inducibility without any danger of such side effects.

Although peptides having high binding affinity to the HLA antigens as described above are expected to be highly effective, the candidate peptides, which are selected according to the presence of high binding affinity as an indicator, are further examined for the presence of CTL inducibility. Herein, the phrase “CTL inducibility” indicates the ability of the peptide to induce cytotoxic T lymphocytes (CTLs) when presented on antigen-presenting cells (APCs). Further, “CTL inducibility” includes the ability of the peptide to induce CTL activation, CTL proliferation, promote lysis of target cells by CTL, and to increase IFN-gamma production by CTL.

Confirmation of CTL inducibility is accomplished by inducing APCs carrying human MHC antigens (for example, B-lymphocytes, macrophages, and dendritic cells (DCs)), or more specifically DCs derived from human peripheral blood mononuclear leukocytes, and after stimulation of APCs with a test peptides, mixing APCs with CD8 positive cells to induce CTLs, and then measuring the IFN-gamma produced and released by CTL against the target cells. As the reaction system, transgenic animals that have been produced to express a human HLA antigen (for example, those described in BenMohamed L, Krishnan R, Longmate J, Auge C, Low L, Primus J, Diamond D J, Hum Immunol 2000, 61(8): 764-79, Related Articles, Books, Linkout Induction of CTL response by a minimal epitope vaccine in HLA A*0201/DR1 transgenic mice: dependence on HLA class II restricted T(H) response) can be used. Alternatively, the target cells can be radiolabeled with ⁵¹Cr and such, and cytotoxic activity of CTL can be calculated from radioactivity released from the target cells. Alternatively, CTL inducibility can be assessed by measuring IFN-gamma produced and released by CTL in the presence of APCs that carry immobilized peptides, and visualizing the inhibition zone on the media using anti-IFN-gamma monoclonal antibodies.

As a result of examining the CTL inducibility of the peptides as described above, it was discovered that nonapeptides or decapeptides selected from among the amino acid sequences indicated by SEQ ID NOs: 1, 6, 11, 12, 16, 20, 21, 22, 28, 29, 32, 37, 38, 39, 44 and 59 showed particularly high CTL inducibility as well as high binding affinity to an HLA antigen. Thus, these peptides are exemplified as preferred embodiments of the present invention.

Furthermore, homology analysis results demonstrated that such peptides do not have significant homology with peptides derived from any other known human gene products. Accordingly, the possibility of unknown or undesired immune responses arising when used for immunotherapy is lowered. Therefore, also from this aspect, these peptides are useful for eliciting immunity against TTLL4 in cancer patients. Thus, the preferred peptides of the present invention, preferably, peptides having an amino acid sequence selected from among SEQ ID NOs: 1, 6, 11, 12, 16, 20, 21, 22, 28, 29, 32, 37, 38, 39, 44 and 59 are encompassed by the present invention.

In addition to the above-described modifications, the peptides of the present invention can also be linked to other peptides, so long as the resulting linked peptide retains the requisite CTL inducibility of the original peptide, and more preferably also retains the requisite HLA binding. Examples of suitable “other” peptides include: the peptides of the present invention or the CTL-inducible peptides derived from other TAAs. The peptide of the present invention can be linked “other” peptide via a linker directly or indirectly. Suitable inter-peptide linkers are well known in the art and include, for example AAY (P. M. Daftarian et al., J Trans Med 2007, 5:26), AAA, NKRK (R. P. M. Sutmuller et al., J. Immunol. 2000, 165: 7308-7315) or K (S. Ota et al., Can Res. 62, 1471-1476, K. S. Kawamura et al., J. Immunol. 2002, 168: 5709-5715).

For example, non-TTLL4 tumor associated antigen peptides also can be used substantially simultaneously to increase the immune response via HLA class I and/or class II. It is well established that cancer cells can express more than one tumor associated gene. Thus, it is within the scope of routine experimentation for one of ordinary skill in the art to determine whether a particular subject expresses additional tumor associated genes, and then to include HLA class I and/or HLA class II binding peptides derived from expression products of such genes in TTLL4 compositions or vaccines according to the present invention.

Examples of HLA class I and HLA class II binding peptides are known to those of ordinary skill in the art (for example, see Coulie, Stem Cells 13:393-403, 1995), and can be used in the invention in a like manner as those disclosed herein. Thus, one of ordinary skill in the art can readily prepare polypeptides including one or more TTLL4 peptides and one or more of the non-TTLL4 peptides, or nucleic acids encoding such polypeptides, using standard procedures of molecular biology.

The above described linked peptides are referred to herein as “polytopes”, i.e., groups of two or more potentially immunogenic or immune response stimulating peptides which can be joined together in various arrangements (e.g., concatenated, overlapping). The polytope (or nucleic acid encoding the polytope) can be administered in a standard immunization protocol, e.g., to animals, to test the effectiveness of the polytope in stimulating, enhancing and/or provoking an immune response.

The peptides can be joined together directly or via the use of flanking sequences to form polytopes, and the use of polytopes as vaccines is well known in the art (see, e.g., Thomson et al., Proc. Natl. Acad. Sci. USA 92(13):5845-5849, 1995; Gilbert et al., Nature Biotechnol. 15(12):1280-1284, 1997; Thomson et al., J. Immunol. 157(2):822-826, 1996; Tarn et al., J. Exp. Med. 171(1):299-306, 1990). Polytopes containing various numbers and combinations of epitopes can be prepared and tested for recognition by CTLs and for efficacy in increasing an immune response.

The peptides of the present invention can also be linked to other substances, so long as the resulting linked peptide retains the requisite CTL inducibility of the original peptide. Examples of suitable substances include, for example: peptides, lipids, sugar and sugar chains, acetyl groups, natural and synthetic polymers, etc. The peptides can contain modifications such as glycosylation, side chain oxidation, or phosphorylation, etc., provided the modifications do not destroy the biological activity of the original peptide. These kinds of modifications can be performed to confer additional functions (e.g., targeting function, and delivery function) or to stabilize the peptide.

For example, to increase the in vivo stability of a peptide, it is known in the art to introduce D-amino acids, amino acid mimetics or unnatural amino acids; this concept can also be adapted to the present peptides. The stability of a peptide can be assayed in a number of ways. For instance, peptidases and various biological media, such as human plasma and serum, can be used to test stability (see, e.g., Verhoef et al., Eur J Drug Metab Pharmacokin 1986, 11: 291-302).

Moreover, as noted above, among the modified peptides that are substituted, deleted inserted or added by 1, 2 or several amino acid residues, those having same or higher activity as compared to original peptides can be screened for or selected. The present invention, therefore, also provides the method of screening for or selecting modified peptides having same or higher activity as compared to originals. An illustrative method includes the steps of:

a: substituting, deleting, inserting or adding at least one amino acid residue of a peptide of the present invention,

b: determining the activity of the peptide, and

c: selecting the peptide having same or higher activity as compared to the original.

Herein, the activity to be assayed may include MHC binding activity, APC or CTL inducibility and cytotoxic activity.

### III. PREPARATION OF TTLL4 PEPTIDES

The peptides of the present invention can be prepared using well known techniques. For example, the peptides can be prepared synthetically, using recombinant DNA technology or chemical synthesis. The peptides of the present invention can be synthesized individually or as longer polypeptides including two or more peptides. The peptides can then be isolated i.e., purified or isolated so as to be substantially free of other naturally occurring host cell proteins and fragments thereof, or any other chemical substances.

The peptides of the present invention may contain modifications, such as glycosylation, side chain oxidation, or phosphorylation, provided the modifications do not destroy the biological activity of the original peptide. Other illustrative modifications include incorporation of D-amino acids or other amino acid mimetics that can be used, for example, to increase the serum half life of the peptides.

Peptides of the present invention can be obtained through chemical synthesis based on the selected amino acid sequence. Examples of conventional peptide synthesis methods that can be adapted for the synthesis include:

(i) Peptide Synthesis, Interscience, New York, 1966;

(ii) The Proteins, Vol. 2, Academic Press, New York, 1976;

(iii) Peptide Synthesis (in Japanese), Maruzen Co., 1975;

(iv) Basics and Experiment of Peptide Synthesis (in Japanese), Maruzen Co., 1985;

(v) Development of Pharmaceuticals (second volume) (in Japanese), Vol. 14 (peptide synthesis), Hirokawa, 1991;

(vi) WO99/67288; and

(vii) Barany G. & Merrifield R. B., Peptides Vol. 2, “Solid Phase Peptide Synthesis”, Academic Press, New York, 1980, 100-118.

Alternatively, the present peptides can be obtained adapting any known genetic engineering method for producing peptides (e.g., Morrison J, J Bacteriology 1977, 132: 349-51; Clark-Curtiss & Curtiss, Methods in Enzymology (eds. Wu et al.) 1983, 101: 347-62). For example, first, a suitable vector harboring a polynucleotide encoding the objective peptide in an expressible form (e.g., downstream of a regulatory sequence corresponding to a promoter sequence) is prepared and transformed into a suitable host cell. The host cell is then cultured to produce the peptide of interest. The peptide can also be produced in vitro adopting an in vitro translation system.

### IV. POLYNUCLEOTIDES

The present invention also provides a polynucleotide that encodes any of the aforementioned peptides of the present invention. These include polynucleotides derived from the natural occurring TTLL4 gene (GenBank Accession No. NM_{—}014640 (SEQ ID NO: 79)) as well as those having a conservatively modified nucleotide sequence thereof. Herein, the phrase “conservatively modified nucleotide sequence” refers to sequences which encode identical or essentially identical amino acid sequences. Due to the degeneracy of the genetic code, a large number of functionally identical nucleic acids encode any given protein. For instance, the codons GCA, GCC, GCG, and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a peptide also describes every possible silent variation of the nucleic acid. One of ordinary skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a peptide is implicitly described in each disclosed sequence.

The polynucleotide of the present invention can be composed of DNA, RNA, and derivatives thereof. As is well known in the art, a DNA is suitably composed of bases such as A, T, C, and G, and T is replaced by U in an RNA. One of skill will recognize that non-naturally occurring bases may be included in polynucleotides, as well.

The polynucleotide of the present invention can encode multiple peptides of the present invention with or without intervening amino acid sequences in between. For example, the intervening amino acid sequence can provide a cleavage site (e.g., enzyme recognition sequence) of the polynucleotide or the translated peptides. Furthermore, the polynucleotide can include any additional sequences to the coding sequence encoding the peptide of the present invention. For example, the polynucleotide can be a recombinant polynucleotide that includes regulatory sequences required for the expression of the peptide or can be an expression vector (plasmid) with marker genes and such. In general, such recombinant polynucleotides can be prepared by the manipulation of polynucleotides through conventional recombinant techniques using, for example, polymerases and endonucleases.

Both recombinant and chemical synthesis techniques can be used to produce the polynucleotides of the present invention. For example, a polynucleotide can be produced by insertion into an appropriate vector, which can be expressed when transfected into a competent cell. Alternatively, a polynucleotide can be amplified using PCR techniques or expression in suitable hosts (see, e.g., Sambrook et al., Molecular Cloning: A Laboratory Manual, Cold Spring Harbor Laboratory, New York, 1989). Alternatively, a polynucleotide can be synthesized using the solid phase techniques, as described in Beaucage S L & Iyer R P, Tetrahedron 1992, 48: 2223-311; Matthes et al., EMBO J. 1984, 3: 801-5.

### V. EXOSOMES

The present invention further provides intracellular vesicles called exosomes, which present complexes formed between the peptides of the present invention and HLA antigens on their surface. Exosomes can be prepared, for example, using the methods detailed in Japanese Patent Application Kohyo Publications Nos. Hei 11-510507 and WO99/03499, and can be prepared using APCs obtained from patients who are subject to treatment and/or prevention. The exosomes of the present invention can be inoculated as vaccines, in a fashion similar to the peptides of the present invention.

The type of HLA antigens included in the complexes must match that of the subject requiring treatment and/or prevention. For example, in the Japanese population, HLA-A24 and HLA-A2, particularly HLA-A*2402 and HLA-A*0201 and HLA-A*0206, are prevalent and therefore would be appropriate for treatment of Japanese patients. The use of the A24 type that are highly expressed among the Japanese and Caucasian is favorable for obtaining effective results, and subtypes such as A2402, A*0201 and A*0206 also find use. Typically, in the clinic, the type of HLA antigen of the patient requiring treatment is investigated in advance, which enables the appropriate selection of peptides having high levels of binding affinity to the particular antigen, or having CTL inducibility by antigen presentation. Furthermore, in order to obtain peptides having both high binding affinity and CTL inducibility, substitution, insertion, deletion and/or addition of 1, 2, or several amino acids can be performed based on the amino acid sequence of the naturally occurring TTLL4 partial peptide.

When using the A24 type HLA antigen for the exosome of the present invention, peptides having a sequence selected from among SEQ ID NOs: 1 and 3 to 37 have particular utility.

Alternatively, when using the A2 type HLA antigen for the exosome of the present invention, peptides having a sequence selected from among SEQ ID NOs: 38 to 73 have particular utility.

In some embodiments, the exosomes of the present invention are exosomes that present a complex of the peptide of the present invention and HLA-A24 or HLA-A2 antigen on their surface.

### VI. ANTIGEN-PRESENTING CELLS (APCS)

The present invention also provides isolated antigen-presenting cells (APCs) that present complexes formed between HLA antigens and the peptides of the present invention on its surface. The APCs can be derived from patients who are subject to treatment and/or prevention, and can be administered as vaccines by themselves or in combination with other drugs including the peptides of the present invention, exosomes, or CTLs.

The APCs are not limited to a particular kind of cells and include dendritic cells (DCs), Langerhans cells, macrophages, B cells, and activated T cells, which are known to present proteinaceous antigens on their cell surface so as to be recognized by lymphocytes. Since DC is a representative APC having the strongest CTL inducing action among APCs, DCs find use as the APCs of the present invention.

For example, the APCs of the present invention can be obtained by inducing DCs from peripheral blood monocytes and then contacting (stimulating) them with the peptides of the present invention in vitro, ex vivo or in vivo. When the peptides of the present invention are administered to the subjects, APCs that present the peptides of the present invention are induced in the body of the subject. The phrase “inducing APC” includes contacting (stimulating) a cell with the peptides of the present invention, or nucleotides encoding the peptides of the present invention to present complexes formed between HLA antigens and the peptides of the present invention on cell's surface. Therefore, the APCs of the present invention can be obtained by collecting the APCs from the subject after administering the peptides of the present invention to the subject. Alternatively, the APCs of the present invention can be obtained by contacting APCs collected from a subject with the peptide of the present invention.

The APCs of the present invention can be administered to a subject for inducing immune response against cancer in the subject by themselves or in combination with other drugs including the peptides, exosomes or CTLs of the present invention. For example, the ex vivo administration can include steps of:

a: collecting APCs from a first subject,

b: contacting the APCs of step a, with the peptide, and
c: administering the APCs of step b to a second subject.
The first subject and the second subject can be the same individual, or may be different individuals.

In the context of the present invention, one may utilize the peptides of the present invention for manufacturing a pharmaceutical composition capable of inducing antigen-presenting cells. A method or process for manufacturing a pharmaceutical composition for inducing antigen-presenting cells is provided herein and preferably includes the step of admixing or formulating the peptide of the invention with a pharmaceutically acceptable carrier.

The present invention also provides for the use of the peptides of the present invention for inducing antigen-presenting cells. The APCs obtained by step b can be formulated and administered a vaccine for treating and/or preventing cancer, such as bladder cancer, cholangiocellular carcinoma, CML, colon and rectum cancer, esophageal cancer, liver cancer, lymphoma, pancreatic cancer, prostate cancer, renal carcinoma, SCLC, NSCLC, soft tissue tumor and osteosarcoma, but not limited thereto.

According to an aspect of the present invention, the APCs of the present invention have a high level of CTL inducibility. In the term of “high level of CTL inducibility”, the high level is relative to the level of that by APC contacted with no peptide or peptides which can not induce the CTL. Such APCs having a high level of CTL inducibility can be prepared by a method that includes the step of transferring a polynucleotide encoding the peptide of the present invention to APCs in vitro as well as the method mentioned above. The introduced genes can be in the form of DNAs or RNAs. Examples of methods for introduction include, without particular limitations, various methods conventionally performed in this field, such as lipofection, electroporation, and calcium phosphate method can be used. More specifically, it can be performed as described in Cancer Res 1996, 56: 5672-7; J Immunol 1998, 161: 5607-13; J Exp Med 1996, 184: 465-72; Published Japanese Translation of International Publication No. 2000-509281. By transferring the gene into APCs, the gene undergoes transcription, translation, and such in the cell, and then the obtained protein is processed by MHC Class I or Class II, and proceeds through a presentation pathway to present partial peptides.

In some embodiments, the APCs of the present invention are APCs that present complexes of HLA-A24 or HLA-A2 antigen and the peptide of the present invention on their surface.

### VII. CYTOTOXIC T LYMPHOCYTES (CTLS)

A CTL induced against any one of the peptides of the present invention strengthens the immune response targeting cancer cells in vivo and thus can be used as vaccines, in a fashion similar to the peptides per se. Thus, the present invention provides isolated CTLs that are specifically induced or activated by any one of the present peptides.

Such CTLs can be obtained by (1) administering the peptide(s) of the present invention to a subject or (2) contacting (stimulating) subject-derived APCs, and CD8 positive cells, or peripheral blood mononuclear leukocytes in vitro with the peptide(s) of the present invention or (3) contacting CD8 positive T cells or peripheral blood mononuclear leukocytes in vitro with the APCs or exosomes presenting a complex of an HLA antigen and the peptide on its surface or (4) introducing a gene that includes a polynucleotide encoding a T cell receptor (TCR) subunit biding to the peptide of the present invention. Such APCs or exosomes can be prepared by the methods described above and details of the method of (4) is described bellow in section “VIII. T cell receptor (TCR)”.

The CTLs of the present invention can be derived from patients who are subject to treatment and/or prevention, and can be administered by themselves or in combination with other drugs including the peptides of the present invention or exosomes for the purpose of regulating effects. The obtained CTLs act specifically against target cells presenting the peptides of the present invention, for example, the same peptides used for induction. The target cells can be cells that endogenously express TTLL4, such as cancer cells, or cells that are transfected with the TTLL4 gene; and cells that present a peptide of the present invention on the cell surface due to stimulation by the peptide can also serve as targets of activated CTL attack.

In some embodiments, the CTLs of the present invention are CTLs that recognize cells presenting complexes of HLA-A24 or HLA-A2 antigen and the peptide of the present invention. In the context of the CTL, the phrase “recognize a cell” refers to binding a complex of HLA-A24 or HLA-A2 antigen and the peptide of the present invention on the cell surface via its TCR and showing specific cytotoxic activity against the cell. Herein, “specific cytotoxic activity” refers to showing cytotoxic activity against the cell presenting a complex of HLA-A24 or HLA-A2 antigen and the peptide of the present invention but not other cells.

### VIII. T CELL RECEPTOR (TCR)

The present invention also provides a composition including nucleic acids encoding polypeptides that are capable of forming a subunit of a T cell receptor (TCR), and methods of using the same. The TCR subunits have the ability to form TCRs that confer specificity to T cells against tumor cells presenting TTLL4. By using the known methods in the art, the nucleic acids of alpha- and beta-chains as the TCR subunits of the CTL induced with one or more peptides of the present invention can be identified (WO2007/032255 and Morgan et al., J Immunol, 171, 3288 (2003)). For example, the PCR method is preferred to analyze the TCR. The PCR primers for the analysis can be, for example, 5′-R primers (5′-gtctaccaggcattcgcttcat-3′) as 5′ side primers (SEQ ID NO: 81) and 3-TRa-C primers (5′-tcagctggaccacagccgcagcgt-3′) specific to TCR alpha chain C region (SEQ ID NO: 82), 3-TRb-C1 primers (5′-tcagaaatcctttctcttgac-3′) specific to TCR beta chain C1 region (SEQ ID NO: 83) or 3-TRbeta-C2 primers (5′-ctagcctctggaatcctttctctt-3′) specific to TCR beta chain C2 region (SEQ ID NO: 84) as 3′ side primers, but not limited thereto. The derivative TCRs can bind target cells displaying the TTLL4 peptide with high avidity, and optionally mediate efficient killing of target cells presenting the TTLL4 peptide in vivo and in vitro.

The nucleic acids encoding the TCR subunits can be incorporated into suitable vectors, e.g., retroviral vectors. These vectors are well known in the art. The nucleic acids or the vectors including them usefully can be transferred into a T cell, for example, a T cell from a patient. Advantageously, the invention provides an off-the-shelf composition allowing rapid modification of a patient's own T cells (or those of another mammal) to rapidly and easily produce modified T cells having excellent cancer cell killing properties.

The specific TCR is a receptor capable of specifically recognizing a complex of a peptide of the present invention and HLA molecule, giving a T cell specific activity against the target cell when the TCR is presented on the surface of the T cell. A specific recognition of the above complex may be confirmed by any known methods, preferred examples of which include HLA multimer staining analysis using HLA molecules and peptides of the present invention, and ELISPOT assay. By performing the ELISPOT assay, it can be confirmed that a T cell expressing the TCR on the cell surface recognizes a cell by the TCR, and that the signal is transmitted intracellularly. The confirmation that the above-mentioned complex can give a T cell cytotoxic activity when the complex exists on the T cell surface may also be carried out by a known method. A preferred method includes, for example, the determination of cytotoxic activity against an HLA positive target cell, such as chromium release assay.

Also, the present invention provides CTLs which are prepared by transduction with the nucleic acids encoding the TCR subunits polypeptides that bind to the TTLL4 peptide, e.g., SEQ ID NOs: 1 and 3 to 37 in the context of HLA-A24, and also the peptides of SEQ ID NOs: 38 to 73 in the context of HLA-A2.

The transduced CTLs are capable of homing to cancer cells in vivo, and can be expanded by well known culturing methods in vitro (e.g., Kawakami et al., J. Immunol., 142, 3452-3461 (1989)). The CTLs of the present invention can be used to form an immunogenic composition useful in treating or the prevention of cancer in a patient in need of therapy or protection (See WO2006/031221 the contents of which are incorporated by reference herein).

### IX. PHARMACEUTICAL COMPOSITIONS

Since TTLL4 expression is specifically elevated in cancers, examples of which include, but are not limited to, bladder cancer, cholangiocellular carcinoma, CML, colon and rectum cancer, esophageal cancer, liver cancer, lymphoma, pancreatic cancer, prostate cancer, renal carcinoma, SCLC, NSCLC, soft tissue tumor and osteosarcoma, the peptides of or polynucleotides of the present invention may be used for treating and/or for the prophylaxis of cancer, and/or for the prevention of a post-operative recurrence thereof. Thus, the present invention provides a pharmaceutical composition or agents formulated for the treatment and/or prophylaxis cancer, and/or for the prevention of a postoperative recurrence thereof, such composition or agent including as active ingredient one or more of the peptides, or polynucleotides of the present invention as an active ingredient. Alternatively, the present peptides can be expressed on the surface of any of the foregoing exosomes or cells, such as APCs for the use as pharmaceutical compositions. In addition, the aforementioned CTLs which target any one of the peptides of the present invention can also be used as the active ingredient of the present pharmaceutical compositions.

Accordingly, the present invention provide agents or compositions including at least one active ingredient selected from among:

(a) one or more peptides of the present invention;

(b) one or more polynucleotides encoding such a peptide as disclosed herein in an expressible form;

(c) one or more APCs or an exosomes of the present invention; and

(d) one or more CTLs of the present invention.

The pharmaceutical compositions of the present invention also find use as a vaccine. In the context of the present invention, the phrase “vaccine” (also referred to as an “immunogenic composition”) refers to an agent or composition that has the function to improve, enhance and/or induce anti-tumor immunity upon inoculation into animals. In other words, the present invention provides the pharmaceutical agents or compositions of the present invention for inducing an immune response against cancer in a subject.

The pharmaceutical compositions of the present invention can be used to treat and/or prevent cancers, and/or prevention of a postoperative recurrence thereof in subjects or patients including human and any other mammal including, but not limited to, mouse, rat, guinea-pig, rabbit, cat, dog, sheep, goat, pig, cattle, horse, monkey, baboon, and chimpanzee, particularly a commercially important animal or a domesticated animal. In some embodiments, the pharmaceutical agents or compositions of the present invention can be formulated for the administration to a subject whose HLA antigen is HLA-A24 or HLA-A2.

In another embodiment, the present invention also provides the use of an active ingredient in manufacturing a pharmaceutical composition or agent for treating cancer or tumor, said active ingredient selected from among:

(a) a peptide of the present invention;

(b) a polynucleotide encoding such a peptide as disclosed herein in an expressible form;

(c) an APC or an exosome presenting a peptide of the present invention on its surface; and

(d) a cytotoxic T cell of the present invention.

Alternatively, the present invention further provides an active ingredient for use in the treatment and/or prevention of cancers or tumors, said active ingredient selected from among:

(a) a peptide of the present invention;

(b) a polynucleotide encoding such a peptide as disclosed herein in an expressible form;

(c) an APC or an exosome presenting a peptide of the present invention on its surface; and

(d) a cytotoxic T cell of the present invention.

Alternatively, the present invention further provides a method or process for manufacturing a pharmaceutical composition or substance for treating or preventing cancer or tumor, wherein the method or process includes the step of formulating a pharmaceutically or physiologically acceptable carrier with an active ingredient selected from among:

(a) a peptide of the present invention;

(b) a polynucleotide encoding such a peptide as disclosed herein in an expressible form;

(c) an APC or an exosome presenting a peptide of the present invention on its surface; and

(d) a cytotoxic T cell of the present invention.

In another embodiment, the present invention also provides a method or process for manufacturing a pharmaceutical composition or agent for treating or preventing cancer or tumor, wherein the method or process includes the steps of admixing an active ingredient with a pharmaceutically or physiologically acceptable carrier, wherein the active ingredient is selected from among:

(a) a peptide of the present invention;

(b) a polynucleotide encoding such a peptide as disclosed herein in an expressible form;

(c) an APC or an exosome presenting a peptide of the present invention on its surface; and

(d) a cytotoxic T cell of the present invention.

According to the present invention, peptides having an amino acid sequence selected from among SEQ ID NOs: 1 and 3 to 37 have been found to be HLA-A24 restricted epitope peptides or the candidates and also SEQ ID NOs: 38 to 73 have been found to be HLA-A2 restricted epitope peptides or the candidates that can induce potent and specific immune response against cancer expressing HLA-A24 or HLA-A2 and TTLL4 in a subject. Therefore, the present pharmaceutical compositions or agents which include any of these peptides with the amino acid sequences of SEQ ID NOs: 1, 3 to 37 and 38 to 73 are particularly suited for the administration to subjects whose HLA antigen is HLA-A24 and HLA-A2 respectively. The same applies to pharmaceutical compositions or agents that contain polynucleotides encoding any of these peptides (i.e., the polynucleotides of the present invention).

Cancers to be treated by the pharmaceutical compositions or agents of the present invention are not limited and include all kinds of cancers wherein TTLL4 is involved, including, but not limited to, bladder cancer, cholangiocellular carcinoma, CML, colon and rectum cancer, esophageal cancer, liver cancer, lymphoma, pancreatic cancer, prostate cancer, renal carcinoma, SCLC, NSCLC, soft tissue tumor and osteosarcoma.

The pharmaceutical compositions or agents of the present invention can contain in addition to the aforementioned active ingredients, other peptides which have the ability to induce CTLs against cancerous cells, other polynucleotides encoding the other peptides, other cells that present the other peptides, or such. Herein, the other peptides that have the ability to induce CTLs against cancerous cells are exemplified by cancer specific antigens (e.g., identified TAAs), but are not limited thereto.

If needed, the pharmaceutical compositions or agents of the present invention can optionally include other therapeutic substances as an active ingredient, so long as the substance does not inhibit the antitumoral effect of the active ingredient, e.g., any of the present peptides. For example, formulations can include anti-inflammatory compositions, pain killers, chemotherapeutics, and the like. In addition to including other therapeutic substances in the medicament itself, the medicaments of the present invention can also be administered sequentially or concurrently with the one or more other pharmacologic compositions. The amounts of medicament and pharmacologic composition depend, for example, on what type of pharmacologic composition(s) is/are used, the disease being treated, and the scheduling and routes of administration.

It should be understood that in addition to the ingredients particularly mentioned herein, the pharmaceutical compositions or agent of the present invention can include other compositions conventional in the art having regard to the type of formulation in question.

In one embodiment of the present invention, the pharmaceutical compositions or agents of the present invention can be included in articles of manufacture and kits containing materials useful for treating the pathological conditions of the disease to be treated, e.g., cancer. The article of manufacture can include a container of any of the present pharmaceutical compositions or agents with a label. Suitable containers include bottles, vials, and test tubes. The containers can be formed from a variety of materials, such as glass or plastic. The label on the container should indicate the composition or agent is used for treating or prevention of one or more conditions of the disease. The label can also indicate directions for administration and so on.

In addition to the container described above, a kit including a pharmaceutical composition or agent of the present invention can optionally further include a second container housing a pharmaceutically-acceptable diluent. It can further include other materials desirable from a commercial and user standpoint, including other buffers, diluents, filters, needles, syringes, and package inserts with instructions for use.

The pharmaceutical compositions or agents can, if desired, be presented in a pack or dispenser device which can contain one or more unit dosage forms containing the active ingredient. The pack can, for example, include metal or plastic foil, such as a blister pack. The pack or dispenser device can be accompanied by instructions for administration.

(1) Pharmaceutical Compositions Containing the Peptides as the Active Ingredient

The peptides of this invention can be administered directly as a pharmaceutical composition or agent, or if necessary may be formulated by conventional formulation methods. In the latter case, in addition to the peptides of this invention, carriers, excipients, and such that are ordinarily used for drugs can be included as appropriate without particular limitations. Examples of such carriers are sterilized water, physiological saline, phosphate buffer, culture fluid and such. Furthermore, the pharmaceutical compositions or agents can contain as necessary, stabilizers, suspensions, preservatives, surfactants and such. The pharmaceutical compositions or agents of the present invention can be used for anticancer purposes.

The peptides of the present invention can be prepared as a combination composed of two or more of peptides of the present invention, to induce CTL in vivo. The peptide combination can take the form of a cocktail or can be conjugated to each other using standard techniques. For example, the peptides can be chemically linked or expressed as a single fusion polypeptide sequence. The peptides in the combination can be the same or different. By administering the peptides of the present invention, the peptides are presented at a high density by the HLA antigens on APCs, then CTLs that specifically react toward the complex formed between the displayed peptide and the HLA antigen are induced. Alternatively, APCs (e.g., DCs) are removed from subjects and then stimulated by the peptides of the present invention to obtain APCs that present any of the peptides of the present invention on their cell surface. These APCs are readministered to the subjects to induce CTLs in the subjects, and as a result, aggressiveness towards the tumor-associated endothelium can be increased.

The pharmaceutical compositions or agents for the treatment and/or prevention of cancer containing any peptide of the present invention as the active ingredient can also include an adjuvant known to effectively establish cellular immunity. Alternatively, the pharmaceutical compositions or agents can be administered with other active ingredients, or administered by formulation into granules. An adjuvant refers to a compound that enhances the immune response against the protein when administered together (or successively) with the protein having immunological activity. Adjuvants contemplated herein include those described in the literature (Clin Microbiol Rev 1994, 7: 277-89). Examples of suitable adjuvants include, but are not limited to, aluminum phosphate, aluminum hydroxide, alum, cholera toxin, salmonella toxin, IFA (Incomplete Freund's adjuvant), CFA (Complete Freund's adjuvant) and the like.

Furthermore, liposome formulations, granular formulations in which the peptide is bound to few-micrometers diameter beads, and formulations in which a lipid is bound to the peptide may be conveniently used.

In another embodiment of the present invention, the peptides of the present invention may also be administered in the form of a pharmaceutically acceptable salt. Examples of preferred salts include salts with an alkali metal, salts with a metal, salts with an organic base, salts with an organic acid and salts with an inorganic acid. As used herein, “pharmaceutically acceptable salt” refers to those salts which retain the biological effectiveness and properties of the compound and which are obtained by reaction with inorganic acids or bases such as hydrochloric acid, hydrobromic acid, sulfuric acid, nitric acid, phosphoric acid, methanesulfonic acid, ethanesulfonic acid, p-toluenesulfonic acid, salicylic acid and the like.

In some embodiments, the pharmaceutical compositions or agents of the present invention may further include a component that primes CTL. Lipids have been identified as compositions capable of priming CTL in vivo against viral antigens. For example, palmitic acid residues can be attached to the epsilon- and alpha-amino groups of a lysine residue and then linked to a peptide of the invention. The lipidated peptide can then be administered either directly in a micelle or particle, incorporated into a liposome, or emulsified in an adjuvant. As another example of lipid priming of CTL responses, E. coli lipoproteins, such as tripalmitoyl-S-glycerylcysteinyl-seryl-serine (P3CSS) can be used to prime CTL when covalently attached to an appropriate peptide (see, e.g., Deres et al., Nature 1989, 342: 561-4).

The method of administration can be oral, intradermal, subcutaneous, intravenous injection, or such, and systemic administration or local administration to the vicinity of the targeted sites. The administration can be performed by single administration or boosted by multiple administrations. The dose of the peptides of the present invention can be adjusted appropriately according to the disease to be treated, age of the patient, weight, method of administration, and such, and is ordinarily 0.001 mg to 1000 mg, for example, 0.01 mg to 100 mg, for example, 0.1 mg to 10 mg, and can be administered once in a few days to few months. One skilled in the art can appropriately select a suitable dose.

(2) Pharmaceutical Compositions Containing Polynucleotides as the Active Ingredient

The pharmaceutical compositions or agents of the invention can also contain nucleic acids encoding the peptides disclosed herein in an expressible form. Herein, the phrase “in an expressible form” means that the polynucleotide, when introduced into a cell, will be expressed in vivo as a polypeptide that induces anti-tumor immunity. In an exemplified embodiment, the nucleic acid sequence of the polynucleotide of interest includes regulatory elements necessary for expression of the polynucleotide. The polynucleotide(s) can be equipped so to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivacaine, polymers, peptide-mediated) delivery, cationic lipid complexes, and particle-mediated (“gene gun”) or pressure-mediated delivery (see, e.g., U.S. Pat. No. 5,922,687).

The peptides of the present invention can also be expressed by viral or bacterial vectors. Examples of expression vectors include attenuated viral hosts, such as vaccinia or fowlpox. This approach involves the use of vaccinia virus, e.g., as a vector to express nucleotide sequences that encode the peptide. Upon introduction into a host, the recombinant vaccinia virus expresses the immunogenic peptide, and thereby elicits an immune response. Vaccinia vectors and methods useful in immunization protocols are described in, e.g., U.S. Pat. No. 4,722,848. Another vector is BCG (Bacille Calmette Guerin). BCG vectors are described in Stover et al., Nature 1991, 351: 456-60. A wide variety of other vectors useful for therapeutic administration or immunization e.g., adeno and adeno-associated virus vectors, retroviral vectors, Salmonella typhi vectors, detoxified anthrax toxin vectors, and the like, will be apparent. See, e.g., Shata et al., Mol Med Today 2000, 6: 66-71; Shedlock et al., J Leukoc Biol 2000, 68: 793-806; Hipp et al., In Vivo 2000, 14: 571-85.

Delivery of a polynucleotide into a patient can be either direct, in which case the patient is directly exposed to a polynucleotide-carrying vector, or indirect, in which case, cells are first transformed with the polynucleotide of interest in vitro, then the cells are transplanted into the patient. Theses two approaches are known, respectively, as in vivo and ex vivo gene therapies.

For general reviews of the methods of gene therapy, see Goldspiel et al., Clinical Pharmacy 1993, 12: 488-505; Wu and Wu, Biotherapy 1991, 3: 87-95; Tolstoshev, Ann Rev Pharmacol Toxicol 1993, 33: 573-96; Mulligan, Science 1993, 260: 926-32; Morgan & Anderson, Ann Rev Biochem 1993, 62: 191-217; Trends in Biotechnology 1993, 11(5): 155-215). Methods commonly known in the art of recombinant DNA technology that are applicable to the present invention are described by Ausubel et al. in Current Protocols in Molecular Biology (John Wiley & Sons, NY, 1993); and Krieger in Gene Transfer and Expression, A Laboratory Manual (Stockton Press, NY, 1990).

The method of administration can be oral, intradermal, subcutaneous, intravenous injection, or such, and systemic administration or local administration to the vicinity of the targeted sites finds use. The administration can be performed by single administration or boosted by multiple administrations. The dose of the polynucleotide in the suitable carrier or cells transformed with the polynucleotide encoding the peptides of the present invention can be adjusted appropriately according to the disease to be treated, age of the patient, weight, method of administration, and such, and is ordinarily 0.001 mg to 1000 mg, for example, 0.01 mg to 100 mg, for example, 0.1 mg to 10 mg, and can be administered once every a few days to once every few months. One skilled in the art can appropriately select the suitable dose.

### X. METHODS USING THE PEPTIDES, EXOSOMES, APCS AND CTLS

The peptides and polynucleotides of the present invention can be used for inducing APCs and CTLs. The exosomes and APCs of the present invention can be also used for inducing CTLs. The peptides, polynucleotides, exosomes and APCs can be used in combination with any other compounds so long as the additional compounds do not inhibit CTL inducibility. Thus, any of the aforementioned pharmaceutical compositions or agents of the present invention can be used for inducing CTLs. In addition thereto, those including the peptides and polynucleotides can be also used for inducing APCs as discussed below.

(1) Method of Inducing Antigen-Presenting Cells (APCs)

The present invention provides methods of inducing APCs with high CTL inducibility using the peptides or polynucleotides of the present invention.

The methods of the present invention include the step of contacting APCs with the peptides of the present invention in vitro, ex vivo or in vivo. For example, the method contacting APCs with the peptides ex vivo can include steps of:

a: collecting APCs from a subject, and

b: contacting the APCs of step a with the peptide.

The APCs are not limited to a particular kind of cells and include DCs, Langerhans cells, macrophages, B cells, and activated T cells, which are known to present proteinaceous antigens on their cell surface so as to be recognized by lymphocytes. Preferably, DCs can be used since they have the strongest CTL inducibility among APCs. Any one of peptide of the present invention can be used by itself or in combination with other peptides of the present invention or CTL inducible peptides derived from TAA other than TTLL4.

On the other hand, when the peptides of the present invention are administered to a subject, the APCs are contacted with the peptides in vivo, and consequently, the APCs with high CTL inducibility are induced in the body of the subject. Thus, the method of the present invention includes administering the peptides of the present invention to a subject to induce APCs with CTL inducibility in the body of the subject. Similarly, when the polynucleotides of this invention are administered to a subject in an expressible form, the peptides of the present invention are expressed and contacted with APCs in vivo, and consequently, the APCs with high CTL inducibility are induced in the body of the subject. Thus, the present invention also includes administering the polynucleotides of the present invention to a subject to induce APCs with CTL inducibility in the body of the subject. The phrase “expressible form” was described above in section “IX. Pharmaceutical compositions (2) Pharmaceutical compositions containing polynucleotides as the active ingredient”.

The present invention also includes introducing the polynucleotide of the present invention into an APCs to induce APCs with CTL inducibility. For example, the method can include steps of:

a: collecting APCs from a subject, and

b: introducing a polynucleotide encoding peptide of the present invention.

Step b can be performed as described above in section “VI. Antigen-presenting cells”.

Alternatively, the present invention provides a method for preparing an antigen-presenting cell (APC) which specifically induces CTL activity against TTLL4, wherein the method can include one of the following steps:

(a) contacting an APC with a peptide of the present invention in vitro, ex vivo or in vivo; and

(b) introducing a polynucleotide encoding a peptide of the present invention into an APC.

Alternatively, the present invention provides methods for inducing an APC having CTL inducibility, wherein the methods include the step selected from among:

(a) contacting an APC with the peptide of the present invention; and

(b) introducing the polynucleotide encoding the peptide of the present invention into an APC.

The methods of the present invention can be carried out in vitro, ex vivo or in vivo. Preferably, the methods of the present invention can be carried out in vitro or ex vivo. APCs used for induction of APCs having CTL inducibility can be preferably APCs expressing HLA-A24 or HLA-A2 antigen. Such APCs can be prepared by the methods well-known in the arts from peripheral blood mononuclear cells (PBMCs) obtained from a subject whose HLA antigen is HLA-A24 or HLA-A2. The APCs induced by the method of the present invention can be APCs that present a complex of the peptide of the present invention and HLA antigen (HLA A24 or HLA-A2 antigen) in its surface. When APCs induced by the method of the present invention are administered to a subject in order to induce immune responses against cancer in the subject, the subject is preferably the same one from whom APCs are derived. However, the subject may be a different one from the APC donor so long as the subject has the same HLA type with the APC donor.

In another embodiment, the present invention provide agents or compositions for use in inducing an APC having CTL inducibility, and such agents or compositions include one or more peptides or polynucleotides of the present invention.

In another embodiment, the present invention provides the use of the peptide of the present invention or the polynucleotide encoding the peptide in the manufacture of an agent or composition formulated for inducing APCs.

Alternatively, the present invention further provides the peptide of the present invention or the polypeptide encoding the peptide for use in inducing an APC having CTL inducibility.

(2) Method of Inducing CTLs

The present invention also provides methods for inducing CTLs using the peptides, polynucleotides, or exosomes or APCs of the present invention.

The present invention also provides methods for inducing CTLs using a polynucleotide encoding a polypeptide that is capable of forming a T cell receptor (TCR) subunit recognizing a complex of the peptides of the present invention and HLA antigens. Preferably, the methods for inducing CTLs include at least one step selected from among:

a: contacting a CD8 positive T cell with an antigen-presenting cell and/or an exosome that presents on its surface a complex of an HLA antigen and a peptide of the preset invention; and

b: introducing a polynucleotide encoding a polypeptide that is capable of forming a TCR subunit recognizing a complex of a peptide of the present invention and an HLA antigen into a CD8 positive T cell.

When the peptides, the polynucleotides, APCs, or exosomes of the present invention are administered to a subject, CTL is induced in the body of the subject, and the strength of the immune response targeting the cancer cells expressing TTLL4 is enhanced. Thus, the methods of the present invention include the step of administering the peptides, the polynucleotides, the APCs or exosomes of the present invention to a subject.

Alternatively, CTL can be also induced by using them ex vivo or in vivo, and after inducing CTL, the activated CTLs are returned to the subject. For example, the method can include steps of:

a: collecting APCs from subject,

b: contacting the APCs of step a, with the peptide, and

c: co-culturing the APCs of step b with CD8 positive T cells.

The APCs to be co-cultured with the CD8 positive T cells in above step c can also be prepared by transferring a gene that includes a polynucleotide of the present invention into APCs as described above in section “VI. Antigen-presenting cells”, although the present invention is not limited thereto and thus encompasses any APCs that effectively present on its surface a complex of an HLA antigen and a peptide of the present invention.

Instead of such APCs, the exosomes that presents on its surface a complex of an HLA antigen and the peptide of the present invention can be also used. Namely, the present invention can includes the step of co-culturing exosomes presenting on its surface a complex of an HLA antigen and the peptide of the present invention. Such exosomes can be prepared by the methods described above in section “V. Exosomes”.

Furthermore, CTL can be induced by introducing a gene that includes a polynucleotide encoding the TCR subunit binding to the peptide of the present invention into CD8 positive T cells. Such transduction can be performed as described above in section “VIII. T cell receptor (TCR)”.

The methods of the present invention can be carried out in vitro, ex vivo or in vivo. Preferably, the methods of the present invention can be carried out in vitro or ex vivo. CD8 positive T cells used for induction of CTLs can be prepared by well-known methods in the art from PBMCs obtained from a subject. In preferred embodiments, the donor for CD8 positive T cells can be a subject whose HLA antigen is HLA-A24 or HLA-A2. The CTLs induced by the methods of the present invention can be CTLs that can recognize cells presenting a complex of the peptide of the present invention and HLA antigen on its surface. When CTLs induced by the method of the present invention are administered to a subject in order to induce immune responses against cancer in the subject, the subject is preferably the same one from whom CD8 positive T cells are derived. However, the subject may be a different one from the CD8 positive T cell donor so long as the subject has the same HLA type with the CD8 positive T cell donor.

In addition, the present invention provides a method or process for manufacturing a pharmaceutical composition or agent inducing CTLs, wherein the method includes the step of admixing or formulating the peptide of the present invention with a pharmaceutically acceptable carrier.

In another embodiment, the present invention provide an agent or composition for inducing CTL, wherein the agent or composition comprises one or more peptide(s), one or more polynucleotide(s), or one o more APCs or exosomes of the present invention.

In another embodiment, the present invention provides the use of the peptide, the polynucleotide, or APC or exosome of the present invention in the manufacture of an agent or composition formulated for inducing a CTL.

Alternatively, the present invention further provides the peptide, the polynucleotide, or APC or exosome of the present invention for use in inducing a CTL.

### XI. METHODS OF INDUCING IMMUNE RESPONSE

Moreover, the present invention provides methods of inducing immune response against diseases related to TTLL4. Suitable diseases include cancer, examples of which include, but are not limited to, bladder cancer, cholangiocellular carcinoma, CML, colon and rectum cancer, esophageal cancer, liver cancer, lymphoma, pancreatic cancer, prostate cancer, renal carcinoma, SCLC, NSCLC, soft tissue tumor and osteosarcoma.

The methods of the present invention may include the step of administering substance(s) or composition(s) containing any of the peptides of the present invention or polynucleotides encoding them. The inventive methods also contemplate the administration of exosomes or APCs presenting any of the peptides of the present invention. For details, see the item of “IX. Pharmaceutical compositions”, particularly the part describing the use of the pharmaceutical compositions of the present invention as vaccines. In addition, the exosomes and APCs that can be employed for the present methods for inducing immune response are described in detail under the items of “V. Exosomes”, “VI. Antigen-presenting cells (APCs)”, and (1) and (2) of “X. Methods using the peptides, exosomes, APCs and CTLs”, supra.

The present invention also provides a method or process for manufacturing a pharmaceutical composition or substance inducing immune response, wherein the method may include the step of admixing or formulating the peptide of the present invention with a pharmaceutically acceptable carrier.

Alternatively, the method of the present invention may include the step of administrating a vaccine or a pharmaceutical composition or substance of the present invention that contains:

(a) a peptide of the present invention;
(b) a nucleic acid encoding such a peptide as disclosed herein in an expressible form;
(c) an APC or an exosome presenting a peptide of the present invention on its surface; or
(d) a cytotoxic T cell of the present invention.

In the context of the present invention, a cancer over-expressing TTLL4 can be treated with these active ingredients. Examples of such cancer include, but are not limited to, bladder cancer, cholangiocellular carcinoma, CML, colon and rectum cancer, esophageal cancer, liver cancer, lymphoma, pancreatic cancer, prostate cancer, renal carcinoma, SCLC, NSCLC, soft tissue tumor and osteosarcoma. Accordingly, prior to the administration of the vaccines or pharmaceutical compositions or substance including the active ingredients, it is preferable to confirm whether the expression level of TTLL4 in the subject to be treated is enhanced. Thus, in one embodiment, the present invention provides a method for treating cancer (over)expressing TTLL4 in a patient in need thereof, such method including the steps of:

i) determining the expression level of TTLL4 in biological sample(s) obtained from a subject with the cancer to be treated;

ii) comparing the expression level of TTLL4 with normal control; and

iii) administrating at least one component selected from among (a) to (d) described above to a subject with cancer over-expressing TTLL4 compared with normal control.

Alternatively, the present invention provides a vaccine or pharmaceutical composition including at least one component selected from among (a) to (d) described above, to be administered to a subject having cancer over-expressing TTLL4. In other words, the present invention further provides a method for identifying a subject to be treated with the TTLL4 polypeptide of the present invention, such method including the step of determining an expression level of TTLL4 in subject-derived biological sample(s), wherein an increase of the level compared to a normal control level of the gene indicates that the subject may have cancer which may be treated with the TTLL4 polypeptide of the present invention. The methods of treating cancer of the present invention will be described in more detail in below.

Any subject-derived cell or tissue can be used for the determination of TTLL4 expression so long as it includes the objective transcription or translation product of TTLL4. Examples of suitable samples include, but are not limited to, bodily tissues and fluids, such as blood, sputum and urine. Preferably, the subject-derived cell or tissue sample contains a cell population including an epithelial cell, more preferably a cancerous epithelial cell or an epithelial cell derived from tissue suspected to be cancerous. Further, if necessary, the cell may be purified from the obtained bodily tissues and fluids, and then used as the subjected-derived sample.

A subject to be treated by the present method is preferably a mammal. Exemplary mammals include, but are not limited to, e.g., human, non-human primate, mouse, rat, dog, cat, horse, and cow.

According to the present invention, the expression level of TTLL4 in biological sample obtained from a subject may be determined. The expression level can be determined at the transcription (nucleic acid) product level, using methods known in the art. For example, the mRNA of TTLL4 may be quantified using probes by hybridization methods (e.g., Northern hybridization). The detection may be carried out on a chip or an array. The use of an array is preferable for detecting the expression level of TTLL4. Those skilled in the art can prepare such probes utilizing the sequence information of TTLL4. For example, the cDNA of TTLL4 may be used as the probes. If necessary, the probes may be labeled with a suitable label, such as dyes, fluorescent substances and isotopes, and the expression level of the gene may be detected as the intensity of the hybridized labels.

Furthermore, the transcription product of TTLL4 may be quantified using primers by amplification-based detection methods (e.g., RT-PCR). Such primers may be prepared based on the available sequence information of the gene.

Specifically, a probe or primer used for the present method hybridizes under stringent, moderately stringent, or low stringent conditions to the mRNA of TTLL4. As used herein, the phrase “stringent (hybridization) conditions” refers to conditions under which a probe or primer will hybridize to its target sequence, but not to other sequences. Stringent conditions are sequence-dependent and will be different under different circumstances. Specific hybridization of longer sequences is observed at higher temperatures than shorter sequences. Generally, the temperature of a stringent condition is selected to be about 5 degree Centigrade lower than the thermal melting point (Tm) for a specific sequence at a defined ionic strength and pH. The Tm is the temperature (under a defined ionic strength, pH and nucleic acid concentration) at which 50% of the probes complementary to their target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degree Centigrade for short probes or primers (e.g., 10 to 50 nucleotides) and at least about 60 degree Centigrade for longer probes or primers. Stringent conditions may also be achieved with the addition of destabilizing substances, such as formamide.

A probe or primer of the present invention is typically a substantially purified oligonucleotide. The oligonucleotide typically includes a region of nucleotide sequence that hybridizes under stringent conditions to at least about 2000, 1000, 500, 400, 350, 300, 250, 200, 150, 100, 50, or 25, consecutive sense strand nucleotide sequence of a nucleic acid including a TTTL4 sequence, or an anti-sense strand nucleotide sequence of a nucleic acid including a TTTL4 sequence, or of a naturally occurring mutant of these sequences. In particular, for example, in a preferred embodiment, an oligonucleotide having 5-50 in length can be used as a primer for amplifying the genes, to be detected. More preferably, mRNA or cDNA of a TTTL4 gene can be detected with oligonucleotide probe or primer of a specific size, generally 15-30 b in length. The size may range from at least 10 nucleotides, at least 12 nucleotides, at least 15 nucleotides, at least 20 nucleotides, at least 25 nucleotides, at least 30 nucleotides and the probes and primers may range in size from 5-10 nucleotides, 10-15 nucleotides, 15-20 nucleotides, 20-25 nucleotides and 25-30 nucleotides. In preferred embodiments, length of the oligonucleotide probe or primer can be selected from 15-25. Assay procedures, devices, or reagents for the detection of gene by using such oligonucleotide probe or primer are well known (e.g. oligonucleotide microarray or PCR). In these assays, probes or primers can also include tag or linker sequences. Further, probes or primers can be modified with detectable label or affinity ligand to be captured. Alternatively, in hybridization based detection procedures, a polynucleotide having a few hundreds (e.g., about 100-200) bases to a few kilo (e.g., about 1000-2000) bases in length can also be used for a probe (e.g., northern blotting assay or cDNA microarray analysis).

Alternatively, the translation product may be detected for the diagnosis of the present invention. For example, the quantity of TTLL4 protein (SEQ ID NO: 80) or the immunologically fragment thereof may be determined. Methods for determining the quantity of the protein as the translation product include immunoassay methods that use an antibody specifically recognizing the protein. The antibody may be monoclonal or polyclonal. Furthermore, any fragment or modification (e.g., chimeric antibody, scFv, Fab, F(ab′)₂, Fv, etc.) of the antibody may be used for the detection, so long as the fragment or modified antibody retains the binding ability to the TTLL4 protein. Such antibodies against the peptides of the present invention and the fragments thereof are also provided by the present invention. Methods to prepare these kinds of antibodies for the detection of proteins are well known in the art, and any method may be employed in the present invention to prepare such antibodies and equivalents thereof.

As another method to detect the expression level of TTLL4 gene based on its translation product, the intensity of staining may be measured via immunohistochemical analysis using an antibody against the TTLL4 protein. Namely, in this measurement, strong staining indicates increased presence/level of the protein and, at the same time, high expression level of TTLL4 gene.

The expression level of a target gene, e.g., the TTLL4 gene, in cancer cells can be determined to be increased if the level increases from the control level (e.g., the level in normal cells) of the target gene by, for example, 10%, 25%, or 50%; or increases to more than 1.1 fold, more than 1.5 fold, more than 2.0 fold, more than 5.0 fold, more than 10.0 fold, or more.

The control level may be determined at the same time as the cancer cells by using a sample(s) previously collected and stored from a subject/subjects whose disease state(s) (cancerous or non-cancerous) is/are known. In addition, normal cells obtained from non-cancerous regions of an organ that has the cancer to be treated may be used as normal control. Alternatively, the control level may be determined by a statistical method based on the results obtained by analyzing previously determined expression level(s) of TTLL4 gene in samples from subjects whose disease states are known. Furthermore, the control level can be derived from a database of expression patterns from previously tested cells. Moreover, according to an aspect of the present invention, the expression level of TTLL4 gene in a biological sample may be compared to multiple control levels, which are determined from multiple reference samples. It is preferred to use a control level determined from a reference sample derived from a tissue type similar to that of the subject-derived biological sample. Moreover, it is preferred to use the standard value of the expression levels of TTLL4 gene in a population with a known disease state. The standard value may be obtained by any method known in the art. For example, a range of mean+/−2 S.D. or mean+/−3 S.D. may be used as the standard value.

In the context of the present invention, a control level determined from a biological sample that is known to be non-cancerous is referred to as a “normal control level”. On the other hand, if the control level is determined from a cancerous biological sample, it is referred to as a “cancerous control level”. Difference between a sample expression level and a control level can be normalized to the expression level of control nucleic acids, e.g., housekeeping genes, whose expression levels are known not to differ depending on the cancerous or non-cancerous state of the cell. Exemplary control genes include, but are not limited to, beta-actin, glyceraldehyde 3 phosphate dehydrogenase, and ribosomal protein P1.

When the expression level of TTLL4 gene is increased as compared to the normal control level, or is similar/equivalent to the cancerous control level, the subject may be diagnosed with cancer to be treated.

The present invention also provides a method of (i) diagnosing whether a subject suspected to have cancer to be treated, and/or (ii) selecting a subject for cancer treatment, such method including the steps of:

a) determining the expression level of TTLL4 in biological sample(s) obtained from a subject who is suspected to have the cancer to be treated;

b) comparing the expression level of TTLL4 with a normal control level;

c) diagnosing the subject as having the cancer to be treated, if the expression level of TTLL4 is increased as compared to the normal control level; and

d) selecting the subject for cancer treatment, if the subject is diagnosed as having the cancer to be treated, in step c).

Alternatively, such a method may include the steps of:

a) determining the expression level of TTLL4 in biological sample(s) obtained from a subject who is suspected to have the cancer to be treated;

b) comparing the expression level of TTLL4 with a cancerous control level;

c) diagnosing the subject as having the cancer to be treated, if the expression level of TTLL4 is similar or equivalent to the cancerous control level; and

d) selecting the subject for cancer treatment, if the subject is diagnosed as having the cancer to be treated, in step c).

The present invention also provides a diagnostic kit for diagnosing or determining a subject who is or is suspected to be suffering from cancer that can be treated with the TTLL4 polypeptide of the present invention, which may also be useful in assessing and/or monitoring the efficacy or applicability of a cancer immunotherapy. Preferably, the cancer includes, but is not limited to, bladder cancer, cholangiocellular carcinoma, CML, colon and rectum cancer, esophageal cancer, liver cancer, lymphoma, pancreatic cancer, prostate cancer, renal carcinoma, SCLC, NSCLC, soft tissue tumor and osteosarcoma. More particularly, the kit preferably includes at least one reagent for detecting the expression of the TTLL4 gene in a subject-derived cell, which reagent may be selected from the group of:

(a) a reagent for detecting mRNA of the TTLL4 gene;

(b) a reagent for detecting the TTLL4 protein or the immunologically fragment thereof; and

(c) a reagent for detecting the biological activity of the TTLL4 protein.

Examples of reagents suitable for detecting mRNA of the TTLL4 gene include nucleic acids that specifically bind to or identify the TTLL4 mRNA, such as oligonucleotides that have a complementary sequence to a portion of the TTLL4 mRNA. These kinds of oligonucleotides are exemplified by primers and probes that are specific to the TTLL4 mRNA. These kinds of oligonucleotides may be prepared based on methods well known in the art. If needed, the reagent for detecting the TTLL4 mRNA may be immobilized on a solid matrix. Moreover, more than one reagent for detecting the TTLL4 mRNA may be included in the kit.

On the other hand, examples reagents suitable for detecting the TTLL4 protein or the immunologically fragment thereof may include antibodies to the TTLL4 protein or the immunologically fragment thereof. The antibody may be monoclonal or polyclonal. Furthermore, any fragment or modification (e.g., chimeric antibody, scFv, Fab, F(ab′)₂, Fv, etc.) of the antibody may be used as the reagent, so long as the fragment or modified antibody retains the binding ability to the TTLL4 protein or the immunologically fragment thereof. Methods to prepare these kinds of antibodies for the detection of proteins are well known in the art, and any method may be employed in the present invention to prepare such antibodies and equivalents thereof. Furthermore, the antibody may be labeled with signal generating molecules via direct linkage or an indirect labeling technique. Labels and methods for labeling antibodies and detecting the binding of the antibodies to their targets are well known in the art, and any labels and methods may be employed for the present invention. Moreover, more than one reagent for detecting the TTLL4 protein may be included in the kit.

The kit may contain more than one of the aforementioned reagents. The kit can further include a solid matrix and reagent for binding a probe against a TTLL4 gene or antibody against a TTLL4 peptide, a medium and container for culturing cells, positive and negative control reagents, and a secondary antibody for detecting an antibody against a TTLL4 peptide. For example, tissue samples obtained from subjects without cancer or suffering from cancer, may serve as useful control reagents. A kit of the present invention may further include other materials desirable from a commercial and user standpoint, including buffers, diluents, filters, needles, syringes, and package inserts (e.g., written, tape, CD-ROM, etc.) with instructions for use. These reagents and such may be retained in a container with a label. Suitable containers include bottles, vials, and test tubes. The containers may be formed from a variety of materials, such as glass or plastic.

In an embodiment of the present invention, when the reagent is a probe against the TTLL4 mRNA, the reagent may be immobilized on a solid matrix, such as a porous strip, to form at least one detection site. The measurement or detection region of the porous strip may include a plurality of sites, each containing a nucleic acid (probe). A test strip may also contain sites for negative and/or positive controls. Alternatively, control sites may be located on a strip separated from the test strip. Optionally, the different detection sites may contain different amounts of immobilized nucleic acids, i.e., a higher amount in the first detection site and lesser amounts in subsequent sites. Upon the addition of a test sample, the number of sites displaying a detectable signal provides a quantitative indication of the amount of TTLL4 mRNA present in the sample. The detection sites may be configured in any suitably detectable shape and are typically in the shape of a bar or dot spanning the width of a test strip.

The kit of the present invention may further include a positive control sample or TTLL4 standard sample. The positive control sample of the present invention may be prepared by collecting TTLL4 positive samples and then assaying their TTLL4 levels. Alternatively, a purified TTLL4 protein or polynucleotide may be added to cells that do not express TTLL4 to form the positive sample or the TTLL4 standard sample. In the present invention, purified TTLL4 may be a recombinant protein. The TTLL4 level of the positive control sample is, for example, more than the cut off value.

In one embodiment, the present invention further provides a diagnostic kit including, a protein or a partial protein thereof specifically recognized by the antibody of the present invention or the fragment thereof.

Examples of the partial peptide of the protein of the present invention include polypeptides consisting of at least 8, preferably 15, and more preferably 20 contiguous amino acids in the amino acid sequence of the protein of the present invention. Cancer can be diagnosed by detecting an antibody in a sample (e.g., blood, tissue) using a protein or a peptide (polypeptide) of the present invention. The method for preparing the protein of the present invention and peptides are as described above.

The methods for diagnosing cancer of the present invention can be performed by determining the difference between the amount of anti-TTLL4 antibody and that in the corresponding control sample as describe above. The subject is suspected to be suffering from cancer, if cells or tissues of the subject contain antibodies against the expression products (TTLL4) of the gene and the quantity of the anti-TTLL4 antibody is determined to be more than the cut off value in level compared to that in normal control.

In another embodiment, a diagnostic kit of the present invention may include the peptide of the present invention and an HLA molecule binding thereto. The method for detecting antigen specific CTLs using antigenic peptides and HLA molecules has already been established (for example, Altman J D et al., Science. 1996, 274(5284): 94-6). Thus, the complex of the peptide of the present invention and the HLA molecule can be applied to the detection method to detect tumor antigen specific CTLs, thereby enabling earlier detection, recurrence and/or metastasis of cancer. Further, it can be employed for the selection of subjects applicable with the pharmaceuticals including the peptide of the present invention as an active ingredient, or the assessment of the treatment effect of the pharmaceuticals.

Particularly, according to the known method (see, for example, Altman J D et al., Science. 1996, 274(5284): 94-6), the oligomer complex, such as tetramer, of the radiolabeled HLA molecule and the peptide of the present invention can be prepared. With using the complex, the diagnosis can be done, for example, by quantifying the antigen-peptide specific CTLs in the peripheral blood lymphocytes derived from the subject suspected to be suffering from cancer.

The present invention further provides a method or diagnostic agents for evaluating immunological response of subject by using peptide epitopes as described herein. In one embodiment of the invention, HLA-A24 or HLA-A24 restricted peptides as described herein are used as reagents for evaluating or predicting an immune response of a subject. The immune response to be evaluated is induced by contacting an immunogen with immunocompetent cells in vitro or in vivo. In preferred embodiments, the immunocompetent cells for evaluating an immunological response, may be selected from among peripheral blood, peripheral blood lymphocyte (PBL), and peripheral blood mononuclear cell (PBMC). Methods for collecting or isolating such immunocompetent cells are well known in the arts. In some embodiments, any agent that may result in the production of antigen specific CTLs that recognize and bind to the peptide epitope (s) may be employed as the reagent. The peptide reagent need not be used as the immunogen. Assay systems that are used for such an analysis include relatively recent technical developments such as tetramers, staining for intracellular lymphokines and interferon release assays, or ELISPOT assays. In a preferred embodiment, immunocompetent cells to be contacted with peptide reagent may be antigen presenting cells including dendritic cells.

For example, peptides of the present invention may be used in tetramer staining assays to assess peripheral blood mononuclear cells for the presence of antigen-specific CTLs following exposure to a tumor cell antigen or an immunogen. The HLA tetrameric complex may be used to directly visualize antigen specific CTLs (see, e.g., Ogg et al., Science 279: 2103-2106, 1998; and Altman et al, Science 174: 94-96, 1996) and determine the frequency of the antigen-specific CTL population in a sample of peripheral blood mononuclear cells. A tetramer reagent using a peptide of the invention may be generated as described below.

A peptide that binds to an HLA molecule is refolded in the presence of the corresponding HLA heavy chain and beta 2-microglobulin to generate a trimolecular complex. In the complex, carboxyl terminal of the heavy chain is biotinylated at a site that was previously engineered into the protein. Then, streptavidin is added to the complex to form tetramer composed of the trimolecular complex and streptavidin. By means of fluorescently labeled streptavidin, the tetramer can be used to stain antigen-specific cells. The cells can then be identified, for example, by flow cytometry. Such an analysis may be used for diagnostic or prognostic purposes. Cells identified by the procedure can also be used for therapeutic purposes.

The present invention also provides reagents to evaluate immune recall responses (see, e.g., Bertoni etaL., J. Clin. Invest. 100: 503-513, 1997 and Penna et al., J. Exp. Med. 174: 1565-1570, 1991) including peptides of the present invention. For example, patient PBMC samples from individuals with cancer to be treated are analyzed for the presence of antigen-specific CTLs using specific peptides. A blood sample containing mononuclear cells can be evaluated by cultivating the PBMCs and stimulating the cells with a peptide of the invention. After an appropriate cultivation period, the expanded cell population can be analyzed, for example, for CTL activity.

The peptides may be also used as reagents to evaluate the efficacy of a vaccine. PBMCs obtained from a patient vaccinated with an immunogen may be analyzed using, for example, either of the methods described above. The patient is HLA typed, and peptide epitope reagents that recognize the allele specific molecules present in that patient are selected for the analysis. The immunogenicity of the vaccine may be indicated by the presence of epitope-specific CTLs in the PBMC sample.

The peptides of the invention may be also used to make antibodies, using techniques well known in the art (see, e.g. CURRENT PROTOCOLS IN IMMUNOLOGY, Wiley/Greene, NY; and Antibodies A Laboratory Manual, Harlow and Lane, Cold Spring Harbor Laboratory Press, 1989), which may be useful as reagents to diagnose or monitor cancer. Such antibodies may include those that recognize a peptide in the context of an HLA molecule, i.e., antibodies that bind to a peptide-MHC complex.

The peptides and compositions of the present invention have a number of additional uses, some of which are described herein. For instance, the present invention provides a method for diagnosing or detecting a disorder characterized by expression of a TTLL4 immunogenic polypeptide. These methods involve determining expression of a TTLL4 HLA binding peptide, or a complex of a TTLL4 HLA binding peptide and an HLA class I molecule in a biological sample. The expression of a peptide or complex of peptide and HLA class I molecule can be determined or detected by assaying with a binding partner for the peptide or complex. In a preferred embodiment, a binding partner for the peptide or complex is an antibody recognizes and specifically bind to the peptide. The expression of TTLL4 in a biological sample, such as a tumor biopsy, can also be tested by standard PCR amplification protocols using TTLL4 primers. An example of tumor expression is presented herein and further disclosure of exemplary conditions and primers for TTLL4 amplification can be found in WO2003/27322.

Preferably, the diagnostic methods involve contacting a biological sample isolated from a subject with an agent specific for the TTLL4 HLA binding peptide to detect the presence of the TTLL4 HLA binding peptide in the biological sample. As used herein, “contacting” means placing the biological sample in sufficient proximity to the agent and under the appropriate conditions of, e.g., concentration, temperature, time, ionic strength, to allow the specific interaction between the agent and TTLL4 HLA binding peptide that are present in the biological sample. In general, the conditions for contacting the agent with the biological sample are conditions known by those of ordinary skill in the art to facilitate a specific interaction between a molecule and its cognate (e.g., a protein and its receptor cognate, an antibody and its protein antigen cognate, a nucleic acid and its complementary sequence cognate) in a biological sample. Exemplary conditions for facilitating a specific interaction between a molecule and its cognate are described in U.S. Pat. No. 5,108,921, issued to Low et al.

The diagnostic method of the present invention can be performed in either or both of in vivo and in vitro. Accordingly, biological sample can be located in vivo or in vitro in the present invention. For example, the biological sample can be a tissue in vivo and the agent specific for the TTLL4 immunogenic polypeptide can be used to detect the presence of such molecules in the tissue. Alternatively, the biological sample can be collected or isolated in vitro (e.g., a blood sample, tumor biopsy, tissue extract). In a particularly preferred embodiment, the biological sample can be a cell-containing sample, more preferably a sample containing tumor cells collected from a subject to be diagnosed or treated.

Alternatively, the diagnosis can be done, by a method which allows direct quantification of antigen-specific T cells by staining with Fluorescein-labelled HLA multimeric complexes (for example, Altman, J. D. et al., 1996, Science 274: 94; Altman, J. D. et al., 1993, Proc. Natl. Acad. Sci. USA 90:10330). Staining for intracellular lymphokines, and interferon-gamma release assays or ELISPOT assays also has been provided. Tetramer staining, intracellular lymphokine staining and ELISPOT assays all appear to be at least 10-fold more sensitive than more conventional assays (Murali-Krishna, K. et al., 1998, Immunity 8: 177; Lalvani, A. et al., 1997, J. Exp. Med. 186: 859; Dunbar, P. R. et al., 1998, Curr. Biol. 8: 413). Pentamers (e.g., US 2004-209295A), dextramers (e.g., WO 02/072631), and streptamers (e.g., Nature medicine 6. 631-637 (2002)) may also be used.

For instance, in some embodiments, the present invention provides a method for diagnosing or evaluating an immunological response of a subject administered at least one of TTLL4 peptides of the present invention, the method including the steps of:

(a) contacting an immunogen with immunocompetent cells under the condition suitable for induction of CTL specific to the immunogen;

(b) detecting or determining induction level of the CTL induced in step (a); and

(c) correlating the immunological response of the subject with the CTL induction level.

In the present invention, the immunogen is at least one of (a) a TTLL4 peptide selected from among the amino acid sequences of SEQ ID NOs: 1, 3 to 37 and 38 to 73, peptides having such amino acid sequences, and peptides having in which such amino acid sequences have been modified with 1, 2 or more amino acid substitution(s). In the meantime, conditions suitable of induction of immunogen specific CTL are well known in the art. For example, immunocompetent cells may be cultured in vitro under the presence of immunogen(s) to induce immunogen specific CTL. In order to induce immunogen specific CTLs, any stimulating factors may be added to the cell culture. For example, IL-2 is preferable stimulating factors for the CTL induction.

In some embodiments, the step of monitoring or evaluating immunological response of a subject to be treated with peptide cancer therapy may be performed before, during and/or after the treatment. In general, during a protocol of cancer therapy, immunogenic peptides are administered repeatedly to a subject to be treated. For example, immunogenic peptides may be administered every week for 3-10 weeks. Accordingly, the immunological response of the subject can be evaluated or monitored during the cancer therapy protocol. Alternatively, the step of evaluation or monitoring of immunological response to the cancer therapy may at the completion of the therapy protocol.

According to the present invention, enhanced induction of immunogen specific CTL as compared with a control indicates that the subject to be evaluated or diagnosed immunologically responded to the immunogen(s) that has/have been administered. Suitable controls for evaluating the immunological response may include, for example, a CTL induction level when the immunocompetent cells are contacted with no peptide, or control peptide(s) having amino acid sequences other than any TTLL4 peptides. (e.g. random amino acid sequence). In a preferred embodiment, the immunological response of the subject is evaluated in a sequence specific manner, by comparison with an immunological response between each immunogen administered to the subject. In particular, even when a mixture of some kinds of TTLL4 peptides is administered to the subject, immunological response might vary depending on the peptides. In that case, by comparison of the immunological response between each peptide, peptides to which the subject show higher response can be identified.

### XII. ANTIBODIES

The present invention further provides antibodies that bind to peptides of the present invention. Preferred antibodies specifically bind to peptides of the present invention and will not bind (or will bind weakly) to non-peptide of the present invention. Alternatively, antibodies bind to peptides of the invention as well as the homologs thereof. Antibodies against peptides of the invention can find use in cancer diagnostic and prognostic assays, and imaging methodologies. Similarly, such antibodies can find use in the treatment, diagnosis, and/or prognosis of other cancers, to the extent TTLL4 is also expressed or over-expressed in cancer patient. Moreover, intracellularly expressed antibodies (e.g., single chain antibodies) may therapeutically find use in treating cancers in which the expression of TTLL4 is involved, example of which include, but are not limited to, bladder cancer, cholangiocellular carcinoma, CML, colon and rectum cancer, esophageal cancer, liver cancer, lymphoma, pancreatic cancer, prostate cancer, renal carcinoma, SCLC, NSCLC, soft tissue tumor and osteosarcoma.

The present invention also provides various immunological assays for the detection and/or quantification of the TTLL4 protein (SEQ ID NO: 80) or fragments thereof, including polypeptides consisting of amino acid sequences selected from the group consisting of SEQ ID NOs: 1, 3 to 37 and 38 to 73. Such assays may include one or more anti-TTLL4 antibodies capable of recognizing and binding a TTLL4 protein or fragments thereof, as appropriate. In the context of the present invention, anti-TTLL4 antibodies binding to TTLL4 polypeptide preferably recognize polypeptide consisting of amino acid sequences selected from the group consisting of SEQ ID NOs: 1, 3 to 37 and 38 to 73. A binding specificity of antibody can be confirmed with inhibition test. That is, when the binding between an antibody to be analyzed and full-length of TTLL4 polypeptide is inhibited under presence of any fragment polypeptides consisting of amino acid sequence of SEQ ID NOs: 1, 3 to 37 and 38 to 73, it is shown that this antibody specifically binds to the fragment. In the context of the present invention, such immunological assays are performed within various immunological assay formats well known in the art, including but not limited to, various types of radioimmunoassays, immuno-chromatograph technique, enzyme-linked immunosorbent assays (ELISA), enzyme-linked immunofluorescent assays (ELIFA), and the like.

Related immunological but non-antibody assays of the invention may also include T cell immunogenicity assays (inhibitory or stimulatory) as well as MHC binding assays. In addition, the present invention contemplates immunological imaging methods capable of detecting cancers expressing TTLL4, example of which include, but are not limited to, radioscintigraphic imaging methods using labeled antibodies of the present invention. Such assays find clinical use in the detection, monitoring, and prognosis of TTLL4 expressing cancers, examples of which include, but are not limited to, bladder cancer, cholangiocellular carcinoma, CML, colon and rectum cancer, esophageal cancer, liver cancer, lymphoma, pancreatic cancer, prostate cancer, renal carcinoma, SCLC, NSCLC, soft tissue tumor and osteosarcoma.

The present invention also provides antibodies that bind to the peptides of the invention. An antibody of the invention can be used in any form, for example as a monoclonal or polyclonal antibody, and may further include antiserum obtained by immunizing an animal such as a rabbit with the peptide of the invention, all classes of polyclonal and monoclonal antibodies, human antibodies and humanized antibodies produced by genetic recombination.

A peptide of the invention used as an antigen to obtain an antibody may be derived from any animal species, but is preferably derived from a mammal such as a human, mouse, or rat, more preferably from a human. A human-derived peptide may be obtained from the nucleotide or amino acid sequences disclosed herein.

According to the present invention, the peptide to be used as an immunization antigen may be a complete protein or a partial peptide of the protein. A partial peptide may include, for example, the amino (N)-terminal or carboxy (C)-terminal fragment of a peptide of the present invention.

Herein, an antibody is defined as a protein that reacts with either the full length or a fragment of a TTLL4 peptide. In a preferred embodiment, an antibody of the present invention can recognize fragment peptides of TTLL4 having an amino acid sequence selected from the group consisting of SEQ ID NOs: 1, 3 to 37 and 38 to 73. Methods for synthesizing oligopeptide are well known in the arts. After the synthesis, peptides may be optionally purified prior to use as immunogen. In the context of the present invention, the oligopeptide (e.g., 9- or 10 mer) may be conjugated or linked with carriers to enhance the immunogenicity. Keyhole-limpet hemocyanin (KLH) is well known as the carrier. Method for conjugating KLH and peptide are also well known in the arts.

Alternatively, a gene encoding a peptide of the invention or fragment thereof may be inserted into a known expression vector, which is then used to transform a host cell as described herein. The desired peptide or fragment thereof may be recovered from the outside or inside of host cells by any standard method, and may subsequently be used as an antigen. Alternatively, whole cells expressing the peptide or their lysates or a chemically synthesized peptide may be used as the antigen.

Any mammalian animal may be immunized with the antigen, but preferably the compatibility with parental cells used for cell fusion is taken into account. In general, animals of Rodentia, Lagomorpha or Primates may be used. Animals of the family Rodentia include, for example, mouse, rat and hamster. Animals of the family Lagomorpha include, for example, rabbit. Animals of the Primate family include, for example, a monkey of Catarrhini (old world monkey) such as Macaca fascicularis, rhesus monkey, sacred baboon and chimpanzees.

Methods for immunizing animals with antigens are known in the art. Intraperitoneal injection or subcutaneous injection of antigens is a standard method for the immunization of mammals. More specifically, antigens may be diluted and suspended in an appropriate amount of phosphate buffered saline (PBS), physiological saline, etc. If desired, the antigen suspension may be mixed with an appropriate amount of a standard adjuvant, such as Freund's complete adjuvant, made into emulsion and then administered to mammalian animals. Preferably, it is followed by several administrations of antigen mixed with an appropriately amount of Freund's incomplete adjuvant every 4 to 21 days. An appropriate carrier may also be used for immunization. After immunization as above, serum may be examined by a standard method for an increase in the amount of desired antibodies.

Polyclonal antibodies against the peptides of the present invention may be prepared by collecting blood from the immunized mammal examined for the increase of desired antibodies in the serum, and by separating serum from the blood by any conventional method. Polyclonal antibodies may include serum containing the polyclonal antibodies, as well as the fraction containing the polyclonal antibodies may be isolated from the serum. Immunoglobulin G or M can be prepared from a fraction which recognizes only the peptide of the present invention using, for example, an affinity column coupled with the peptide of the present invention, and further purifying this fraction using protein A or protein G column.

To prepare monoclonal antibodies, immune cells are collected from the mammal immunized with the antigen and checked for the increased level of desired antibodies in the serum as described above, and are subjected to cell fusion. The immune cells used for cell fusion may preferably be obtained from spleen. Other preferred parental cells to be fused with the above immunocyte include, for example, myeloma cells of mammalians, and more preferably myeloma cells having an acquired property for the selection of fused cells by drugs.

The above immunocyte and myeloma cells can be fused according to known methods, for example, the method of Milstein et al. (Galfre and Milstein, Methods Enzymol 73: 3-46 (1981)).

Resulting hybridomas obtained by the cell fusion may be selected by cultivating them in a standard selection medium, such as HAT medium (hypoxanthine, aminopterin and thymidine containing medium). The cell culture is typically continued in the HAT medium for several days to several weeks, the time being sufficient to allow all the other cells, with the exception of the desired hybridoma (non-fused cells), to die. Then, the standard limiting dilution may be performed to screen and clone a hybridoma cell producing the desired antibody.

In addition to the above method, in which a non-human animal is immunized with an antigen for preparing hybridoma, human lymphocytes such as those infected by EB virus may be immunized with a peptide, peptide expressing cells or their lysates in vitro. Then, the immunized lymphocytes are fused with human-derived myeloma cells that are capable of indefinitely dividing, such as U266, to yield a hybridoma producing a desired human antibody that is able to bind to the peptide can be obtained (Unexamined Published Japanese Patent Application No. Sho 63-17688).

The obtained hybridomas are subsequently transplanted into the abdominal cavity of a mouse and the ascites are extracted. The obtained monoclonal antibodies can be purified by, for example, ammonium sulfate precipitation, a protein A or protein G column, DEAE ion exchange chromatography or an affinity column to which the peptide of the present invention is coupled. The antibody of the present invention can be used not only for purification and detection of the peptide of the present invention, but also as a candidate for agonists and antagonists of the peptide of the present invention.

Alternatively, an immune cell, such as an immunized lymphocyte, producing antibodies may be immortalized by an oncogene and used for preparing monoclonal antibodies.

Monoclonal antibodies thus obtained can be also recombinantly prepared using genetic engineering techniques (see, for example, Borrebaeck and Larrick, Therapeutic Monoclonal Antibodies, published in the United Kingdom by MacMillan Publishers LTD (1990)). For example, a DNA encoding an antibody may be cloned from an immune cell, such as a hybridoma or an immunized lymphocyte producing the antibody, inserted into an appropriate vector, and introduced into host cells to prepare a recombinant antibody. The present invention also provides recombinant antibodies prepared as described above.

Furthermore, an antibody of the present invention may be a fragment of an antibody or modified antibody, so long as it binds to one or more of the peptides of the invention. For instance, the antibody fragment may be Fab, F(ab′)₂, Fv or single chain Fv (scFv), in which Fv fragments from H and L chains are ligated by an appropriate linker (Huston et al., Proc Natl Acad Sci USA 85: 5879-83 (1988)). More specifically, an antibody fragment may be generated by treating an antibody with an enzyme, such as papain or pepsin. Alternatively, a gene encoding the antibody fragment may be constructed, inserted into an expression vector and expressed in an appropriate host cell (see, for example, Co et al., J Immunol 152: 2968-76 (1994); Better and Horwitz, Methods Enzymol 178: 476-96 (1989); Pluckthun and Skerra, Methods Enzymol 178: 497-515 (1989); Lamoyi, Methods Enzymol 121: 652-63 (1986); Rousseaux et al., Methods Enzymol 121: 663-9 (1986); Bird and Walker, Trends Biotechnol 9: 132-7 (1991)).

An antibody may be modified by conjugation with a variety of molecules, such as polyethylene glycol (PEG). The present invention provides for such modified antibodies. The modified antibody can be obtained by chemically modifying an antibody. These modification methods are conventional in the field.

Alternatively, an antibody of the present invention may be obtained as a chimeric antibody, between a variable region derived from nonhuman antibody and the constant region derived from human antibody, or as a humanized antibody, including the complementarity determining region (CDR) derived from nonhuman antibody, the frame work region (FR) and the constant region derived from human antibody. Such antibodies can be prepared according to known technology. Humanization can be performed by substituting rodent CDRs or CDR sequences for the corresponding sequences of a human antibody (see, e.g., Verhoeyen et al., Science 239:1534-1536 (1988)). Accordingly, such humanized antibodies are chimeric antibodies, wherein substantially less than an intact human variable domain has been substituted by the corresponding sequence from a non-human species.

Fully human antibodies including human variable regions in addition to human framework and constant regions can also be used. Such antibodies can be produced using various techniques known in the art. For example, in vitro methods involve use of recombinant libraries of human antibody fragments displayed on bacteriophage (e.g., Hoogenboom & Winter, J. Mol. Biol. 227:381 (1991). Similarly, human antibodies can be made by introducing of human immunoglobulin loci into transgenic animals, e.g., mice in which the endogenous immunoglobulin genes have been partially or completely inactivated. This approach is described, e.g., in U.S. Pat. Nos. 6,150,584, 5,545,807; 5,545,806; 5,569,825; 5,625,126; 5,633,425; 5,661,016.

Antibodies obtained as above may be purified to homogeneity. For example, the separation and purification of the antibody can be performed according to the separation and purification methods used for general proteins. For example, the antibody may be separated and isolated by the appropriately selected and combined use of column chromatographies, such as affinity chromatography, filter, ultrafiltration, salting-out, dialysis, SDS polyacrylamide gel electrophoresis and isoelectric focusing (Antibodies: A Laboratory Manual. Ed Harlow and David Lane, Cold Spring Harbor Laboratory (1988)), but are not limited thereto. A protein A column and protein G column can be used as the affinity column. Exemplary protein A columns to be used include, for example, Hyper D, POROS and Sepharose F.F. (Pharmacia).

Exemplary chromatography, with the exception of affinity includes, for example, ion-exchange chromatography, hydrophobic chromatography, gel filtration, reverse phase chromatography, adsorption chromatography and the like (Strategies for Protein Purification and Characterization: A Laboratory Course Manual. Ed Daniel R. Marshak et al., Cold Spring Harbor Laboratory Press (1996)). The chromatographic procedures can be carried out by liquid-phase chromatography, such as HPLC and FPLC.

For example, measurement of absorbance, enzyme-linked immunosorbent assay (ELISA), enzyme immunoassay (EIA), radioimmunoassay (RIA) and/or immunofluorescence may be used to measure the antigen binding activity of the antibody of the invention. In ELISA, the antibody of the present invention is immobilized on a plate, a peptide of the invention is applied to the plate, and then a sample containing a desired antibody, such as culture supernatant of antibody producing cells or purified antibodies, is applied. Then, a secondary antibody that recognizes the primary antibody and is labeled with an enzyme, such as alkaline phosphatase, is applied, and the plate is incubated. Next, after washing, an enzyme substrate, such as p-nitrophenyl phosphate, is added to the plate, and the absorbance is measured to evaluate the antigen binding activity of the sample. A fragment of the peptide, such as a C-terminal or N-terminal fragment, may be used as the antigen to evaluate the binding activity of the antibody. BIAcore (Pharmacia) may be used to evaluate the activity of the antibody according to the present invention.

The above methods allow for the detection or measurement of a peptide of the invention, by exposing an antibody of the invention to a sample presumed to contain a peptide of the invention, and detecting or measuring the immune complex formed by the antibody and the peptide.

Because the method of detection or measurement of the peptide according to the invention can specifically detect or measure a peptide, the method can find use in a variety of experiments in which the peptide is used.

### XIII. VECTORS AND HOST CELLS

The present invention also provides a vector and host cell into which a nucleotide encoding the peptide of the present invention is introduced. A vector of the present invention may be used to keep a nucleotide, especially a DNA, of the present invention in host cell, to express a peptide of the present invention, or to administer a nucleotide of the present invention for gene therapy.

When E. coli is a host cell and the vector is amplified and produced in a large amount in E. coli (e.g., JM109, DH5 alpha, HB101 or XL1Blue), the vector should have “ori” to be amplified in E. coli and a marker gene for selecting transformed E. coli (e.g., a drug-resistance gene selected by a drug such as ampicillin, tetracycline, kanamycin, chloramphenicol or the like). For example, M13-series vectors, pUC-series vectors, pBR322, pBluescript, pCR-Script, etc., can be used. In addition, pGEM-T, pDIRECT and pT7 can also be used for subcloning and extracting cDNA as well as the vectors described above. When a vector is used to produce the protein of the present invention, an expression vector can find use. For example, an expression vector to be expressed in E. coli should have the above characteristics to be amplified in E. coli. When E. coli, such as JM109, DH5 alpha, HB101 or XL1 Blue, are used as a host cell, the vector should have a promoter, for example, lacZ promoter (Ward et al., Nature 341: 544-6 (1989); FASEB J 6: 2422-7 (1992)), araB promoter (Better et al., Science 240: 1041-3 (1988)), T7 promoter or the like, that can efficiently express the desired gene in E. coli. In that respect, pGEX-5X-1 (Pharmacia), “QIAexpress system” (Qiagen), pEGFP and pET (in this case, the host is preferably BL21 which expresses T7 RNA polymerase), for example, can be used instead of the above vectors. Additionally, the vector may also contain a signal sequence for peptide secretion. An exemplary signal sequence that directs the peptide to be secreted to the periplasm of the E. coli is the pelB signal sequence (Lei et al., J Bacteriol 169: 4379 (1987)). Means for introducing of the vectors into the target host cells include, for example, the calcium chloride method, and the electroporation method.

In addition to E. coli, for example, expression vectors derived from mammals (for example, pcDNA3 (Invitrogen) and pEGF-BOS (Nucleic Acids Res 18(17): 5322 (1990)), pEF, pCDM8), expression vectors derived from insect cells (for example, “Bac-to-BAC baculovirus expression system” (GIBCO BRL), pBacPAK8), expression vectors derived from plants (e.g., pMH1, pMH2), expression vectors derived from animal viruses (e.g., pHSV, pMV, pAdexLcw), expression vectors derived from retroviruses (e.g., pZIpneo), expression vector derived from yeast (e.g., “Pichia Expression Kit” (Invitrogen), pNV11, SP-Q01) and expression vectors derived from Bacillus subtilis (e.g., pPL608, pKTH50) can be used for producing the polypeptide of the present invention.

In order to express the vector in animal cells, such as CHO, COS or NIH3T3 cells, the vector should have a promoter necessary for expression in such cells, for example, the SV40 promoter (Mulligan et al., Nature 277: 108 (1979)), the MMLV-LTR promoter, the EF1 alpha promoter (Mizushima et al., Nucleic Acids Res 18: 5322 (1990)), the CMV promoter and the like, and preferably a marker gene for selecting transformants (for example, a drug resistance gene selected by a drug (e.g., neomycin, G418)). Examples of known vectors with these characteristics include, for example, pMAM, pDR2, pBK-RSV, pBK-CMV, pOPRSV and pOP13.

Although methods and materials similar or equivalent to those described herein can be used in the practice or testing of the present invention, suitable methods and materials are described. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Experimental 1

Materials and Methods

Cell Lines

TISI, HLA-A*2402-positive B-lymphoblastoid cell line, was purchased from the IHWG Cell and Gene Bank (Seattle, Wash.). COS7, African green monkey kidney cell line, was purchased from ATCC.

Candidate Selection of Peptides Derived from TTLL4

9-mer and 10-mer peptides derived from TTLL4 that bind to HLA-A*2402 molecule were predicted using “NetMHC3.0” binding prediction server (http://www.cbs.dtu.dk/services/NetMHC/) (Buus et al. (Tissue Antigens., 62:378-84, 2003), Nielsen et al. (Protein Sci., 12:1007-17, 2003, Bioinformatics, 20(9):1388-97, 2004)). These peptides were synthesized by Biosynthesis (Lewisville, Tex.) according to a standard solid phase synthesis method and purified by reversed phase high performance liquid chromatography (HPLC). The purity (>90%) and the identity of the peptides were determined by analytical HPLC and mass spectrometry analysis, respectively. Peptides were dissolved in dimethylsulfoxide at 20 mg/ml and stored at −80 degrees C.

In Vitro CTL Induction

Monocyte-derived dendritic cells (DCs) were used as antigen-presenting cells to induce cytotoxic T lymphocyte (CTL) responses against peptides presented on human leukocyte antigen (HLA). DCs were generated in vitro as described elsewhere (Nakahara S et al., Cancer Res 2003, 63(14): 4112-8). Specifically, peripheral blood mononuclear cells isolated from a normal volunteer (HLA-A*2402 positive) by Ficoll-Plaque (Pharmacia) solution were separated by adherence to a plastic tissue culture dish (Becton Dickinson) so as to enrich them as the monocyte fraction. The monocyte-enriched population was cultured in the presence of 1000 U/ml of granulocyte-macrophage colony-stimulating factor (R&D System) and 1000 U/ml of interleukin (IL)-4 (R&D System) in AIM-V Medium (Invitrogen) containing 2% heat-inactivated autologous serum (AS). After 7 days of culture, the cytokine-induced DCs were pulsed with 20 micro-g/ml of each of the synthesized peptides in the presence of 3 micro-g/ml of beta 2-microglobulin for 3 hr at 37 degrees C. in AIM-V Medium. The generated cells appeared to express DC-associated molecules, such as CD80, CD83, CD86 and HLA class II, on their cell surfaces (data not shown). These peptide-pulsed DCs were then inactivated by X-irradiation (20 Gy) and mixing at a 1:20 ratio with autologous CD8+ T cells, obtained by positive selection with CD8 Positive Isolation Kit (Dynal). These cultures were set up in 48-well plates (Corning); each well contained 1.5×10⁴ peptide-pulsed DCs, 3×10⁵ CD8+ T cells and 10 ng/ml of IL-7 (R&D System) in 0.5 ml of AIM-V/2% AS medium. Three days later, these cultures were supplemented with IL-2 (CHIRON) to a final concentration of 20 IU/ml. On day 7 and 14, the T cells were further stimulated with the autologous peptide-pulsed DCs. The DCs were prepared each time by the same way described above. CTL was tested against peptide-pulsed TISI cells after the 3rd round of peptide stimulation on day 21 (Tanaka H et al., Br J Cancer 2001, 84(1): 94-9; Umano Y et al., Br J Cancer 2001, 84(8): 1052-7; Uchida N et al., Clin Cancer Res 2004, 10(24): 8577-86; Suda T et al., Cancer Sci 2006, 97(5): 411-9; Watanabe T et al., Cancer Sci 2005, 96(8): 498-506).

CTL Expansion Procedure

CTLs were expanded in culture using the method similar to the one described by Riddell et al. (Walter E A et al., N Engl J Med 1995 333(16): 1038-44; Riddell S R et al., Nat Med 1996, 2(2): 216-23). A total of 5×10⁴ CTLs were suspended in 25 ml of AIM-V/5% AS medium with 2 kinds of human B-lymphoblastoid cell lines, inactivated by Mitomycin C, in the presence of 40 ng/ml of anti-CD3 monoclonal antibody (Pharmingen). One day after initiating the cultures, 120 IU/ml of IL-2 were added to the cultures. The cultures were fed with fresh AIM-V/5% AS medium containing 30 IU/ml of IL-2 on days 5, 8 and 11 (Tanaka H et al., Br J Cancer 2001, 84(1): 94-9; Umano Y et al., Br J Cancer 2001, 84(8): 1052-7; Uchida N et al., Clin Cancer Res 2004, 10(24): 8577-86; Suda T et al., Cancer Sci 2006, 97(5): 411-9; Watanabe T et al., Cancer Sci 2005, 96(8): 498-506).

Establishment of CTL Clones

The dilutions were made to have 0.3, 1, and 3 CTLs/well in 96 round-bottomed micro titer plate (Nalge Nunc International). CTLs were cultured with 1×10⁴ cells/well of 2 kinds of human B-lymphoblastoid cell lines, 30 ng/ml of anti-CD3 antibody, and 125 U/ml of IL-2 in a total of 150 micro-Dwell of AIM-V Medium containing 5% AS. 50 micro-l/well of IL-2 were added to the medium 10 days later so to reach a final concentration of 125 U/ml IL-2. CTL activity was tested on the 14th day, and CTL clones were expanded using the same method as described above (Uchida N et al., Clin Cancer Res 2004, 10(24): 8577-86; Suda T et al., Cancer Sci 2006, 97(5): 411-9; Watanabe T et al., Cancer Sci 2005, 96(8): 498-506).

Specific CTL Activity

To examine specific CTL activity, interferon (IFN)-gamma enzyme-linked immunospot (ELISPOT) assay and IFN-gamma enzyme-linked immunosorbent assay (ELISA) were performed. Specifically, peptide-pulsed TISI (1×10⁴/well) was prepared as stimulator cells. Cultured cells in 48 wells were used as responder cells. IFN-gamma ELISPOT assay and IFN-gamma ELISA assay were performed under manufacture procedure.

Establishment of the Cells Forcibly Expressing Either or Both of the Target Gene and HLA-A24

The cDNA encoding an open reading frame of target genes or HLA-A*2402 was amplified by PCR. The PCR-amplified product was cloned into a vector. The plasmids were transfected into COS7, which is the target genes and HLA-A*2402-null cell line, using lipofectamine 2000 (Invitrogen) according to the manufacturer's recommended procedures. After 2 days from transfection, the transfected cells were harvested with versene (Invitrogen) and used as the target cells (5×10⁴ cells/well) for CTL activity assay.

Results

Prediction of HLA-A24 Binding Peptides Derived from TTLL4

Table 1a and 1b show the HLA-A24 binding 9 mer and 10 mer peptides of TTLL4 in the order of high binding affinity. A total of 37 peptides with potential HLA-A24 binding ability were selected and examined to determine the epitope peptides.

TABLE 1a
HLA-A24 binding 9mer peptides derived from TTLL4

| Start Position | amino acid sequence | Kd (nM) | SEQ ID NO |
|---|---|---|---|
| 750 | RYLHKPYLI | 5 | 1 |
| 579 | LFPNVPPTI | 21 | 2 |
| 994 | FYASVLDVL | 24 | 3 |
| 769 | VYVTSYDPL | 37 | 4 |
| 755 | PYLISGSKF | 43 | 5 |
| 79 | AYFFCPSTL | 54 | 6 |
| 684 | RFGKKEFSF | 68 | 7 |
| 689 | EFSFFPQSF | 77 | 8 |
| 779 | IYLFSDGLV | 119 | 9 |
| 304 | WYNRNNLAM | 229 | 10 |
| 793 | KYSPSMKSL | 284 | 11 |
| 691 | SFFPQSFIL | 325 | 12 |
| 41 | VWPQAHQQV | 387 | 13 |
| 1086 | VWSLPTSLL | 399 | 14 |
| 1186 | TFQSISDSL | 473 | 15 |
| 103 | CYLHSLPDL | 492 | 16 |
| 362 | SFLNPSFQW | 754 | 17 |
| 1037 | RFFEQPRYF | 800 | 18 |
| 773 | SYDPLRIYL | 1501 | 19 |

TABLE 1b
HLA-A24 binding 10mer peptides derived from TTLL4

| Start Position | amino acid sequence | Kd (nM) | SEQ ID NO |
|---|---|---|---|
| 103 | CYLHSLPDLF | 11 | 20 |
| 773 | SYDPLRIYLF | 21 | 21 |
| 883 | PYSCHELFGF | 47 | 22 |
| 127 | PYQQLESFCL | 67 | 23 |
| 684 | RFGKKEFSFF | 70 | 24 |
| 1043 | RYFNILTTQW | 148 | 25 |
| 223 | MWPNSTPVPL | 181 | 26 |
| 122 | SYRQKPYQQL | 210 | 27 |
| 1186 | TFQSISDSLL | 323 | 28 |
| 1022 | QFERIFPSHI | 561 | 29 |
| 689 | EFSFFPQSFI | 584 | 30 |
| 804 | KFMHLTNYSV | 836 | 31 |
| 994 | FYASVLDVLT | 860 | 32 |
| 993 | DFYASVLDVL | 3998 | 33 |
| 1105 | AFSKSETSKL | 5879 | 34 |
| 696 | SFILPQDAKL | 7815 | 35 |
| 665 | SFQIGRKDRL | 18177 | 36 |
| 891 | GFDIMLDENL | 24816 | 37 |

Start position indicates the number of amino acid residue from the N-terminus of TTLL4.

Dissociation constant [Kd (nM)] is derived from “NetMHC3.0”.

CTL Induction with the Predicted Peptides from TTLL4 Restricted with HLA-A*2402

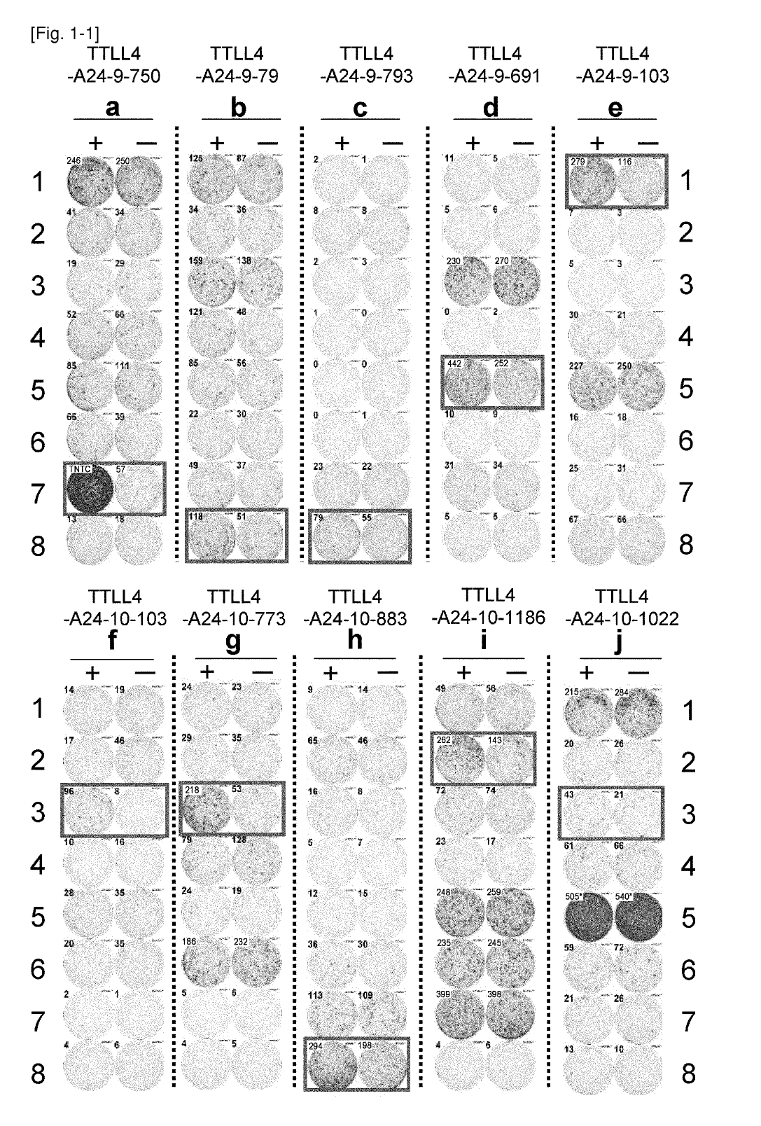

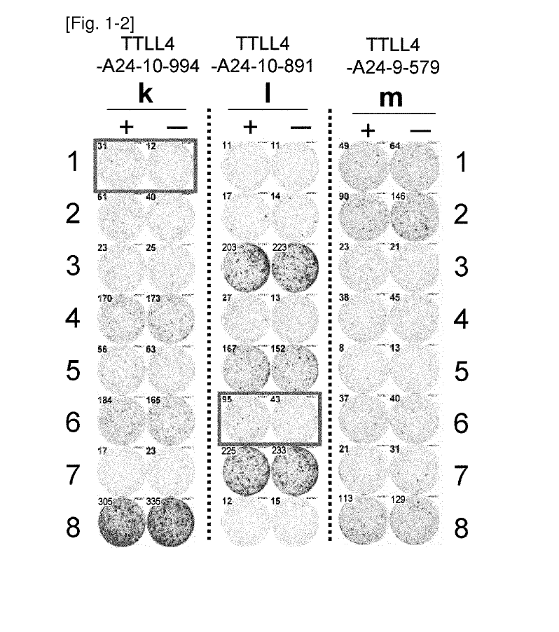

CTLs for those peptides derived from TTLL4 were generated according to the protocols as described in “Materials and Methods”. Peptide specific CTL activity was determined by IFN-gamma ELISPOT assay (FIG. 1a-1). It showed that the well number #7 with TTLL4-A24-9-750 (SEQ ID NO:1) (a), #8 with TTLL4-A24-9-79 (SEQ ID NO:6) (b), #8 with TTLL4-A24-9-793 (SEQ ID NO:11) (c), #5 with TTLL4-A24-9-691 (SEQ ID NO:12) (d), #1 with TTLL4-A24-9-103 (SEQ ID NO:16) (e), #3 with TTLL4-A24-10-103 (SEQ ID NO:20) (f), #3 with TTLL4-A24-10-773 (SEQ ID NO:21) (g), #8 with TTLL4-A24-10-883 (SEQ ID NO:22) (h), #2 with TTLL4-A24-10-1186 (SEQ ID NO:28) (i), #3 with TTLL4-A24-10-1022 (SEQ ID NO:29) (j), #1 with TTLL4-A24-10-994 (SEQ ID NO:32) (k) and #6 with TTLL4-A24-10-891 (SEQ ID NO:37) (1) demonstrated potent IFN-gamma production as compared to the control wells. On the other hand, no specific CTL activity was determined by stimulation with other peptides shown in Table 1a and 1b, despite those peptides had possible binding activity with HLA-A*2402. As a typical case of negative data, it was not shown specific IFN-gamma production from the CTL stimulated with TTLL4-A24-9-579 (SEQ ID NO: 2) (m). As a result, it indicated that 12 peptides derived from TTLL4 were screened as the peptides that could induce potent CTLs.

Establishment of CTL Lines and Clones Against TTLL4 Derived Peptide

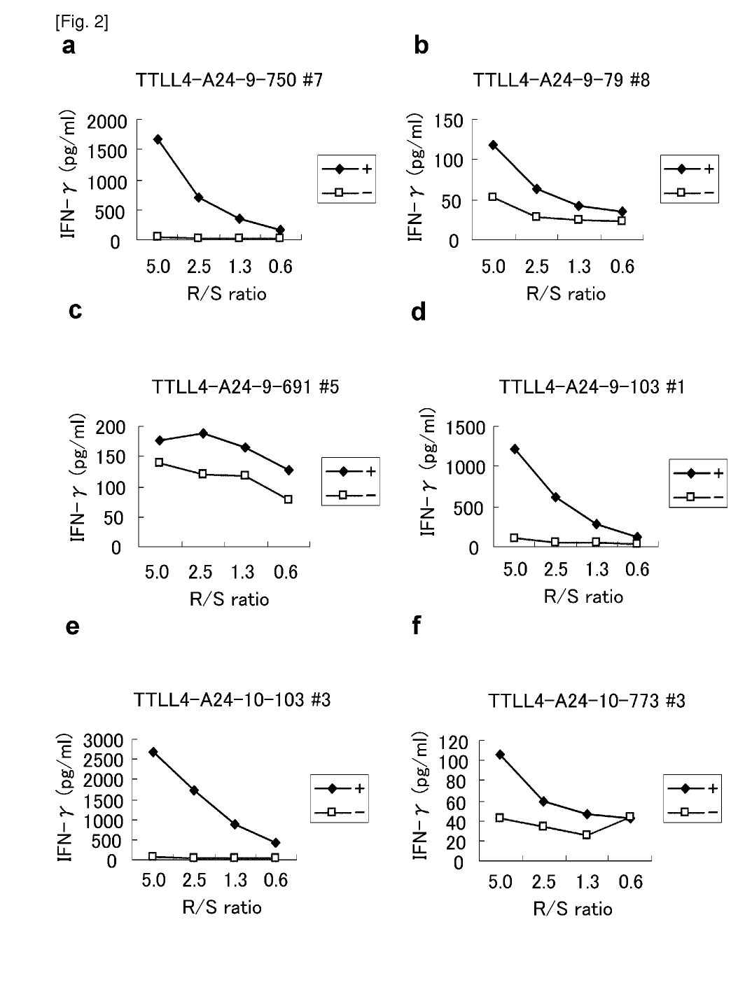

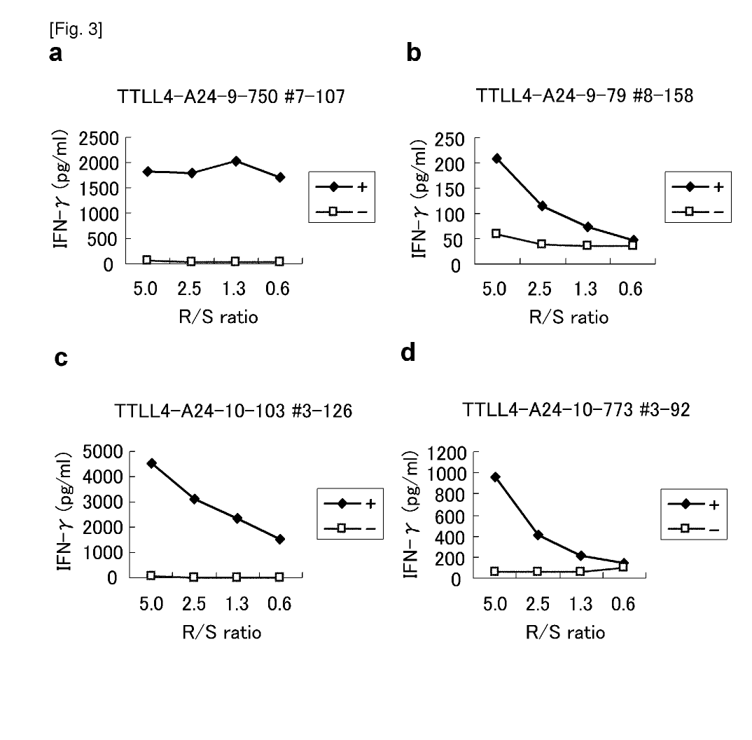

The cells that showed peptide specific CTL activity detected by IFN-gamma ELISPOT assay in the well number #7 with TTLL4-A24-9-750 (SEQ ID NO:1) (a), #8 with TTLL4-A24-9-79 (SEQ ID NO:6) (b), #5 with TTLL4-A24-9-691 (SEQ ID NO:12) (c), #1 with TTLL4-A24-9-103 (SEQ ID NO:16) (d), #3 with TTLL4-A24-10-103 (SEQ ID NO:20) (e) and #3 with TTLL4-A24-10-773 (SEQ ID NO:21) (f). were expanded and CTL lines were established as described in the “Materials and Methods” section above. CTL activity of these CTL lines was determined by IFN-gamma ELISA assay (FIG. 2a-f). CTL lines demonstrated potent IFN-gamma production against the target cells pulsed with the corresponding peptide as compared to target cells without peptide pulse. Furthermore, the CTL clones were established by limiting dilution from the CTL lines as described in “Materials and Methods”, and IFN-gamma production from the CTL clones against target cells pulsed peptide was determined by IFN-gamma ELISA assay. Potent IFN-gamma productions were determined from the CTL clones stimulated with TTLL4-A24-9-750 (SEQ ID NO:1) (a), TTLL4-A24-9-79 (SEQ ID NO:6) (b), TTLL4-A24-10-103 (SEQ ID NO:20) (c) and TTLL4-A24-10-773 (SEQ ID NO:21) (d). (FIG. 3a-d).

Specific CTL Activity Against Target Cells Expressing TTLL4 and HLA-A*2402

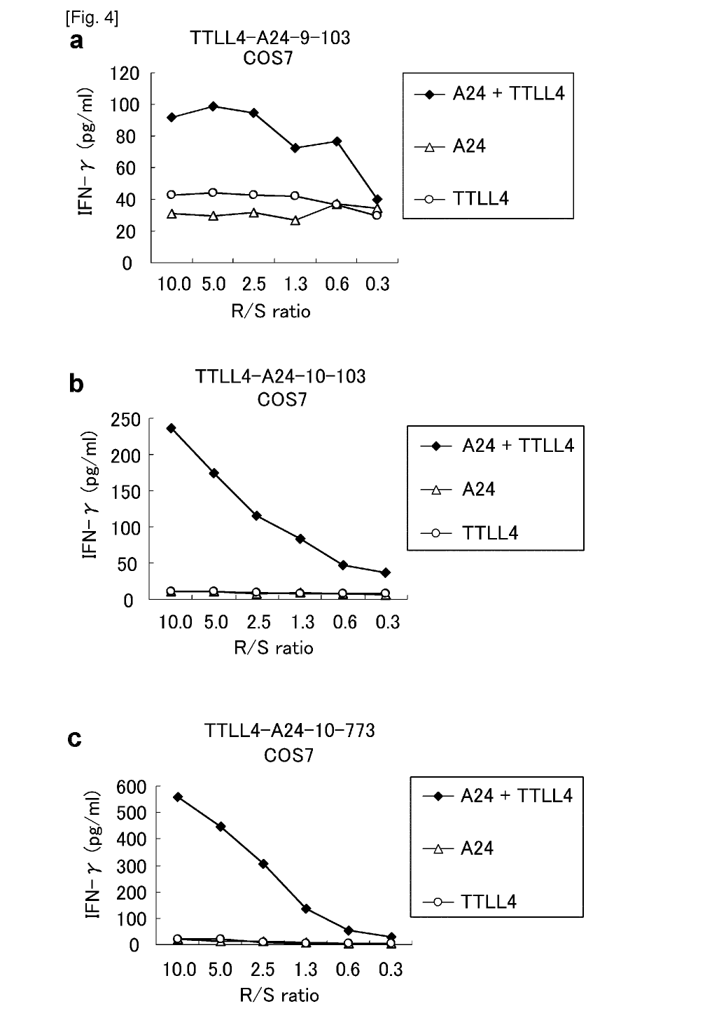

The established CTL lines and clones raised against each peptide were examined for the ability to recognize target cells that express TTLL4 and HLA-A*2402 molecule. Specific CTL activity against COS7 cells which transfected with both the full length of TTLL4 and HLA-A*2402 gene (a specific model for the responder cells that express TTLL4 and HLA-A*2402 gene) was tested by using the CTL lines and clones raised by corresponding peptide as the stimulator cells. COS7 cells transfected with either full length of TTLL4 or HLA-A*2402 were prepared as the controls. In FIG. 4, the CTL line stimulated with TTLL4-A24-9-103 (SEQ ID NO: 16) (a) and the CTL clones stimulated with TTLL4-A24-10-103 (SEQ ID NO: 20) (b) and TTLL4-A24-10-773 (SEQ ID NO: 21) (c) showed potent CTL activity against COS7 cells expressing both TTLL4 and HLA-A*2402. On the other hand, no significant specific CTL activity was detected against the controls. Thus, these data clearly demonstrated that peptides of TTLL4-A24-9-103 (SEQ ID NO: 16) (a), TTLL4-A24-10-103 (SEQ ID NO: 20) (b) and TTLL4-A24-10-773 (SEQ ID NO: 21) (c) were endogenously processed and presented on the target cells with HLA-A*2402 molecule and were recognized by the CTLs. These results indicated that these peptides derived from TTLL4 may be suitable as a cancer vaccine for the treatment of patients with TTLL4 expressing tumors.

Homology Analysis of Antigen Peptides

The CTLs stimulated with TTLL4-A24-9-750 (SEQ ID NO:1), TTLL4-A24-9-79 (SEQ ID NO:6), TTLL4-A24-9-793 (SEQ ID NO:11), TTLL4-A24-9-691 (SEQ ID NO:12), TTLL4-A24-9-103 (SEQ ID NO:16), TTLL4-A24-10-103 (SEQ ID NO:20), TTLL4-A24-10-773 (SEQ ID NO:21), TTLL4-A24-10-883 (SEQ ID NO:22), TTLL4-A24-10-1186 (SEQ ID NO:28), TTLL4-A24-10-1022 (SEQ ID NO:29), TTLL4-A24-10-994 (SEQ ID NO:32) and TTLL4-A24-10-891 (SEQ ID NO:37) showed significant and specific CTL activity. This result may be due to the fact that the sequence of TTLL4-A24-9-750 (SEQ ID NO:1), TTLL4-A24-9-79 (SEQ ID NO:6), TTLL4-A24-9-793 (SEQ ID NO:11), TTLL4-A24-9-691 (SEQ ID NO:12), TTLL4-A24-9-103 (SEQ ID NO:16), TTLL4-A24-10-103 (SEQ ID NO:20), TTLL4-A24-10-773 (SEQ ID NO:21), TTLL4-A24-10-883 (SEQ ID NO:22), TTLL4-A24-10-1186 (SEQ ID NO:28), TTLL4-A24-10-1022 (SEQ ID NO:29), TTLL4-A24-10-994 (SEQ ID NO:32) and TTLL4-A24-10-891 (SEQ ID NO:37) are homologous to peptide derived from other molecules that are known to sensitize the human immune system. To exclude this possibility, homology analysis were performed for this peptide sequence using as queries the BLAST algorithm (http://www.ncbi.nlm.nih.gov/blast/blast.cgi) which revealed no sequence with significant homology. The results of homology analysis indicate that the sequence of TTLL4-A24-9-750 (SEQ ID NO:1), TTLL4-A24-9-79 (SEQ ID NO:6), TTLL4-A24-9-793 (SEQ ID NO:11), TTLL4-A24-9-691 (SEQ ID NO:12), TTLL4-A24-9-103 (SEQ ID NO:16), TTLL4-A24-10-103 (SEQ ID NO:20), TTLL4-A24-10-773 (SEQ ID NO:21), TTLL4-A24-10-883 (SEQ ID NO:22), TTLL4-A24-10-1186 (SEQ ID NO:28), TTLL4-A24-10-1022 (SEQ ID NO:29), TTLL4-A24-10-994 (SEQ ID NO:32) and TTLL4-A24-10-891 (SEQ ID NO:37) are unique and thus, there is little possibility, to our best knowledge, that this molecules raise unintended immunologic response to some unrelated molecule.

In conclusion, novel HLA-A*2402 epitope peptides derived from TTLL4 were identified and demonstrated to be suitable for cancer immunotherapy.

### Experimental 2

Materials and Methods

Cell Lines

T2, HLA-A*0201-positive B-lymphoblastoid cell line, and COST, African green monkey kidney cell line, were purchased from ATCC.

Candidate Selection of Peptides Derived from TTLL4

9-mer and 10-mer peptides derived from TTLL4 that bind to HLA-A*0201 molecule were predicted using “NetMHC3.0” binding prediction server (http://www.cbs.dtu.dk/services/NetMHC/) (Buus et al. (Tissue Antigens., 62:378-84, 2003), Nielsen et al. (Protein Sci., 12:1007-17, 2003, Bioinformatics, 20(9):1388-97, 2004)). These peptides were synthesized by Biosynthesis (Lewisville, Tex.) according to a standard solid phase synthesis method and purified by reversed phase high performance liquid chromatography (HPLC). The purity (>90%) and the identity of the peptides were determined by analytical HPLC and mass spectrometry analysis, respectively. Peptides were dissolved in dimethylsulfoxide at 20 mg/ml and stored at −80 degrees C.

In Vitro CTL Induction

Monocyte-derived dendritic cells (DCs) were used as antigen-presenting cells to induce cytotoxic T lymphocyte (CTL) responses against peptides presented on human leukocyte antigen (HLA). DCs were generated in vitro as described elsewhere (Nakahara S et al., Cancer Res 2003, 63(14): 4112-8). Specifically, peripheral blood mononuclear cells isolated from a normal volunteer (HLA-A*0201 positive) by Ficoll-Plaque (Pharmacia) solution were separated by adherence to a plastic tissue culture dish (Becton Dickinson) so as to enrich them as the monocyte fraction. The monocyte-enriched population was cultured in the presence of 1000 U/ml of granulocyte-macrophage colony-stimulating factor (R&D System) and 1000 U/ml of interleukin (IL)-4 (R&D System) in AIM-V Medium (Invitrogen) containing 2% heat-inactivated autologous serum (AS). After 7 days of culture, the cytokine-induced DCs were pulsed with 20 micro-g/ml of each of the synthesized peptides in the presence of 3 micro-g/ml of beta 2-microglobulin for 3 hr at 37 degrees C. in AIM-V Medium. The generated cells appeared to express DC-associated molecules, such as CD80, CD83, CD86 and HLA class II, on their cell surfaces (data not shown). These peptide-pulsed DCs were then inactivated by X-irradiation (20 Gy) and mixing at a 1:20 ratio with autologous CD8+ T cells, obtained by positive selection with CD8 Positive Isolation Kit (Dynal). These cultures were set up in 48-well plates (Corning); each well contained 1.5×10⁴ peptide-pulsed DCs, 3×10⁵ CD8+ T cells and 10 ng/ml of IL-7 (R&D System) in 0.5 ml of AIM-V/2% AS medium. Three days later, these cultures were supplemented with IL-2 (CHIRON) to a final concentration of 20 IU/ml. On day 7 and 14, the T cells were further stimulated with the autologous peptide-pulsed DCs. The DCs were prepared each time by the same way described above. CTL was tested against peptide-pulsed T2 cells after the 3rd round of peptide stimulation on day 21 (Tanaka H et al., Br J Cancer 2001, 84(1): 94-9; Umano Y et al., Br J Cancer 2001, 84(8): 1052-7; Uchida N et al., Clin Cancer Res 2004, 10(24): 8577-86; Suda T et al., Cancer Sci 2006, 97(5): 411-9; Watanabe T et al., Cancer Sci 2005, 96(8): 498-506).

CTL Expansion Procedure

CTLs were expanded in culture using the method similar to the one described by Riddell et al. (Walter E A et al., N Engl J Med 1995, 333(16): 1038-44; Riddell S R et al., Nat Med 1996, 2(2): 216-23). A total of 5×10⁴ CTLs were suspended in 25 ml of AIM-V/5% AS medium with 2 kinds of human B-lymphoblastoid cell lines, inactivated by Mitomycin C, in the presence of 40 ng/ml of anti-CD3 monoclonal antibody (Pharmingen). One day after initiating the cultures, 120 IU/ml of IL-2 were added to the cultures. The cultures were fed with fresh AIM-V/5% AS medium containing 30 IU/ml of IL-2 on days 5, 8 and 11 (Tanaka H et al., Br J Cancer 2001, 84(1): 94-9; Umano Y et al., Br J Cancer 2001, 84(8): 1052-7; Uchida N et al., Clin Cancer Res 2004, 10(24): 8577-86; Suda T et al., Cancer Sci 2006, 97(5): 411-9; Watanabe T et al., Cancer Sci 2005, 96(8): 498-506).

Establishment of CTL Clones

The dilutions were made to have 0.3, 1, and 3 CTLs/well in 96 round-bottomed micro titer plate (Nalge Nunc International). CTLs were cultured with 1×10⁴ cells/well of 2 kinds of human B-lymphoblastoid cell lines, 30 ng/ml of anti-CD3 antibody, and 125 U/ml of IL-2 in a total of 150 micro-l/well of AIM-V Medium containing 5% AS. 50 micro-l/well of IL-2 were added to the medium 10 days later so to reach a final concentration of 125 U/ml IL-2. CTL activity was tested on the 14th day, and CTL clones were expanded using the same method as described above (Uchida N et al., Clin Cancer Res 2004, 10(24): 8577-86; Suda T et al., Cancer Sci 2006, 97(5): 411-9; Watanabe T et al., Cancer Sci 2005, 96(8): 498-506).

Specific CTL Activity

To examine specific CTL activity, interferon (IFN)-gamma enzyme-linked immunospot (ELISPOT) assay and IFN-gamma enzyme-linked immunosorbent assay (ELISA) were performed. Specifically, peptide-pulsed T2 (1×10⁴/well) was prepared as stimulator cells. Cultured cells in 48 wells were used as responder cells. IFN-gamma ELISPOT assay and IFN-gamma ELISA assay were performed under manufacture procedure.

Establishment of the Cells Forcibly Expressing Either or Both of the Target Gene and HLA-A02

The cDNA encoding an open reading frame of target genes or HLA-A*0201 was amplified by PCR. The PCR-amplified product was cloned into a vector. The plasmids were transfected into COST, which is the target genes and HLA-A*0201-null cell line, using lipofectamine 2000 (Invitrogen) according to the manufacturer's recommended procedures. After 2 days from transfection, the transfected cells were harvested with versene (Invitrogen) and used as the target cells (5×10⁴ cells/well) for CTL activity assay.

Results

Prediction of HLA-A02 Binding Peptides Derived from TTLL4

Table 2a and 2b show the HLA-A02 binding 9 mer and 10 mer peptides of TTLL4 in the order of high binding affinity. A total of 41 peptides with potential HLA-A02 binding ability were selected and examined to determine the epitope peptides.

TABLE 2a
HLA-A02 binding 9mer peptides derived from TTLL4

| Start Position | amino acid sequence | Kd (nM) | SEQ ID NO |
|---|---|---|---|
| 222 | FMWPNSTPV | 2 | 38 |
| 805 | FMHLTNYSV | 6 | 39 |
| 610 | KMSTVTPNI | 7 | 40 |
| 1163 | SLSTQTLPV | 10 | 41 |
| 575 | LIYSLFPNV | 15 | 42 |
| 1189 | SISDSLLAV | 16 | 43 |
| 66 | GLGPGLLGV | 32 | 44 |
| 864 | TIISSEPYV | 37 | 45 |
| 899 | NLKPWVLEV | 48 | 46 |
| 147 | SLPQKSLPV | 49 | 47 |
| 578 | SLFPNVPPT | 51 | 48 |
| 697 | FILPQDAKL | 52 | 49 |
| 1088 | SLPTSLLTI | 70 | 50 |
| 988 | KIPDQDFYA | 79 | 51 |
| 423 | LLASHASGL | 163 | 52 |
| 852 | SIWEKTKDV | 200 | 53 |
| 128 | YQQLESFCL | 265 | 54 |
| 107 | SLPDLFNST | 278 | 55 |
| 605 | KLLRWKMST | 325 | 56 |
| 356 | CQLEQSSFL | 1503 | 57 |

TABLE 2b
HLA-A02 binding 10mer peptides derived from TTLL4

| Start Position | amino acid sequence | Kd (nM) | SEQ ID NO |
|---|---|---|---|
| 363 | FLNPSFQWNV | 3 | 58 |
| 574 | ALIYSLFPNV | 4 | 59 |
| 895 | MLDENLKPWV | 10 | 60 |
| 605 | KLLRWKMSTV | 16 | 61 |
| 578 | SLFPNVPPTI | 19 | 62 |
| 756 | YLISGSKFDL | 37 | 63 |
| 550 | AMISRSCMEI | 39 | 64 |
| 610 | KMSTVTPNIV | 42 | 65 |
| 107 | SLPDLFNSTL | 46 | 66 |
| 933 | NLAGFVLPNA | 56 | 67 |
| 1163 | SLSTQTLPVI | 59 | 68 |
| 871 | YVTSLLKMYV | 94 | 69 |
| 863 | KTIISSEPYV | 118 | 70 |
| 852 | SIWEKIKDVV | 150 | 71 |
| 62 | TLSAGLGPGL | 188 | 72 |
| 804 | KFMHLTNYSV | 192 | 73 |
| 70 | GLLGVPPQPA | 230 | 74 |
| 1092 | SLLTISKDDV | 292 | 75 |
| 1113 | KLGKQSSCEV | 324 | 76 |
| 778 | RIYLFSDGLV | 358 | 77 |
| 86 | TLCSSGTTAV | 421 | 78 |

Start position indicates the number of amino acid residue from the N-terminus of TTLL4.

Dissociation constant [Kd (nM)] is derived from “NetMHC3.0”.

CTL Induction with the Predicted Peptides from TTLL4 Restricted with HLA-A*0201

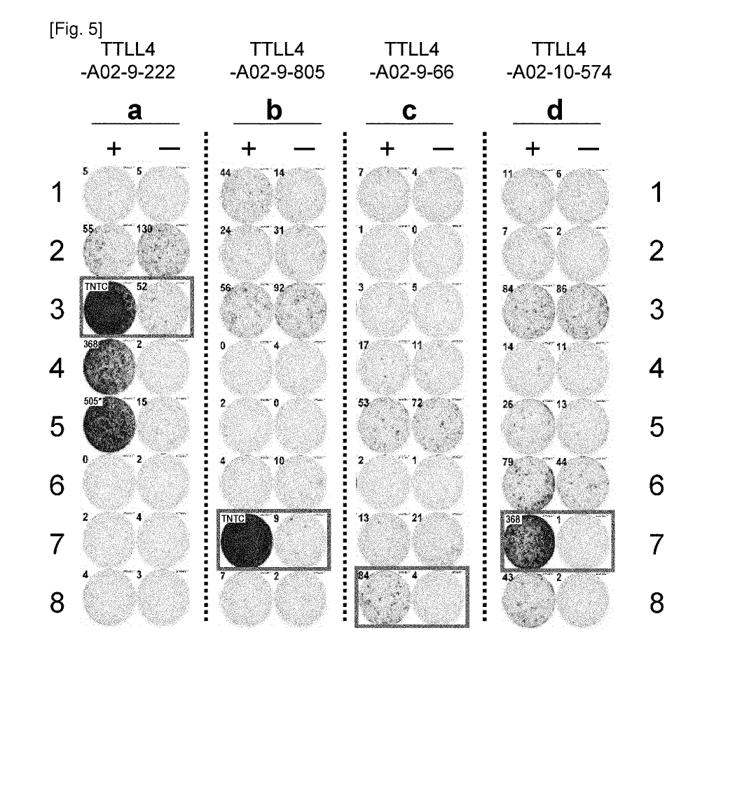

CTLs for those peptides derived from TTLL4 were generated according to the protocols as described in “Materials and Methods”. Peptide specific CTL activity was determined by IFN-gamma ELISPOT assay (FIG. 5a-d). Well number #3 with TTLL4-A02-9-222 (SEQ ID NO:38) (a), #7 with TTLL4-A02-9-805 (SEQ ID NO:39) (b), #8 with TTLL4-A02-9-66 (SEQ ID NO:44) (c) and #7 with TTLL4-A02-10-574 (SEQ ID NO:59) (d) demonstrated potent IFN-gamma production as compared to the control wells. On the other hand, no specific CTL activity was determined by stimulation with other peptides shown in Table 2a and 2b, despite those peptides had possible binding activity with HLA-A*0201. The results indicate that 4 peptides derived from TTLL4 were screened as the peptides that could induce potent CTLs.

Establishment of CTL Lines and Clones Against TTLL4 Derived Peptide

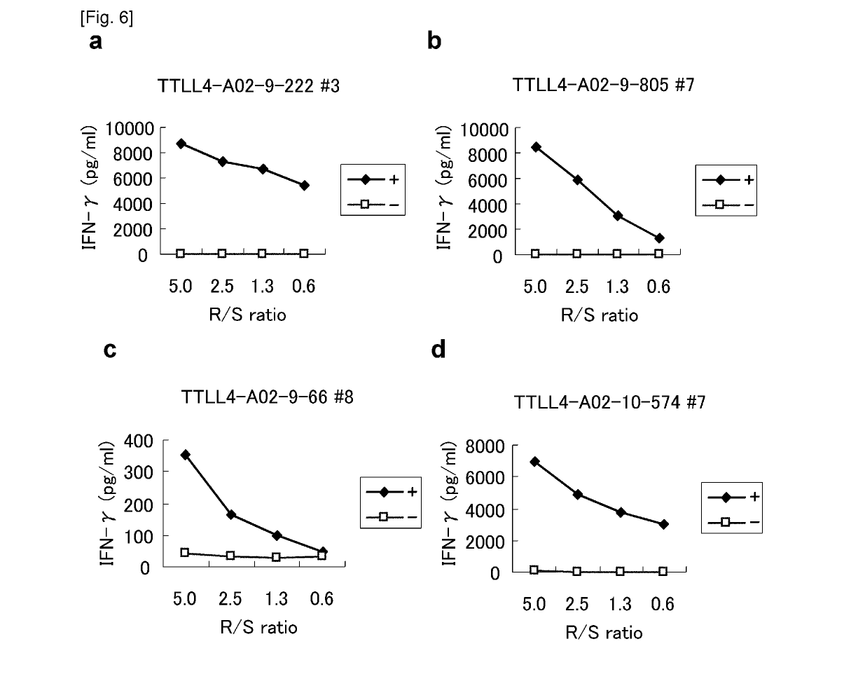

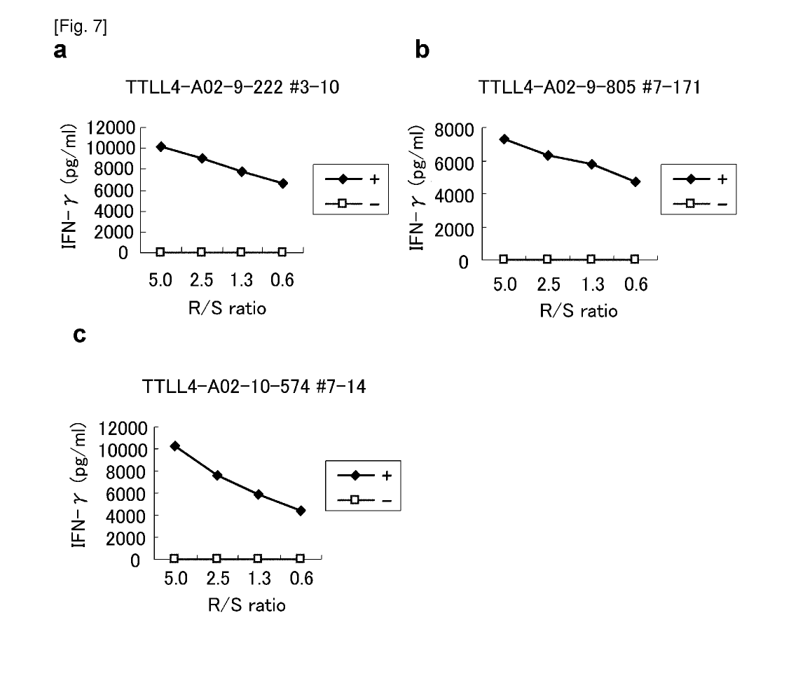

The cells that showed peptide specific CTL activity detected by IFN-gamma ELISPOT assay in the well number #3 with TTLL4-A02-9-222 (SEQ ID NO:38) (a), #7 with TTLL4-A02-9-805 (SEQ ID NO:39) (b), #8 with TTLL4-A02-9-66 (SEQ ID NO:44) (c) and #7 with TTLL4-A02-10-574 (SEQ ID NO:59) (d) were expanded and CTL lines were established as described in the Materials and Methods section above. CTL activity of these CTL lines was determined by IFN-gamma ELISA assay (FIG. 6a-d). The CTL lines demonstrated potent IFN-gamma production against the target cells pulsed with the corresponding peptide as compared to target cells without peptide pulse. Furthermore, the CTL clones were established by limiting dilution from the CTL lines as described in “Materials and Methods”, and IFN-gamma production from the CTL clones against target cells pulsed peptide was determined by IFN-gamma ELISA assay. Potent IFN-gamma productions were determined from the CTL clones stimulated with TTLL4-A02-9-222 (SEQ ID NO:38) (a), TTLL4-A02-9-805 (SEQ ID NO:39) (b) and TTLL4-A02-10-574 (SEQ ID NO:59) (c) (FIG. 7a-c).

Specific CTL Activity Against Target Cells Expressing TTLL4 and HLA-A*0201

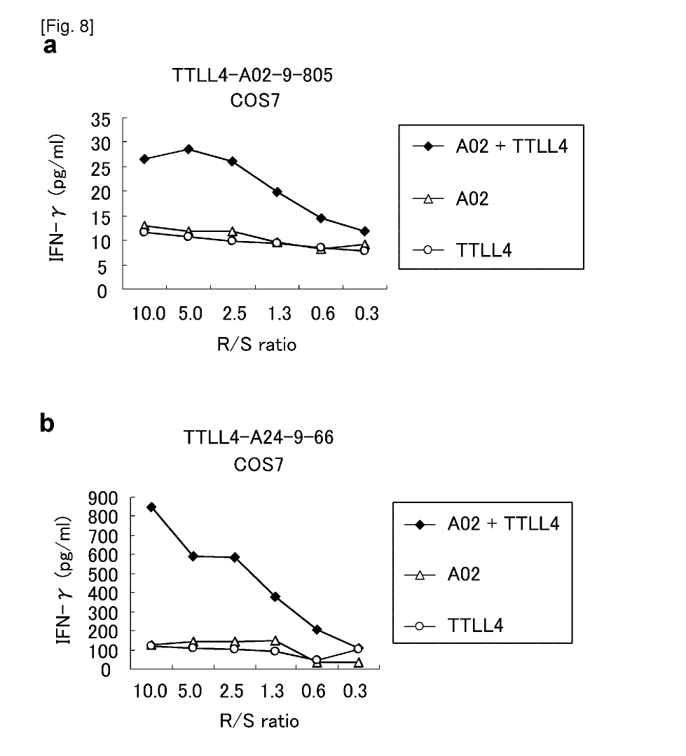

The established CTL lines and clones raised against each peptide were examined for the ability to recognize target cells that express TTLL4 and HLA-A*0201 molecule. Specific CTL activity against COS7 cells which transfected with both the full length of TTLL4 and HLA-A*0201 gene (a specific model for the responder cells that express TTLL4 and HLA-A*0201 gene) was tested by using the CTL lines and clones raised by corresponding peptide as the stimulator cells. COS7 cells transfected with either full length of TTLL4 or HLA-A*0201 were prepared as the controls. In FIG. 8, the CTL clone stimulated with TTLL4-A02-9-805 (SEQ ID NO: 39) (a) and the CTL line stimulated with TTLL4-A02-9-66 (SEQ ID NO: 44) (b) showed potent CTL activity against COS7 cells expressing both TTLL4 and HLA-A*0201. On the other hand, no significant specific CTL activity was detected against the controls. Thus, these data clearly demonstrated that peptides of TTLL4-A02-9-805 (SEQ ID NO: 39) (a) and TTLL4-A02-9-66 (SEQ ID NO: 44) (b) were endogenously processed and presented on the target cells with HLA-A*0201 molecule and were recognized by the CTLs. These results indicated that these peptides derived from TTLL4 may be suitable as a cancer vaccine for the treatment of patients with TTLL4 expressing tumors.

Homology Analysis of Antigen Peptides

The CTLs stimulated with TTLL4-A02-9-222 (SEQ ID NO:38), TTLL4-A02-9-805 (SEQ ID NO:39), TTLL4-A02-9-66 (SEQ ID NO:44) and TTLL4-A02-10-574 (SEQ ID NO:59) showed significant and specific CTL activity. This result may be due to the fact that the sequence of TTLL4-A02-9-222 (SEQ ID NO:38), TTLL4-A02-9-805 (SEQ ID NO:39), TTLL4-A02-9-66 (SEQ ID NO:44) and TTLL4-A02-10-574 (SEQ ID NO:59) are homologous to peptide derived from other molecules that are known to sensitize the human immune system. To exclude this possibility, homology analysis were performed for this peptide sequence using as queries the BLAST algorithm (http://www.ncbi.nlm.nih.gov/blast/blast.cgi) which revealed no sequence with significant homology. The results of homology analysis indicate that the sequence of TTLL4-A02-9-222 (SEQ ID NO:38), TTLL4-A02-9-66 (SEQ ID NO:44) and TTLL4-A02-10-574 (SEQ ID NO:59) are unique and thus, there is little possibility, to our best knowledge, that this molecules raise unintended immunologic response to some unrelated molecule. Although the sequence of TTLL4-A02-9-805 (SEQ ID NO: 39) is homologous to TTLL5, the expression profile of TTLL5 in our microarray data indicates that the TTLL5 expression is low in normal tissues and the peptide is applicable to cancer therapy.

In conclusion, novel HLA-A*0201 epitope peptides derived from TTLL4 were identified and demonstrated to be suitable for cancer immunotherapy.

### INDUSTRIAL APPLICABILITY

The present invention provides new TAAs, particularly those derived from TTLL4 that may induce potent and specific anti-tumor immune responses and have applicability to a wide variety of cancer types. Such TAAs can find utility as peptide vaccines against diseases associated with TTLL4, e.g., cancer, examples of which include, but are not limited to, bladder cancer, cholangiocellular carcinoma, CML, colon and rectum cancer, esophageal cancer, liver cancer, lymphoma, pancreatic cancer, prostate cancer, renal carcinoma, SCLC, NSCLC, soft tissue tumor and osteosarcoma.

While the present invention is herein described in detail and with reference to specific embodiments thereof, it is to be understood that the foregoing description is exemplary and explanatory in nature and is intended to illustrate the present invention and its preferred embodiments. Through routine experimentation, one skilled in the art will readily recognize that various changes and modifications can be made therein without departing from the spirit and scope of the present invention, the metes and bounds of which are defined by the appended claims.

1. (canceled)

2. An isolated peptide selected from the group consisting of:
(a) an isolated peptide, wherein the peptide comprises an amino acid sequence selected from the group consisting of SEQ ID NOs: 1, 6, 11, 12, 16, 20, 21, 22, 28, 29, 32, 37, 38, 39, 44 and 59; and
(b) an isolated peptide, wherein the peptide comprises an amino acid sequence in which 1, 2, or several amino acid(s) are substituted, deleted, inserted, and/or added in the amino acid sequence selected from the group consisting of SEQ ID NOs: 1, 6, 11, 12, 16, 20, 21, 22, 28, 29, 32, 37, 38, 39, 44 and 59, and wherein the peptide has cytotoxic T lymphocyte (CTL) inducibility.

3-5. (canceled)

6. The peptide of claim 2, wherein the peptide has one or both of the following characteristics:
(a) the second amino acid from the N-terminus of the amino acid sequence selected from the group consisting of SEQ ID NOs: 1, 6, 11, 12, 16, 20, 21, 22, 28, 29, 32, and 37 is selected from the group consisting of phenylalanine, tyrosine, methionine and tryptophan, and
(b) the C-terminal amino acid of the amino acid sequence selected from the group consisting of SEQ ID NOs: 1, 6, 11, 12, 16, 20, 21, 22, 28, 29, 32 and 37 is selected from the group consisting of phenylalanine, leucine, isoleucine, tryptophan and methionine.

7. The peptide of claim 2, wherein the peptide has one or both of the following characteristics:
(a) the second amino acid from the N-terminus of the amino acid sequence selected from the group consisting of SEQ ID NOs: 38, 39, 44 and 59 is selected from the group consisting of leucine and methionine; and (b) the C-terminal amino acid of the amino acid sequence selected from the group consisting of SEQ ID NOs: 38, 39, 44 and 59 is selected from the group consisting of valine and leucine.

8. The isolated peptide of claim 2, wherein said peptide is nonapeptide or decapeptide.

9. An isolated polynucleotide encoding the isolated peptide of claim 2.

10. A composition for inducing a CTL, wherein the composition comprises one or more of the peptide(s) of claim 2, or one or more of the polynucleotide(s) encoding the peptide.

11. A pharmaceutical composition for the treatment and/or prophylaxis of cancer, and/or the prevention of a postoperative recurrence thereof, wherein the composition comprises one or more of the peptide(s) of claim 2, or one or more of the polynucleotide(s) encoding the peptide.

12. The pharmaceutical composition of claim 11, wherein said composition is formulated for the administration to a subject whose HLA antigen is HLA-A24 or HLA-A2.

13. A method for inducing an antigen-presenting cell (APC) with CTL inducibility, said method comprising a step selected from the group consisting of:
(a) contacting an APC with the peptide of claim 2 in vitro, ex vivo or in vivo, and
(b) introducing a polynucleotide encoding the peptide of claim 2 into an APC.

14. A method for inducing a CTL, said method comprising a step selected from the group consisting of:
(a) co-culturing CD8 positive T cells with APCs that present on the surface a complex of an HLA antigen and the peptide of claim 2,
(b) co-culturing CD8 positive T cells with exosomes that present on the surface a complex of an HLA antigen and the peptide of claim 2, and
(c) introducing a gene that comprises a polynucleotide encoding a T cell receptor (TCR) subunit polypeptide bound to the peptide of claim 2 into a T cell.

15. An isolated APC that presents on its surface a complex of an HLA antigen and the peptide of claim 2.

16. An isolated APC, which is induced by the method of claim 13.

17. An isolated CTL that targets the peptide of claim 2.

18. An isolated CTL, which is induced by the method of claim 14.

19. A method of inducing immune response against cancer in a subject in need thereof, said method comprising the step of administering to the subject a composition comprising the peptide of claim 2, an immunologically active fragment thereof, or a polynucleotide encoding the peptide or the fragment.

20. An antibody or immunologically active fragment thereof against the peptides of claim 2.

21. A vector comprising a nucleotide sequence encoding the peptides of claim 2.

22. A host cell transformed or transfected with an expression vector according to claim 21.

23. A diagnostic kit comprising the peptides of claim 2, a polynucleotide encoding the peptide or an antibody against the peptide.

24. (canceled)


SEQUENCE LISTING (84 sequences)

SEQ ID NO: 1 - protein, 9 aa, Artificial Sequence
  An artificially synthesized peptide sequence
RYLHKPYLI

SEQ ID NO: 2 - protein, 9 aa, Artificial Sequence
  An artificially synthesized peptide sequence
LFPNVPPTI

SEQ ID NO: 3 - protein, 9 aa, Artificial Sequence
  An artificially synthesized peptide sequence
FYASVLDVL

SEQ ID NO: 4 - protein, 9 aa, Artificial Sequence
  An artificially synthesized peptide sequence
VYVTSYDPL

SEQ ID NO: 5 - protein, 9 aa, Artificial Sequence
  An artificially synthesized peptide sequence
PYLISGSKF

SEQ ID NO: 6 - protein, 9 aa, Artificial Sequence
  An artificially synthesized peptide sequence
AYFFCPSTL

SEQ ID NO: 7 - protein, 9 aa, Artificial Sequence
  An artificially synthesized peptide sequence
RFGKKEFSF

SEQ ID NO: 8 - protein, 9 aa, Artificial Sequence
  An artificially synthesized peptide sequence
EFSFFPQSF

SEQ ID NO: 9 - protein, 9 aa, Artificial Sequence
  An artificially synthesized peptide sequence
IYLFSDGLV

SEQ ID NO: 10 - protein, 9 aa, Artificial Sequence
  An artificially synthesized peptide sequence
WYNRNNLAM

SEQ ID NO: 11 - protein, 9 aa, Artificial Sequence
  An artificially synthesized peptide sequence
KYSPSMKSL

SEQ ID NO: 12 - protein, 9 aa, Artificial Sequence
  An artificially synthesized peptide sequence
SFFPQSFIL

SEQ ID NO: 13 - protein, 9 aa, Artificial Sequence
  An artificially synthesized peptide sequence
VWPQAHQQV

SEQ ID NO: 14 - protein, 9 aa, Artificial Sequence
  An artificially synthesized peptide sequence
VWSLPTSLL

SEQ ID NO: 15 - protein, 9 aa, Artificial Sequence
  An artificially synthesized peptide sequence
TFQSISDSL

SEQ ID NO: 16 - protein, 9 aa, Artificial Sequence
  An artificially synthesized peptide sequence
CYLHSLPDL

SEQ ID NO: 17 - protein, 9 aa, Artificial Sequence
  An artificially synthesized peptide sequence
SFLNPSFQW

SEQ ID NO: 18 - protein, 9 aa, Artificial Sequence
  An artificially synthesized peptide sequence
RFFEQPRYF

SEQ ID NO: 19 - protein, 9 aa, Artificial Sequence
  An artificially synthesized peptide sequence
SYDPLRIYL

SEQ ID NO: 20 - protein, 10 aa, Artificial Sequence
  An artificially synthesized peptide sequence
CYLHSLPDLF

SEQ ID NO: 21 - protein, 10 aa, Artificial Sequence
  An artificially synthesized peptide sequence
SYDPLRIYLF

SEQ ID NO: 22 - protein, 10 aa, Artificial Sequence
  An artificially synthesized peptide sequence
PYSCHELFGF

SEQ ID NO: 23 - protein, 10 aa, Artificial Sequence
  An artificially synthesized peptide sequence
PYQQLESFCL

SEQ ID NO: 24 - protein, 10 aa, Artificial Sequence
  An artificially synthesized peptide sequence
RFGKKEFSFF

SEQ ID NO: 25 - protein, 10 aa, Artificial Sequence
  An artificially synthesized peptide sequence
RYFNILTTQW

SEQ ID NO: 26 - protein, 10 aa, Artificial Sequence
  An artificially synthesized peptide sequence
MWPNSTPVPL

SEQ ID NO: 27 - protein, 10 aa, Artificial Sequence
  An artificially synthesized peptide sequence
SYRQKPYQQL

SEQ ID NO: 28 - protein, 10 aa, Artificial Sequence
  An artificially synthesized peptide sequence
TFQSISDSLL

SEQ ID NO: 29 - protein, 10 aa, Artificial Sequence
  An artificially synthesized peptide sequence
QFERIFPSHI

SEQ ID NO: 30 - protein, 10 aa, Artificial Sequence
  An artificially synthesized peptide sequence
EFSFFPQSFI

SEQ ID NO: 31 - protein, 10 aa, Artificial Sequence
  An artificially synthesized peptide sequence
KFMHLTNYSV

SEQ ID NO: 32 - protein, 10 aa, Artificial Sequence
  An artificially synthesized peptide sequence
FYASVLDVLT

SEQ ID NO: 33 - protein, 10 aa, Artificial Sequence
  An artificially synthesized peptide sequence
DFYASVLDVL

SEQ ID NO: 34 - protein, 10 aa, Artificial Sequence
  An artificially synthesized peptide sequence
AFSKSETSKL

SEQ ID NO: 35 - protein, 10 aa, Artificial Sequence
  An artificially synthesized peptide sequence
SFILPQDAKL

SEQ ID NO: 36 - protein, 10 aa, Artificial Sequence
  An artificially synthesized peptide sequence
SFQIGRKDRL

SEQ ID NO: 37 - protein, 10 aa, Artificial Sequence
  An artificially synthesized peptide sequence
GFDIMLDENL

SEQ ID NO: 38 - protein, 9 aa, Artificial Sequence
  An artificially synthesized peptide sequence
FMWPNSTPV

SEQ ID NO: 39 - protein, 9 aa, Artificial Sequence
  An artificially synthesized peptide sequence
FMHLTNYSV

SEQ ID NO: 40 - protein, 9 aa, Artificial Sequence
  An artificially synthesized peptide sequence
KMSTVTPNI

SEQ ID NO: 41 - protein, 9 aa, Artificial Sequence
  An artificially synthesized peptide sequence
SLSTQTLPV

SEQ ID NO: 42 - protein, 9 aa, Artificial Sequence
  An artificially synthesized peptide sequence
LIYSLFPNV

SEQ ID NO: 43 - protein, 9 aa, Artificial Sequence
  An artificially synthesized peptide sequence
SISDSLLAV

SEQ ID NO: 44 - protein, 9 aa, Artificial Sequence
  An artificially synthesized peptide sequence
GLGPGLLGV

SEQ ID NO: 45 - protein, 9 aa, Artificial Sequence
  An artificially synthesized peptide sequence
TIISSEPYV

SEQ ID NO: 46 - protein, 9 aa, Artificial Sequence
  An artificially synthesized peptide sequence
NLKPWVLEV

SEQ ID NO: 47 - protein, 9 aa, Artificial Sequence
  An artificially synthesized peptide sequence
SLPQKSLPV

SEQ ID NO: 48 - protein, 9 aa, Artificial Sequence
  An artificially synthesized peptide sequence
SLFPNVPPT

SEQ ID NO: 49 - protein, 9 aa, Artificial Sequence
  An artificially synthesized peptide sequence
FILPQDAKL

SEQ ID NO: 50 - protein, 9 aa, Artificial Sequence
  An artificially synthesized peptide sequence
SLPTSLLTI

SEQ ID NO: 51 - protein, 9 aa, Artificial Sequence
  An artificially synthesized peptide sequence
KIPDQDFYA

SEQ ID NO: 52 - protein, 9 aa, Artificial Sequence
  An artificially synthesized peptide sequence
LLASHASGL

SEQ ID NO: 53 - protein, 9 aa, Artificial Sequence
  An artificially synthesized peptide sequence
SIWEKIKDV

SEQ ID NO: 54 - protein, 9 aa, Artificial Sequence
  An artificially synthesized peptide sequence
YQQLESFCL

SEQ ID NO: 55 - protein, 9 aa, Artificial Sequence
  An artificially synthesized peptide sequence
SLPDLFNST

SEQ ID NO: 56 - protein, 9 aa, Artificial Sequence
  An artificially synthesized peptide sequence
KLLRWKMST

SEQ ID NO: 57 - protein, 9 aa, Artificial Sequence
  An artificially synthesized peptide sequence
CQLEQSSFL

SEQ ID NO: 58 - protein, 10 aa, Artificial Sequence
  An artificially synthesized peptide sequence
FLNPSFQWNV

SEQ ID NO: 59 - protein, 10 aa, Artificial Sequence
  An artificially synthesized peptide sequence
ALIYSLFPNV

SEQ ID NO: 60 - protein, 10 aa, Artificial Sequence
  An artificially synthesized peptide sequence
MLDENLKPWV

SEQ ID NO: 61 - protein, 10 aa, Artificial Sequence
  An artificially synthesized peptide sequence
KLLRWKMSTV

SEQ ID NO: 62 - protein, 10 aa, Artificial Sequence
  An artificially synthesized peptide sequence
SLFPNVPPTI

SEQ ID NO: 63 - protein, 10 aa, Artificial Sequence
  An artificially synthesized peptide sequence
YLISGSKFDL

SEQ ID NO: 64 - protein, 10 aa, Artificial Sequence
  An artificially synthesized peptide sequence
AMISRSCMEI

SEQ ID NO: 65 - protein, 10 aa, Artificial Sequence
  An artificially synthesized peptide sequence
KMSTVTPNIV

SEQ ID NO: 66 - protein, 10 aa, Artificial Sequence
  An artificially synthesized peptide sequence
SLPDLFNSTL

SEQ ID NO: 67 - protein, 10 aa, Artificial Sequence
  An artificially synthesized peptide sequence
NLAGFVLPNA

SEQ ID NO: 68 - protein, 10 aa, Artificial Sequence
  An artificially synthesized peptide sequence
SLSTQTLPVI

SEQ ID NO: 69 - protein, 10 aa, Artificial Sequence
  An artificially synthesized peptide sequence
YVTSLLKMYV

SEQ ID NO: 70 - protein, 10 aa, Artificial Sequence
  An artificially synthesized peptide sequence
KTIISSEPYV

SEQ ID NO: 71 - protein, 10 aa, Artificial Sequence
  An artificially synthesized peptide sequence
SIWEKIKDVV

SEQ ID NO: 72 - protein, 10 aa, Artificial Sequence
  An artificially synthesized peptide sequence
TLSAGLGPGL

SEQ ID NO: 73 - protein, 10 aa, Artificial Sequence
  An artificially synthesized peptide sequence
KFMHLTNYSV

SEQ ID NO: 74 - protein, 10 aa, Artificial Sequence
  An artificially synthesized peptide sequence
GLLGVPPQPA

SEQ ID NO: 75 - protein, 10 aa, Artificial Sequence
  An artificially synthesized peptide sequence
SLLTISKDDV

SEQ ID NO: 76 - protein, 10 aa, Artificial Sequence
  An artificially synthesized peptide sequence
KLGKQSSCEV

SEQ ID NO: 77 - protein, 10 aa, Artificial Sequence
  An artificially synthesized peptide sequence
RIYLFSDGLV

SEQ ID NO: 78 - protein, 10 aa, Artificial Sequence
  An artificially synthesized peptide sequence
TLCSSGTTAV

SEQ ID NO: 79 - DNA, 4997 bp, Homo sapiens
  CDS (371)..(3970)
agactctcggtctgtccgctgggggcgcgcgcggtgtgtggcaggcggcagcggcgctggcggccgagtgcgcttgtcacgcgtggcggtgcgtggttgctaggggcgcctgaggctgccgggtagcccagcaggccgagggaggaagtagcgtggagccggtgccgagccggggcgaagctggatcccctagatagactgtcttcaagctcactgatattttcctctgcttgatccattgtgctgttgagagcctctagtaaatttttcagactgacagacttcaaggatgcagctgctactaccggaggtgtgtggcaccttacctcagcaaggccatgagaccgtgtggccatgatgtgggcccctcatggcctcagcaggaacacagcactatagtattggcctccgccagaaaaacagcttcaagcagagtggtccctcaggcacagtacctgccacgccacctgagaaaccctcggagggcagagtctggcctcaggcccatcagcaagtgaagccaatctggaagctggaaaagaagcaagtggagacactgtcagcagggttgggcccaggcctcttgggcgtcccaccccagccagcatatttcttttgccccagcactttatgtagctctgggaccacggctgtcattgcaggccacagcagttcctgttacctacactctctcccggacttgttcaacagcaccctgctataccgccgctccagctataggcaaaaaccgtaccagcaactggagtctttctgcttgcgttcgagcccgtcagaaaaaagccctttttctctccctcaaaagagcctccctgtcagtctcactgccaacaaggccacttcttccatggtcttctccatggcccagcccatggcctcctcatccacagaaccatacctctgcttggcagcggctggggaaaacccttcagggaagagcctggcctctgccatctcagggaagatcccatctccactctcttcctcctataagcccatgctgaataataattccttcatgtggccaaatagcacgccagtgcctttattgcagaccacacagggcctgaagccagtatcgccacccaagatccagcctgtctcctggcatcattcagggggtactggagactgtgcaccgcagcctgttgaccataaggtgcccaaaagcattggcactgtcccagctgatgccagtgcccatatcgccttgtctaccgctagctcccacgacacatccaccaccagtgttgcctcttcctggtataaccggaataacttagccatgagggcagagccactttcctgtgctctggatgacagctctgattcccaggatccaactaaggagattcggttcactgaggccgtgaggaaattgaccgcaagaggctttgagaagatgccgaggcaaggctgccagcttgaacagtctagtttcctgaaccccagcttccagtggaatgtcctcaacaggagcaggcggtggaaacctcctgcggtaaatcagcagtttcctcaggaggatgctggatcggtcaggcgggtcctccctggtgcctcagataccttggggttggacaatacagtcttctgtaccaagcgtatcagcattcacctccttgcctcacatgccagtgggctcaatcacaaccctgcctgtgaatctgtaattgactcctcagcatttggagaaggcaaagctccaggtcccccttttcctcaaactcttggcatagccaacgtggccacccgcctctcttccatccagctgggccagtctgagaaggagagacctgaggaggccagggagctggactcatctgatagggatattagttcagctactgacctccagccagatcaggctgagactgaagatacagaagaagaactagtagatggtttggaagactgttgtagccgtgatgagaatgaagaggaggagggagactcagagtgctcctcattaagtgctgtctcccccagcgaatcggtggccatgatctctagaagctgtatggaaattctgaccaaacccctttccaatcatgagaaagttgtccgaccagccctcatctacagtctctttcccaacgttccccctaccatctattttggcactcgggatgagagagtggagaaacttccctgggaacagaggaagttgctccgatggaagatgagcacagtgacccccaacattgtcaagcagaccattggacggtcccacttcaaaatcagcaaaagaaacgatgactggctgggctgctggggtcaccacatgaagtctcctagtttccgatccattcgagagcatcagaagctaaaccatttcccaggctcattccagattgggaggaaggaccggctatggcggaacctgtcacgtatgcagagccgctttggcaagaaggagttcagtttcttcccccagtcctttatcctgccccaggacgccaagctcctgcgcaaagcgtgggagagcagcagccgccaaaagtggattgtgaagccaccagcatcagctcgaggcattggcatccaggttattcacaagtggagtcagctccccaagcgaaggcccctcctggtacagaggtatctacacaaaccctacctcatcagcggcagcaagtttgacctgcggatctatgtttatgtcacttcctacgatcctctgcggatttacctcttttcagatggactggtccgctttgccagttgcaagtattcgccttccatgaagagccttggcaataagttcatgcacctgaccaactacagtgtcaataaaaagaatgccgagtaccaggccaatgcagatgaaatggcttgccagggccacaaatgggcactgaaggctttgtggaactacctgagccagaagggagtcaatagcgacgccatctgggagaagataaaggatgttgttgtcaaaactatcatctcgtcagagccctatgtgaccagcctgctcaagatgtatgtgcgacggccctatagctgccatgaactctttggttttgacatcatgctagacgaaaacctcaagccctgggtcctggaagtcaacatttccccaagcctccactccagctctccactggatatcagcatcaaaggccagatgattcgtgaccttctgaatctggcaggttttgtcctgcccaatgcagaggatatcatttccagccccagcagctgcagcagctccaccaccagcctgcccacctcccctggggacaaatgtcgaatggctccagagcatgtcactgcacagaagatgaagaaagcctattatctgacccagaaaattcctgatcaggacttctatgcatctgtgctggatgtcctgacaccagatgatgttcggattctggttgagatggaagatgagttttctcgccgtggtcagtttgaacgaatttttccttctcatatctcctctcgctatctccgcttttttgagcagccacgatatttcaacattctcaccacccaatgggaacagaaataccatggcaacaagcttaaaggagtagatctgctccggagttggtgctacaaagggttccacatgggagttgtctctgattctgctccagtgtggtctctcccgacatcacttctgactatctcaaaggatgacgtgatactcaatgccttcagcaaatcagagactagcaagctgggaaaacaaagctcctgtgaggttagcctactactctctgaagacgggaccacgcccaaatccaagaagactcaagctggcctttccccttatccccagaaacccagttcctcaaaggacagtgaggacaccagcaaagagcccagcctttctacccagacgttacctgtgatcaagtgctctgggcagacttcaagactttctgcttcctccactttccagtcaatcagtgactccctcctggctgtgagcccataactggcctctctccaaaagcctctgcccaggagcatgggcatcagctacctcacgggaaccagcctgctgttcagaccagtctgaccccctacccctttcaccctgtccctcctcagagtattttttgaagtggttgcattatagagatgggtatttgtagggccggagggatggtagtgatggggagaaggtgaggaagggtcaccctctgtcacctgtctgcctggctggcacctcatatctcagcagagaagccagtggtggccacgcagccttataaagcaggttttggtttctaccttaagtgagccatgtgtggtttgtctgggggccctggtgtggttgctgagttgtagctcaagaggagaaaacatacagaacatatttggaccggaaatcctttgttctgaatttgagggggtcttctgaggtccttacttccttaggtctttcctcacccctctcccaccgctgtcctgaggagaaacccttgaacttcctcagtagacaggcggagaggccacaacatgccgaacccatttcctgtcatcctagtcttgggtcttcaccgcctccttccaaatacccaccctgccagcagccctaggtcttcctgttctgaccccccatcactgctcgttcagccttctagatgtctctctcgtggacatctgttctttagctgttggctttctctgaggtgtgagagggtctatgaactttgtgaatttcccatggccccagtgaaggagcccagataatcccagtagctgttacctgtctccatgtatcaaaggacacagtccagggggagggtggaaggagatgtggtttctctatagtgcaacaaacatggtttctcaatgttctgctgtgcagcaagcagggtctggcggcttggtaggtgggtttcaggagcagtcactattgtaggatgggcttccaatcaaacctcagactaaactcttgtactgaactgattctacctccctcctctagactcagtaaacagtgactattcaataaa

SEQ ID NO: 80 - protein, 1199 aa, Homo sapiens
MASAGTQHYSIGLRQKNSFKQSGPSGTVPATPPEKPSEGRVWPQAHQQVKPIWKLEKKQVETLSAGLGPGLLGVPPQPAYFFCPSTLCSSGTTAVIAGHSSSCYLHSLPDLFNSTLLYRRSSYRQKPYQQLESFCLRSSPSEKSPFSLPQKSLPVSLTANKATSSMVFSMAQPMASSSTEPYLCLAAAGENPSGKSLASAISGKIPSPLSSSYKPMLNNNSFMWPNSTPVPLLQTTQGLKPVSPPKIQPVSWHHSGGTGDCAPQPVDHKVPKSIGTVPADASAHIALSTASSHDTSTTSVASSWYNRNNLAMRAEPLSCALDDSSDSQDPTKEIRFTEAVRKLTARGFEKMPRQGCQLEQSSFLNPSFQWNVLNRSRRWKPPAVNQQFPQEDAGSVRRVLPGASDTLGLDNTVFCTKRISIHLLASHASGLNHNPACESVIDSSAFGEGKAPGPPFPQTLGIANVATRLSSIQLGQSEKERPEEARELDSSDRDISSATDLQPDQAETEDTEEELVDGLEDCCSRDENEEEEGDSECSSLSAVSPSESVAMISRSCMEILTKPLSNHEKVVRPALIYSLFPNVPPTIYFGTRDERVEKLPWEQRKLLRWKMSTVTPNIVKQTIGRSHFKISKRNDDWLGCWGHHMKSPSFRSIREHQKLNHFPGSFQIGRKDRLWRNLSRMQSRFGKKEFSFFPQSFILPQDAKLLRKAWESSSRQKWIVKPPASARGIGIQVIHKWSQLPKRRPLLVQRYLHKPYLISGSKFDLRIYVYVTSYDPLRIYLFSDGLVRFASCKYSPSMKSLGNKFMHLTNYSVNKKNAEYQANADEMACQGHKWALKALWNYLSQKGVNSDAIWEKIKDVVVKTIISSEPYVTSLLKMYVRRPYSCHELFGFDIMLDENLKPWVLEVNISPSLHSSSPLDISIKGQMIRDLLNLAGFVLPNAEDIISSPSSCSSSTTSLPTSPGDKCRMAPEHVTAQKMKKAYYLTQKIPDQDFYASVLDVLTPDDVRILVEMEDEFSRRGQFERIFPSHISSRYLRFFEQPRYFNILTTQWEQKYHGNKLKGVDLLRSWCYKGFHMGVVSDSAPVWSLPTSLLTISKDDVILNAFSKSETSKLGKQSSCEVSLLLSEDGTTPKSKKTQAGLSPYPQKPSSSKDSEDTSKEPSLSTQTLPVIKCSGQTSRLSASSTFQSISDSLLAVSP

SEQ ID NO: 81 - DNA, 22 bp, Artificial Sequence
  Artificial sequence
gtctaccaggcattcgcttcat

SEQ ID NO: 82 - DNA, 24 bp, Artificial Sequence
  Artificial sequence
tcagctggaccacagccgcagcgt

SEQ ID NO: 83 - DNA, 21 bp, Artificial Sequence
  Artificial sequence
tcagaaatcctttctcttgac

SEQ ID NO: 84 - DNA, 24 bp, Artificial Sequence
  Artificial sequence
ctagcctctggaatcctttctctt